(19) United States
(12) Patent Application Publication
(10) Pub. No.: US 2021/0128566 A1
(43) Pub. Date: May 6, 2021

(54) NOVEL FUNCTIONALIZED PURINE-2,6-DIONES AND THEIR USE IN MEDICINE

(71) Applicant: THOMAS HELLEDAYS STIFTELSE FÖR MEDICINSK FORSKNING, Stockholm (SE)

(72) Inventors: Brent PAGE, Solna (SE); Nicholas VALERIE, Solna (SE); Thomas HELLEDAY, Solna (SE); Olov WALLNER, Solna (SE); Martin SCOBIE, Solna (SE); Sabin LLONA-MINGUEZ, Solna (SE)

(21) Appl. No.: 16/633,260

(22) PCT Filed: Jul. 27, 2018
(86) PCT No.: PCT/EP2018/070522

§ 371 Date: Jan. 23, 2020

(30) Foreign Application Priority Data
Jul. 27, 2017 (GB) 1712110.4

(51) Int. Cl. A61K 31/522; A61K 45/06; A61P 35/00; C07D 473/08; C07D 519/00; C07D 471/04; A61K 31/5377; A61K 31/55; A61K 31/551

(52) U.S. Cl. CPC A61K 31/522; A61K 45/06; A61P 35/00; C07D 473/08; A61K 31/551; C07D 471/04; A61K 31/5377; A61K 31/55; C07D 519/00

(57) ABSTRACT

There is provided compounds of formula (I), or pharmaceutically-acceptable salts thereof, wherein R¹ to R⁴ and X¹ to X⁵ have meanings provided in the description, which compounds are useful in the treatment of cancers.

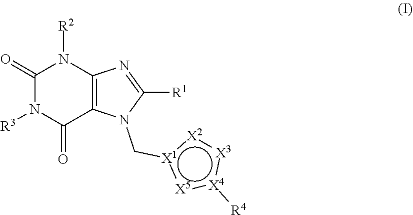

### FIELD OF THE INVENTION

The present invention relates to novel compounds, compositions comprising such compounds, and the use of such compounds and compositions in the treatment of cancers. In particular, the invention relates to the use of such compounds and compositions in methods for the treatment of cancers, such as breast cancer, through the inhibition of NUDT5.

### BACKGROUND OF THE INVENTION

The listing or discussion of an apparently prior-published document in this specification should not necessarily be taken as an acknowledgement that the document is part of the state of the art or is common general knowledge.

Although the increased understanding of the role of oncogenes, and the development of new anticancer treatments and diagnosis, have improved the life expectancy of cancer patients, there is still a high medical need to find more effective and less toxic treatments for cancers.

The tumour microenvironment is a critical hub for the growth and proliferation of cancer cells. The hormones and growth factors that may be present within this region are involved in the initiation of several intracellular processes that are involved in gene expression and thus dictate the fate of the cancer cell.

In order for activated transcription factors to bind DNA and begin expressing their target genes, the DNA must be unwound to facilitate access to critical transcription sites. This process depends on the activity of poly-adenosine diphosphate ribose (poly-ADPR)-related enzymes, such as poly(ADP-ribose) polymerase (PARP), poly(ADP-ribose) glycohydrolase (PARG) and NUDIX-type enzyme 5 (NUDT5), where “NUDIX” indicates the type of substrate for the enzyme and represents nucleoside diphosphate linked to X.

Activation of PARP causes the formation of poly-ADPR, which helps to recruit a host of factors that are important for transcription. Once these factors are recruited, PARG breaks down poly-ADPR into shorter ADPR oligomers and individual ADPR units. NUDT5 (also known as NUDIX5) was recently identified as a key factor for nuclear ATP production (Wright, R. H. G. et al. Science 2016, 352, 1221-1225.). Previously, NUDT5 was thought to be involved in 8-oxo-dGDP metabolism and in the hydrolysis of ADPR to form AMP and ribose-5-phosphate (Ito, R. et al. J Biochem 2011, 149 (6), 731-738). While it has been known that isolated nuclei could produce ATP from poly(ADPR) for a number of years (Tanuma, S. I. Biochem. Biophys. Res. Commun. 1989, 163 (2) 1047-1055), the precise mechanism and key enzymes have been elusive. Identification of NUDT5 as a key player in the production of nuclear ATP which is required for hormone-dependent gene expression in cancer cells (Wright, R. H. G. et al. Science 2016, 352, 1221-1225). The disruption of ATP production and therefore gene expression may result in growth arrest in hormone-dependent cancer cells.

EP 2 930 238 A1 refers to the use of NUDIX5 inhibitors in the treatment or prevention of cancers; however, no specific compounds are disclosed.

Accordingly, the inhibition of NUDT5 is an appealing target for chemotherapy, particularly in hormone-dependent cancer cells.

### DETAILED DESCRIPTION OF THE INVENTION

It has now surprisingly been found that certain purines bearing an alkyl-linked heterocyclic substituent in the 7-position are able to inhibit the action of NUDT5, and thus have properties useful for the treatment of cancers.

### Compounds of the Invention

In a first aspect of the invention, there is provided a compound of formula I

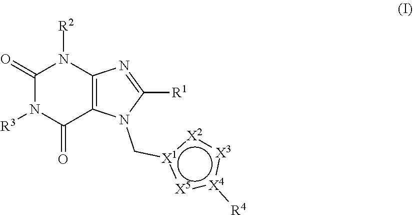

or a pharmaceutically-acceptable salt thereof, wherein:
any one to three of X¹ to X⁵ represents a heteroatom selected from N, O and S, with the provisos that
only one of X¹ to X⁵ may represent O or S,
if representing a heteroatom, X¹ and X⁴ may only represent N, and
X¹ and X⁴ may not both represent N,
   with the remainder of X¹ and X⁴ representing C, and the remainder of X², X³ and X⁵ representing CR⁵;
   R¹ represents
   (i) heteroaryl optionally substituted by one or more groups selected from E¹, or heterocyclyl optionally substituted by one or more groups independently selected from E², such as wherein each such heteroaryl or heterocyclyl group is attached via a constituent heteroatom,
   (ii) —NR^{a1}R^{a2}, —OR^{a3}, —S(O)ₚR^{a4} or —S(O)_{q}NR^{a5},
   (iii) C₁₋₁₀ alkyl, C₂₋₁₀ alkenyl or C₂₋₁₀ alkynyl, wherein each such alkyl, alkenyl or alkynyl group is optionally substituted by one or more groups independently selected from E³,
   (iv) aryl optionally substituted by one or more groups independently selected from E⁴, or
   (v) halo;
   R² and R³ each independently represent H, C₁₋₄ alkyl, C₂₋₄ alkenyl or C₂₋₄ alkynyl, wherein each such alkyl, alkenyl or alkynyl group is optionally substituted by one or more groups independently selected from G^{a1};
   R⁴ represents aryl optionally substituted by one or more groups independently selected from E⁵, or heteroaryl optionally substituted by one or more groups independently selected from E⁶;
   R⁵ represents H, C₁₋₄ alkyl, C₂₋₄ alkenyl or C₂₋₄ alkynyl, wherein each such alkyl, alkenyl or alkynyl group is optionally substituted by one or more groups independently selected from G^{a2}, aryl optionally substituted by one or more groups independently selected from G^{a3}, heterocycyl optionally substituted by one or more groups independently selected from G^{a4}, or heteroaryl optionally substituted by one or more groups independently selected from G^{a5};
   R^{a1} to R^{a5} each independently represent C₁₋₁₀ alkyl, C₂₋₁₀ alkenyl or C₂₋₁₀ alkynyl, wherein each such alkyl, alkenyl or alkynyl group is optionally substituted by one or more groups independently selected from G^{b1}, aryl optionally substituted by one or more groups independently selected from G^{b2}, or heteroaryl optionally substituted by one or more groups independently selected from G^{b3}, or
   one of R^{a1} and R^{a2} may alternatively represent H;
   each of E¹ to E⁶ independently represents halo, ═O, —NR^{b1}R^{b2}, —OR^{b3}, —S(O)ₚR^{b4}, —S(O)_{q}NR^{b5}, —C(O)R^{b6}, —NR^{b7}C(O)R^{b8}, C₁₋₈ alkyl, C₂₋₈ alkenyl or C₂₋₈ alkynyl, wherein each such alkyl, alkenyl or alkynyl group is optionally substituted by one or more groups independently selected from G^{c1}, heterocyclyl optionally substituted by one or more groups independently selected from G^{c2}, or aryl optionally substituted by one or more groups independently selected from G^{c3};
   each of G^{a1} to G^{a5}, G^{b1} to G^{b3}, and G^{c1} to G^{c3} independently represent halo, ═O, —NR^{c1}R^{c2}, —OR^{c3}, —S(O)ₚR^{c4}, —S(O)_{q}NR^{c5}, —C(O)R^{c6}, —NR^{c7}C(O)R^{c8}, C₁₋₈ alkyl, C₂₋₈ alkenyl or C₂₋₈ alkynyl, wherein each such alkyl, alkenyl or alkynyl group is optionally substituted by one or more groups independently selected from W^{a1}, heterocyclyl optionally substituted by one or more groups independently selected from W^{a2}, heteroaryl optionally substituted by one or more groups independently selected from W^{a3}, or aryl optionally substituted by one or more groups independently selected from W^{a4};
   each of R^{b1} to R^{b8}, and R^{c1} to R⁸ independently represents H, C₁₋₈ alkyl, C₂₋₈ alkenyl or C₂₋₈ alkynyl, wherein each such alkyl, alkenyl or alkynyl group is optionally substituted by one or more groups independently selected from W^{b1}, heterocyclyl optionally substituted by one or more groups independently selected from W^{b2}, heteroaryl optionally substituted by one or more groups independently selected from W^{b3}, or aryl optionally substituted by one or more groups independently selected from W^{b4},
   or alternatively any of R^{b1} and R^{b2}, R^{b7} and R^{b8}, R^{c1} and R^{c2}, and R^{c7} and R^{c8} may be linked, together with the atoms to which they are attached, to form a 4- to 6-membered ring, which ring optionally contains one further heteroatom and which ring optionally is substituted by one or more groups independently selected from halo, C₁₋₃ alkyl optionally substituted by one or more halo, and ═O;
   each of W^{a1} to W^{a4}, and W^{b1} to W^{b4} independently represents halo, ═O, —NR^{d1}R^{d2}, —OR^{d3}, —S(O)ₚR^{d4}, —S(O)^{q}NR^{d5}, —C(O)R^{d6}, —NR^{d7}C(O)R^{d8}, C₁₋₈ alkyl, C₂₋₈ alkenyl or C₂₋₈ alkynyl, wherein each such alkyl, alkenyl or alkynyl group is optionally substituted by one or more groups independently selected from Z¹, heterocyclyl optionally substituted by one or more groups independently selected from Z², or aryl optionally substituted by one or more groups independently selected from Z³;
   each R^{d1} to R^{d8} independently represents H, C₁₋₃ alkyl, C₂₋₃ alkenyl or C₂₋₃ alkynyl, wherein each such alkyl, alkenyl or alkynyl group is optionally substituted by one or more fluoro;
   each Z¹ to Z³ independently represents halo, ═O, —NR^{e1}R^{e2}, —OR^{e3}, —S(O)ₚR^{e4}, —S(O)_{q}NR^{e5}, —C(O)R^{e6}, —NR^{e7}C(O)R^{e8};
   each R^{e1} to R^{e8} represents H or C₁₋₃ alkyl, wherein the alkyl group is optionally substituted by one or more fluoro; and
   each p and q independently represents 0, 1 or 2,
   which compounds (including pharmaceutically acceptable salts) may be referred to herein as the “compounds of the first aspect of the invention”.

For the avoidance of doubt, the skilled person will understand that references herein to compounds of particular aspects of the invention (such as the first aspect of the invention, i.e. to compounds of formula I as defined in the first aspect of the invention) will include references to all embodiments and particular features thereof, which embodiments and particular features may be taken in combination to form further embodiments.

Unless indicated otherwise, all technical and scientific terms used herein have the same meaning as commonly understood by one of ordinary skill in the art to which this invention pertains.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Particular acid addition salts that may be mentioned include carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesuphonate, 1- or 2-naphthalene-sulphonate or 1,5-naphthalene-disulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed with alkali metals (such as Na and K salts), alkaline earth metals (such as Mg and Ca salts), organic bases (such as ethanolamine, diethanolamine, triethanolamine, tromethamine and lysine) and inorganic bases (such as ammonia and aluminium hydroxide). More particularly, base addition salts that may be mentioned include Mg, Ca and, most particularly, K and Na salts.

More particular salts that may be mentioned include acetate and trifluoroacetate salts.

For the avoidance of doubt, compounds of the invention may exist as solids, and thus the scope of the invention includes all amorphous, crystalline and part crystalline forms thereof, and may also exist as oils. Where compounds of the first aspect of the invention exist in crystalline and part crystalline forms, such forms may include solvates, which are included in the scope of the invention. Compounds of the first aspect of the invention may also exist in solution.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired optical isomers may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution); for example, with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-z} alkyl groups (where z is the upper limit of the range) defined herein may be straight-chain or, when there is a sufficient number (i.e. a minimum of two or three, as appropriate) of carbon atoms, be branched-chain, and/or cyclic (so forming a C_{3-z} cycloalkyl group). When there is a sufficient number (i.e. a minimum of four) of carbon atoms, such groups may also be part cyclic (so forming a C_{3-z} partial cycloalkyl group). Part cyclic alkyl groups that may be mentioned include cyclopropylmethyl and cyclohexylethyl. When there is a sufficient number of carbon atoms, such groups may also be multicyclic (e.g. bicyclic or tricyclic) or spirocyclic.

Unless otherwise specified, C_{2-z} alkenyl groups (where z is the upper limit of the range) defined herein may be straight-chain or, when there is a sufficient number (i.e. a minimum of three) of carbon atoms, be branched-chain, and/or cyclic (so forming a C_{4-z} cycloalkenyl group). When there is a sufficient number (i.e. a minimum of five) of carbon atoms, such groups may also be part cyclic. Part cyclic alkenyl groups that may be mentioned include cyclopentenylmethyl and cyclohexenylmethyl. When there is a sufficient number of carbon atoms, such groups may also be multicyclic (e.g. bicyclic or tricyclic) or spirocyclic.

Unless otherwise specified, C_{2-z} alkynyl groups (where z is the upper limit of the range) defined herein may be straight-chain or, when there is a sufficient number (i.e. a minimum of four) of carbon atoms, be branched-chain.

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulphur (e.g. oxygen, nitrogen and sulphur).

As used herein, the term heterocyclyl may refer to non-aromatic monocyclic and polycyclic (e.g. bicyclic) heterocyclic groups (which groups may, where containing a sufficient number of atoms, also be bridged) in which at least one (e.g. one to four) of the atoms in the ring system is other than carbon (i.e. a heteroatom), and in which the total number of atoms in the ring system is between three and twelve (e.g. between five and ten and, most preferably, between three and eight, e.g. a 5- or 6-membered heterocyclyl group). Further, such heterocyclyl groups may be saturated, forming a heterocycloalkyl, or unsaturated containing one or more carbon-carbon or, where possible, carbon-heteroatom or heteroatom-heteroatom double and/or triple bonds, forming for example a C_{2-z} (e.g. C_{4-z}) heterocycloalkenyl (where z is the upper limit of the range) or a C_{7-z} heterocycloalkynyl group.

Various heterocyclyl groups will be well-known to those skilled in the art, such as 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, 2,3-dihydroisothiazolyl, dihydropyranyl, dihydropyridinyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, isothiazolidinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuryl, tetrahydropyridinyl (such as 1,2,3,4-tetrahydropyridinyl and 1,2,3,6-tetrahydropyridinyl), thietanyl, thiiranyl, thiolanyl, tetrahydrothiopyranyl, thiomorpholinyl, trithianyl (including 1,3,5-trithianyl), tropanyl and the like.

Substituents on heterocyclyl groups may, where appropriate, be located on any atom in the ring system including a heteroatom. Further, in the case where the substituent is another cyclic compound, then the cyclic compound may be attached through a single atom on the heterocyclyl group, forming a spirocyclic compound. The point of attachment of heterocyclyl groups may be via any atom in the ring system including (where appropriate) a further heteroatom (such as a nitrogen atom), or an atom on any fused carbocyclic ring that may be present as part of the ring system. Heterocyclyl groups may also be in the N- or S-oxidised form.

At each occurrence when mentioned herein, particular heterocyclyl groups that may be mentioned include 3- to 8-membered heterocyclyl groups (e.g. a 4- to 6-membered heterocyclyl group).

For the avoidance of doubt, references to polycyclic (e.g. bicyclic or tricyclic) groups (for example when employed in the context of heterocyclyl or cycloalkyl groups (e.g. heterocyclyl)) will refer to ring systems wherein at least two scissions would be required to convert such rings into a straight chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of alkyl groups) may refer to groups in which the second ring of a two-ring system is formed between two adjacent atoms of the first ring, to groups in which two non-adjacent atoms are linked by an alkylene group (optionally containing one or more heteroatoms), which later groups may be referred to as bridged or to groups in which the second ring is attached to a single atom (i.e. a spiro compound).

Particular heterocyclyl groups that may be mentioned include piperidinyl (e.g. piperidin-1-yl), octahydro-1H-isoindolyl (e.g. octahydro-1H-isoindol-2-yl), azetidinyl (e.g. azetidine-1-yl), oxetanyl (e.g. oxetan-3-yl), morpholinyl (e.g. morpholin-4-yl), piperazinyl (e.g. piperazin-1yl or piperazin-4-yl), azepanyl (e.g. azepan-1-yl), imidazolidinyl (e.g. imidazolidine-2-yl), pyrrolidinyl (e.g. pyrrolidine-1yl), and diazepanyl (e.g. 1,4-diazepan-1-yl).

As may be used herein, the term aryl includes references to C₆₋₁₄ (e.g. C₆₋₁₀) aromatic groups. Such groups may be monocyclic or bicyclic and, when bicyclic, be either wholly or partly aromatic. C₆₋₁₀ aryl groups that may be mentioned include phenyl, naphthyl, 1,2,3,4-tetrahydronaphthyl, indanyl, and the like (e.g. phenyl, naphthyl and the like). Particular aryl groups that may be mentioned include phenyl. For the avoidance of doubt, the point of attachment of substituents on aryl groups may be via any carbon atom of the ring system.

As may be used herein, the term heteroaryl (or heteroaromatic) includes references to 5-to 14-(e.g. 5- to 10-) membered heteroaromatic groups containing one or more heteroatoms selected from oxygen, nitrogen and/or sulfur. Such heteroaryl groups may comprise one, two, or three rings, of which at least one is aromatic. Substituents on heteroaryl/heteroaromatic groups may, where appropriate, be located on any atom in the ring system including a heteroatom.

The point of attachment of heteroaryl/heteroaromatic groups may be via any atom in the ring system including (where appropriate) a heteroatom. Bicyclic heteroaryl/heteroaromatic groups may comprise a benzene ring fused to one or more further aromatic or non-aromatic heterocyclic rings, in which instances, the point of attachment of the polycyclic heteroaryl/heteroaromatic group may be via any ring including the benzene ring or the heteroaryl/heteroaromatic or heterocyclyl ring.

Various heteroaryl groups will be well-known to those skilled in the art, such as pyridinyl, pyrrolyl, furanyl, thiophenyl, oxadiazolyl, thiadiazolyl, thiazolyl, oxazolyl, pyrazolyl, triazolyl, tetrazolyl, isoxazolyl, isothiazolyl, imidazolyl, imidazopyrimidinyl, imidazothiazolyl, thienothiophenyl, pyrimidinyl, furopyridinyl, indolyl, azaindolyl, pyrazinyl, pyrazolopyrimidinyl, indazolyl, pyrimidinyl, quinolinyl, isoquinolinyl, quinazolinyl, benzofuranyl, benzothiophenyl, benzoimidazolyl, benzoxazolyl, benzothiazolyl, benzotriazolyl and purinyl. The oxides of heteroaryl/heteroaromatic groups are also embraced within the scope of the invention (e.g. the N-oxide). As stated above, heteroaryl includes polycyclic (e.g. bicyclic) groups in which one ring is aromatic (and the other may or may not be aromatic). Hence, other heteroaryl groups that may be mentioned include e.g. benzo[1,3]dioxolyl, benzo[1,4]dioxinyl, dihydrobenzo[d]isothiazole, 3,4-dihydrobenz[1,4]oxazinyl, dihydrobenzothiophenyl, indolinyl, 5H,6H,7H-pyrrolo[1,2-b]pyrimidinyl, 1,2,3,4-tetrahydroquinolinyl, thiochromanyl and the like.

Particular heteroaryl groups that may be mentioned include 1,3,4-oxadiazolyl (e.g. 1,3,4-oxadiazol-2-yl).

For the avoidance of doubt, where a ring is depicted having circle therein (for example, as with the ring formed from X¹ to X⁵ in compounds described herein, such as in formula I), its presence shall indicate that the relevant ring is aromatic (in the case of the ring formed from X¹ to X⁵, so forming a heteroaryl group).

For the avoidance of doubt, as used herein, references to heteroatoms will take their normal meaning as understood by one skilled in the art. Particular heteroatoms that may be mentioned include phosphorus, selenium, tellurium, silicon, boron, oxygen, nitrogen and sulfur (e.g. oxygen, nitrogen and sulphur).

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. For example, in the situation in which two or more G^{c2} groups are present, those G^{c2} groups may be the same or different. Similarly, where two or more G^{c2} groups are present and each represent —OR^{c3}, the —OR^{c3} groups in question may be the same or different.

Also for the avoidance of doubt, when a term such as “G^{a1} to G^{a5}” is employed herein, this will be understood by the skilled person to mean G^{a1}, G^{a2}, G^{a3}, G^{a4} and G^{a5}, inclusively. Unless otherwise stated, the same reasoning will apply to other such terms used herein.

Further for the avoidance of doubt, when it is specified that a substituent is itself optionally substituted by one or more substituents (e.g. C₂₋₈ alkyl optionally substituted by one or more groups independently selected from G^{a1}), these substituents where possible may be positioned on the same or different atoms.

The skilled person will appreciate that compounds of the invention that are the subject of this invention include those that are stable. That is, compounds of the invention include those that are sufficiently robust to survive isolation, e.g. from a reaction mixture, to a useful degree of purity.

For the avoidance of doubt, where groups are referred to herein as being optionally substituted it is specifically contemplated that such optional substituents may not be present, in which case the optionally substituted group may be referred to as being unsubstituted.

In particular embodiments (i.e. particular embodiments of the first aspect of the invention), the compound of formula I is such that:

X¹ and X⁴ represent C; and
any one to three of X², X³ and X⁵ represents a heteroatom selected from N, O and S, with the proviso that only one of X², X³ and X⁵ may represent O or S.

In more particular embodiments:

X¹ and X⁴ represent C;
X² represents N;
X³ represents N or CR⁵ (such as wherein R⁵ represents H); and
X⁵ represents O.

In yet more particular embodiments:

X¹ and X⁴ represent C;
X² represents N;
X³ represents N; and
X⁵ represents O.

In alternative embodiments:

X¹ and X⁴ represent C.
X² represents N,
X³ represents N, and
X⁵ represents O;
or
X¹ and X⁵ represent C (i.e. where X¹ represents C and X⁵ represents CR⁵), and
X² to X⁴ represent N;
or
X¹ and X⁴ represent C,
X³ and X⁵ represent N, and
X² represents O;
or
X¹ and X⁴ represent C,
X² represents C (i.e. CR⁵),
X³ represents O, and
X⁵ represents N;
or
X¹ and X⁴ represent C,
X² represents C (i.e. CR⁵),
X³ represents N, and
X⁵ represents S;
or
X¹ and X⁴ represent C,
X⁵ represents C (i.e. CR⁵),
X² represents N, and
X³ represents O;
or
X¹ and X⁴ represent C,
X⁵ represents C (i.e. CR⁵),
X² represents O, and
X³ represents N.

In certain embodiments, where R¹ represents halo, the halo is bromo or chloro (e.g. chloro).

In particular embodiments, R¹ represents

(i) heteroaryl optionally substituted by one or more groups selected from E¹, or heterocyclyl optionally substituted by one or more groups independently selected from E², such as wherein each such heteroaryl or heterocyclyl group is attached via a constituent heteroatom,
(ii) —NR^{a1}R^{a2}, —OR^{a3}, —S(O)ₚR^{a4} or —S(O)_{q}NR^{a5},
(iii) C₁₋₁₀ alkyl, C₂₋₁₀ alkenyl or C₂₋₁₀ alkynyl, wherein each such alkyl, alkenyl or alkynyl group is optionally substituted by one or more groups independently selected from E³, or
(iv) aryl optionally substituted by one or more groups independently selected from E⁴.

In more particular embodiments, R¹ represents:

(i) heteroaryl optionally substituted by one or more groups selected from E¹, or heterocyclyl optionally substituted by one or more groups independently selected from E², such as wherein each such heteroaryl or heterocyclyl group is attached via a constituent heteroatom (e.g. a constituent N atom),
(ii) —NR^{a1}R^{a2}, —OR^{a3}, —S(O)ₚR^{a4} or —S(O)_{q}NR^{a5}, or
(iii) C₁₋₁₀ alkyl, C₂₋₁₀ alkenyl or C₂₋₁₀ alkynyl, wherein each such alkyl, alkenyl or alkynyl group is optionally substituted by one or more groups independently selected from E³.

As used herein, references to groups, such as groups representing R¹, being attached via a constituent heteroatom, will refer to the point of attachment of such groups (i.e. the point of attachment of the group, such as R¹, to the core of the molecule; namely, the essential purine-derived core moiety, as depicted in formula I) being at a heteroatom that forms part of the relevant group (i.e. forming a part of the group as defined, such as the group as defined for R¹, rather than any substituent thereon). For the avoidance of doubt, the skilled person will understand that, in order to form the point of attachment and also form part of the relevant group, such heteroatoms must have an appropriate valency (e.g. a valency of at least 3). Thus, it is particularly noted that in circumstances referring to such groups being attached via a constituent heteroatom that heteroatom may be N (i.e. a N forming part of the group being available to form a suitable bond).

In certain embodiments, R¹ represents:

(i) heteroaryl optionally substituted by one or more groups selected from E¹, or heterocyclyl optionally substituted by one or more groups independently selected from E², such as wherein each such heteroaryl or heterocyclyl group is attached via a constituent heteroatom, or
(ii) —NR^{a1}R^{a2}, —OR^{a3}, —S(O)ₚR^{a4} or —S(O)_{q}NR^{a5}, such as wherein p represents 0 or 2 and/or q represents 2.

In alternative embodiments, R¹ represents:

(i) heteroaryl optionally substituted by one or more groups selected from E¹, or heterocyclyl optionally substituted by one or more groups independently selected from E², such as wherein each such heteroaryl or heterocyclyl group is attached via a constituent carbon atom,
(ii) C₁₋₁₀ alkyl, C₂₋₁₀ alkenyl or C₂₋₁₀ alkynyl, wherein each such alkyl, alkenyl or alkynyl group is optionally substituted by one or more groups independently selected from E³, or
(iii) aryl optionally substituted by one or more groups independently selected from E⁴.

In further alternative embodiments, R¹ represents:

(i) C₁₋₁₀ alkyl, C₂₋₁₀ alkenyl or C₂₋₁₀ alkynyl, wherein each such alkyl, alkenyl or alkynyl group is optionally substituted by one or more groups independently selected from E³, or
(ii) aryl optionally substituted by one or more groups independently selected from E⁴.

In yet further alternative embodiments, R¹ represents

(i) C₁₋₁₀ alkyl, C₂₋₁₀ alkenyl or C₂₋₁₀ alkynyl, wherein each such alkyl, alkenyl or alkynyl group is optionally substituted by one or more groups independently selected from E³.

In particular embodiments, R¹ represents:

(i) heterocyclyl optionally substituted by one or more groups independently selected from E², such as wherein each such heteroaryl or heterocyclyl group is attached via a constituent heteroatom (e.g. a constituent N atom),
(ii) —NR^{a1}R^{a2}, —OR^{a3}, —S(O)ₚR^{a4} or —S(O)_{q}NR^{a5}, wherein p represents 0 or 2, or
(iii) C₁₋₁₀ alkyl (e.g. C₁₋₆ alkyl, such as C₁₋₃ alkyl), optionally substituted by one or more groups independently selected from E³.

In more particular embodiments, R¹ represents:

(i) heterocyclyl optionally substituted by one or more groups independently selected from E², particularly wherein the heterocyclyl group is attached via a constituent heteroatom (e.g. a constituent N atom); or
(ii) —NR^{a1}R^{a2}, —OR^{a3}, —S(O)ₚR^{a4}, wherein p represents 0 or 2.

In alternative embodiments, R¹ represents:

(i) heterocyclyl optionally substituted by one or more groups independently selected from E², particularly wherein the heterocyclyl group is attached via a constituent heteroatom (e.g. a constituent N atom);
(ii) —NR^{a1}R^{a2}, —OR^{a3}, —S(O)ₚR^{a4}, wherein p represents 0 or 2 (e.g. 0); or (iii) halo (e.g. chloro).

In yet more particular embodiments, R¹ represents heterocyclyl optionally substituted by one or more groups independently selected from E², particularly wherein the heterocyclyl group is attached via a constituent heteroatom.

In alternative embodiments, R¹ represents C₁₋₁₀ alkyl (e.g. C₁₋₆ alkyl, such as C₁₋₃ alkyl), optionally substituted by one or more groups independently selected from E³.

In further alternative embodiments, R¹ represents halo (e.g. chloro).

In particular embodiments that may be mentioned, R^{a4} does not represent H.

In more particular embodiments that may be mentioned:

R^{a1} represents H, C₁₋₁₀ alkyl (such as C₁₋₆ alkyl) or C₂₋₁₀ alkenyl (such as C₂₋₆ alkenyl), each optionally substituted with one or more (e.g. one) groups independently selected from G^{b1};
R^{a2} represents C₁₋₁₀ alkyl (such as C₁₋₆ alkyl) or C₂₋₁₀ alkenyl (such as C₂₋₆ alkenyl), each optionally substituted with one or more (e.g. one) groups independently selected from G^{b1};
R^{a3} represents C₁₋₁₀ alkyl (such as C₁₋₆ alkyl), optionally substituted with one or more (e.g. one) groups independently selected from G^{b1}; and/or (e.g. and)
R^{a4} represents C₁₋₁₀ alkyl (such as C₁₋₆ alkyl), optionally substituted with one or more (e.g. one) groups independently selected from G^{b1}.

For example, in particular embodiments where R¹ may represent —NR^{a1}R^{a2}:

R^{a1} may present H, C₁₋₃ alkyl optionally substituted with one or more group selected from G^{b1}, or C₂₋₃ alkenyl;
R^{a2} may present C₁₋₃ alkyl optionally substituted with one or more group selected from G^{b1}, or C₂₋₃ alkenyl; and
G^{b1} may represent aryl (e.g. phenyl) or —OR³, particularly wherein R^{c3} represents H.

In particular embodiments where R¹ may represent —OR^{a3}:

R^{a3} may present C₁₋₃ alkyl (e.g. methyl).

In particular embodiments where R¹ may represent —S(O)ₚR^{a4}:

where p represents 0, R^{a4} may represent C₁₋₆ alkyl (e.g. ethyl or cyclohexyl); and
where p represents 2, R^{a4} may represent C₁₋₆ alkyl (e.g. ethyl or cyclohexyl).

In yet more particular embodiments, where R¹ represents heterocyclyl, the heterocyclyl group may:

(a) be saturated; and/or (e.g. and)
(b) comprise 4 to 8 atoms (e.g. including 1 or 2 heteroatoms).

In particular embodiments, where R¹ represents heterocyclyl, the heterocyclyl may be selected from:

piperidinyl (e.g. piperidin-1-yl), octahydro-1H-isoindolyl (e.g. octahydro-1H-isoindol-2-yl), azetidinyl (e.g. azetidine-1-yl), morpholinyl (e.g. morpholin-4-yl), piperazinyl (e.g. piperazin-1yl or piperazin-4-yl), azepanyl (e.g. azepan-1-yl), pyrrolidinyl (e.g. pyrrolidine-1yl), and diazepanyl (e.g. 1,4-diazepan-1-yl),
optionally substituted by one or more (e.g. one or two) groups selected from E².

In particular, where R¹ represents heterocyclyl, the heterocyclyl may be selected from:

piperidinyl (e.g. piperidin-1-yl), azetidinyl (e.g. azetidine-1-yl), and piperazinyl (e.g. piperazin-1yl or piperazin-4-yl),
optionally substituted by one or more (e.g. one or two) groups selected from E².

More particularly, where R¹ represents heterocyclyl, the heterocyclyl may be piperazinyl (e.g. piperazin-1yl or piperazin-4-yl) optionally substituted by one or more (e.g. one or two) groups selected from E².

As described herein, heterocyclyl groups representing R¹ may be optionally substituted by one or more (e.g. one or two) groups selected from E².

In particular embodiments, each E² group, where present, may represent:

halo (such as F);
—NR^{b1}R^{b2};

### —OR^{b3}.

C₁₋₈ alkyl (such as C₁₋₆ alkyl) optionally substituted by one or more groups independently selected from G^{c1};
heterocyclyl (such as oxetanyl, e.g. oxetan-3-yl, or imidazolidinyl, e.g. imidazolidine-2-yl) 25 optionally substituted by one or more (e.g. one or two) groups independently selected from G^{c2}; or
aryl (such as phenyl) optionally substituted by one or more groups independently selected from G^{c3}.

In particular embodiments:

R^{b1} and R^{b2} may independently represent H or C₁₋₃ alkyl (e.g. methyl) optionally substituted by one or more group selected from ═O and —O^{t}Bu (for example, such methyl groups may be substituted with both ═O and —O^{t}Bu, so forming a —C(O)O^{t}Bu group, which also be referred to as a Boc group);
R^{b3} may represent H or C₁₋₃ alkyl (e.g. methyl);
G^{c1} may represent ═O, —NR^{c1}R^{c2} or —OR^{c3}, particularly where R^{c1} and R^{c2} represent H and/or (e.g. and) R^{c3} represents H or C₁₋₄ alkyl (e.g. methyl or Bu); and/or (e.g. and)
G^{c2} may represent ═O.

For example, where and E² group represents C₁₋₈ alkyl (such as C₁₋₆ alkyl) optionally substituted by one or more groups independently selected from G^{c1}, such alkyl groups may be a C₁ alkyl (i.e. a methyl) substituted by ═O and —O^{t}Bu, so forming a Boc group.

The skilled person will understand that where substituents are formed from C₁ alkyl substituted by ═O and —O^{t}Bu, so forming a Boc group, such substituents may be present, in particular, on a N (such as a N which is a constituent of a heterocycyl or a N of an amino group, e.g. a heterocyclyl group representing R¹), so forming a NBoc moiety.

In particular embodiments, each E³ group, where present, may represent aryl (e.g. phenyl) optionally substituted by one or more groups independently selected from G^{c3}.

In particular embodiments that may be mentioned, R¹ is selected from the following groups:

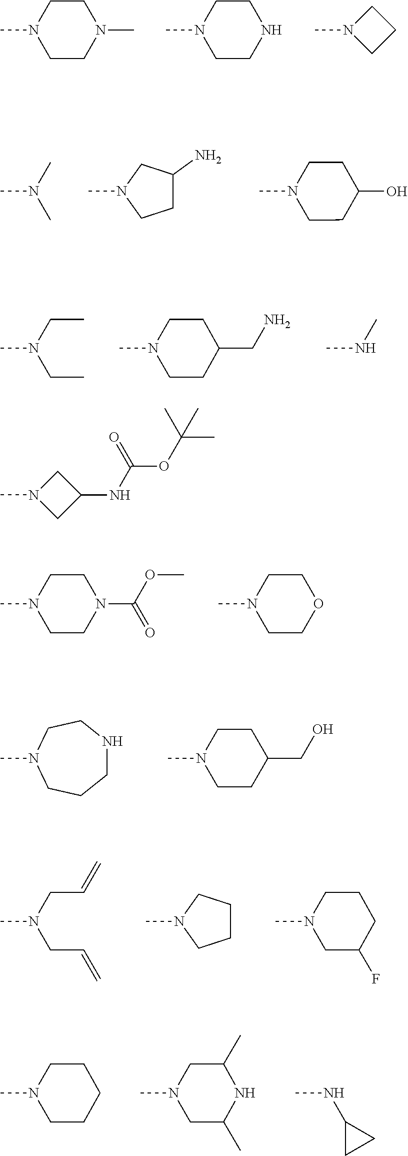

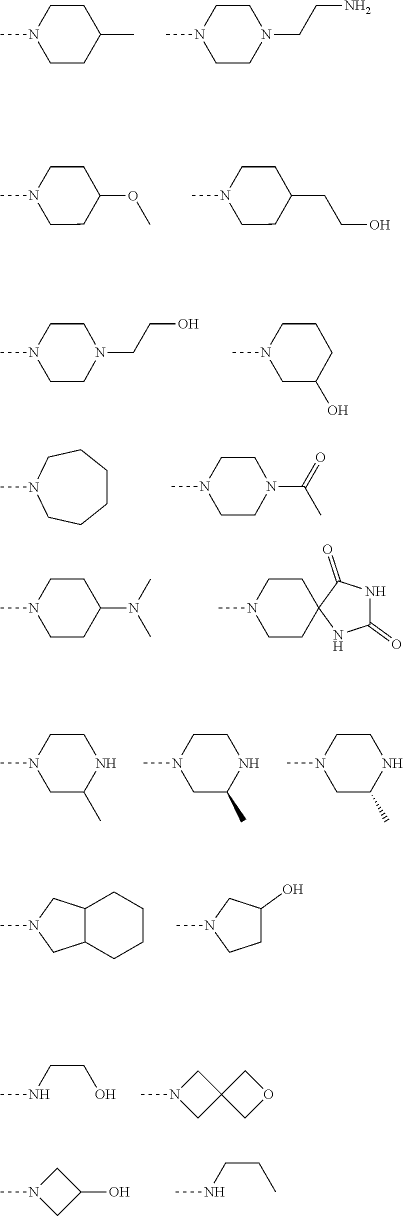

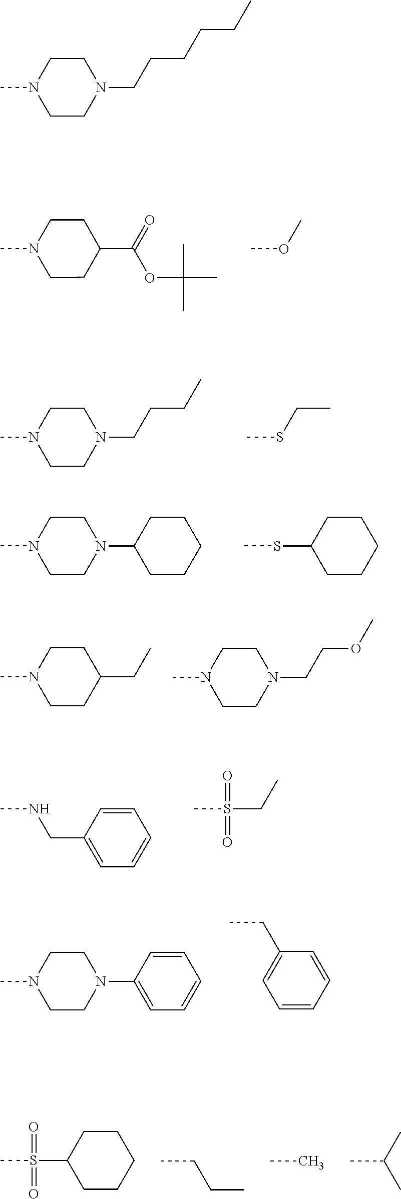

wherein the dashed bond (i.e. “--”) indicates the position of attachment (i.e. the point of attachment of R¹ to the core of the molecule; namely, the essential purine-derived core moiety, as depicted in formula I).

In more particular embodiments that may be mentioned, R¹ is selected from the following groups:

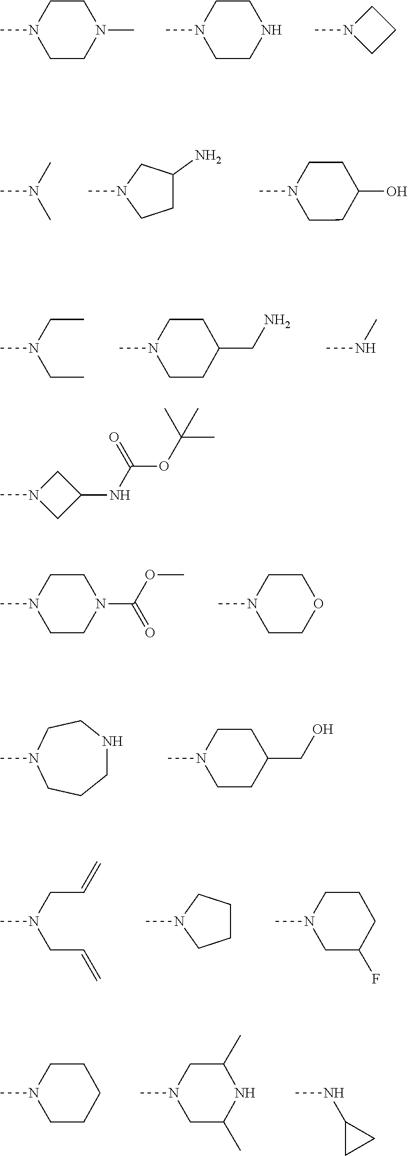

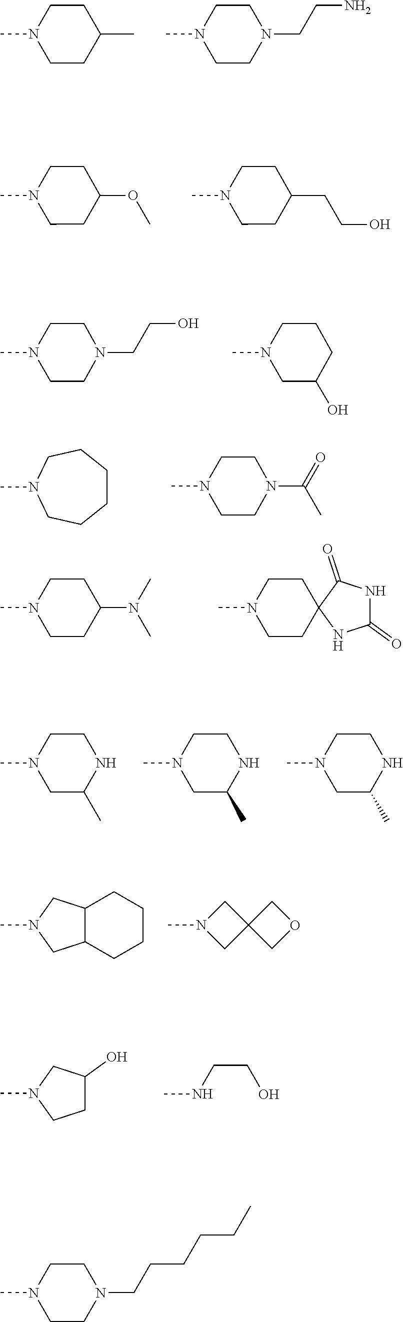

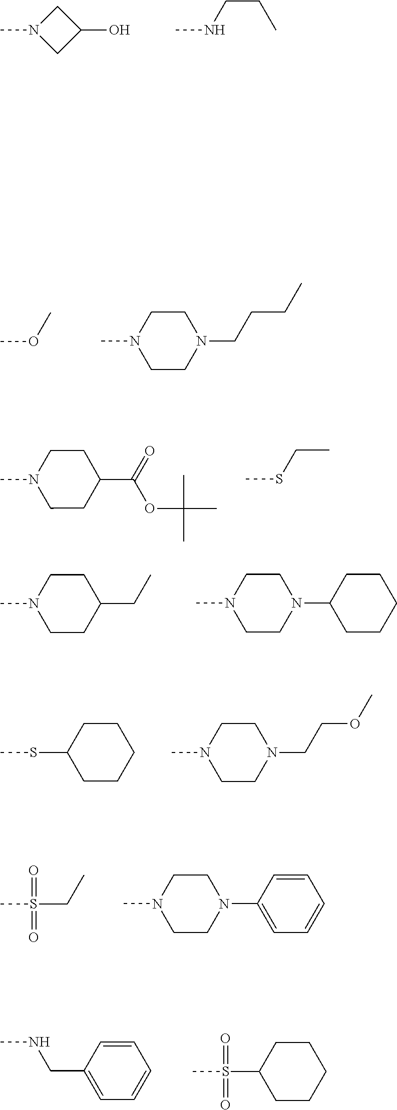

wherein the dashed bond (i.e. “--”) indicates the position of attachment (i.e. the point of attachment of R¹ to the core of the molecule; namely, the essential purine-derived core moiety, as depicted in formula I).

In alternative embodiments that may be mentioned, R¹ is selected from the following groups:

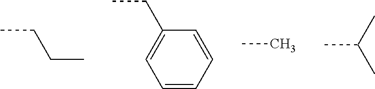

wherein the dashed bond (i.e. “--”) indicates the position of attachment (i.e. the point of attachment of R¹ to the core of the molecule; namely, the essential purine-derived core moiety, as depicted in formula I).

In further embodiments that may be mentioned, R¹ is (or is also, i.e. in addition to the other embodiments provided) selected from the following groups:

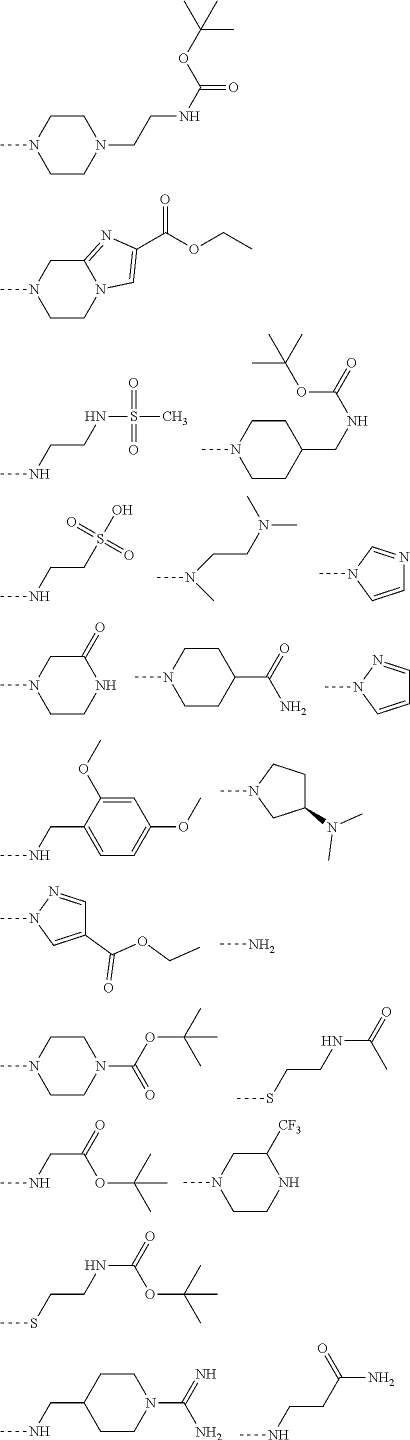

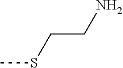

In particular embodiments, R² and R³ each independently represent C₁₋₄ alkyl optionally substituted by one or more (e.g. one or two, such as one) groups independently selected from Ga.

In more particular embodiments, R² and R³ each independently represent C₁₋₃ alkyl (e.g. C₁ alkyl) optionally substituted by one or more (e.g. one) groups independently selected from G^{a1}.

In yet more particular embodiments, R² and R³ each represent methyl.

In particular embodiments, R⁴ represents aryl optionally substituted by one or more (e.g. one or two) groups independently selected from E⁵.

In more particular embodiments, R⁴ represents phenyl optionally substituted by one or more (e.g. one or two) groups independently selected from E⁵.

In alternative embodiments that may be mentioned, R⁴ represents:

phenyl optionally substituted by one or more (e.g. one or two) groups independently selected from E⁵; or
pyridinyl (e.g. pyridine-3-yl), indolyl (e.g. indol-6-yl) or indazolyl (e.g. indazol-5-yl) optionally substituted by one or more (e.g. one or two) groups independently selected from E⁶.

Thus, in particular embodiments that may be mentioned, the compound of formula I is a compound of formula Ia

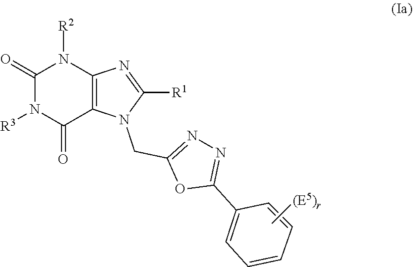

wherein R¹, R², R³ and E⁵ are as described herein (i.e. for compounds of the first aspect of the invention, including all embodiments thereof), and wherein r represents 0 to 5.

In particular such embodiments, r represents 0 to 3 (e.g. 0, 1 or 2, such as 1 or 2).

For example, in particular embodiments that may be mentioned, R⁴ may be represented as follows:

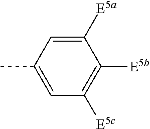

wherein the dashed bond (i.e. “--”) indicates the position of attachment (i.e. the point of attachment of the R⁴ group to the core of the molecule; namely, the essential purine-derived core moiety, as depicted in formula I), and wherein each of E^{5a}, E^{5b} and E^{5c} represents an E⁵ group as defined herein.

In particular embodiments, each E⁵ independently represents halo, or C₁₋₈ alkyl optionally substituted by one or more (e.g. one or two, such as one) groups independently selected from G^{c1}.

In more particular embodiments, each E⁵ independently represents halo (e.g. chloro), or C₁₋₃ alkyl (e.g. C₁ alkyl) optionally substituted by one or more (e.g. one) groups independently selected from G^{c1}.

In yet more particular embodiments, each E⁵ independently represents chloro or methyl.

In particular embodiments that may be mentioned, R⁴ is selected from the following groups:

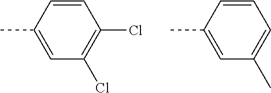

wherein the dashed bond (i.e. “--”) indicates the position of attachment (i.e. the point of attachment of the R⁴ group to the core of the molecule; namely, the essential purine-derived core moiety, as depicted in formula I).

In further embodiments that may be mentioned, R⁴ is (or is also, i.e. in addition to the other embodiments provided) selected from the following groups:

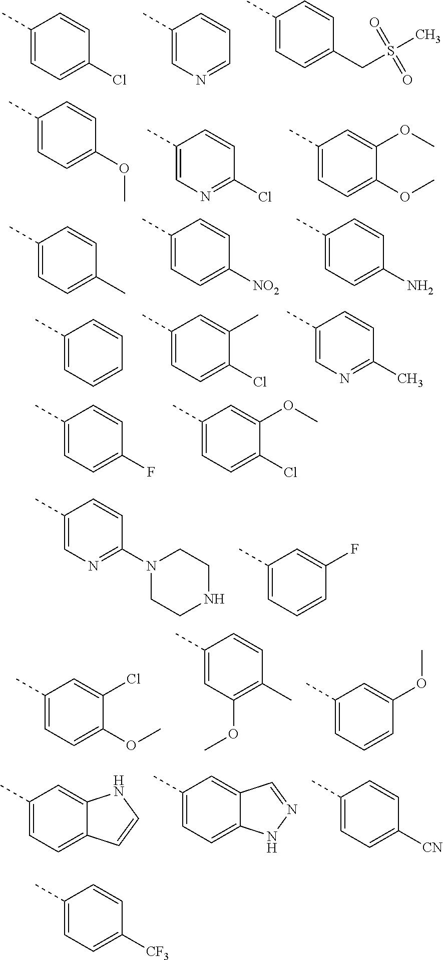

For the avoidance of doubt, particular R⁵ groups that may be mentioned include H.

As described herein, in particular embodiments that may be mentioned, R¹ is a group wherein the point of attachment is via a N (i.e. a group selected from the NR^{a1}R^{a2}, heterocyclyl and heteroaryl groups representing R¹ as described herein, wherein the point of attachment of the heterocyclyl and heteroaryl groups is via a N).

In more particular embodiments that may be mentioned, R¹ represents NR^{a1}R^{a2} or heterocyclyl, as described herein, wherein the point of attachment of the heterocyclyl is via a N atom.

Thus, in particular embodiments, R¹ may be alternatively defined as a group of structure —N(Q¹)Q², wherein:

Q¹ and Q² represent a group as defined herein for R^{a1} and R^{a2}, respectively (i.e. for R^{a1} and R^{a2} as defined in the context of R¹), or
Q¹ and Q² are linked to form, together with the N to which they are attached, heterocyclyl optionally substituted by one or more groups independently selected from E², as defined herein for R¹.

In more particular embodiments, the compound of formula I is a compound of formula Ib

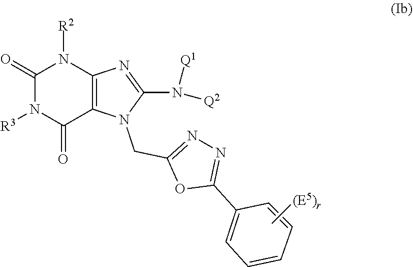

wherein R², R³, E⁵ and r are as defined herein, and wherein:
Q¹ and Q² represent a group as defined herein for R^{a1} and R^{a2}, respectively (i.e. for R^{a1} and R^{a2} as defined in the context of R¹), or
Q¹ and Q² are linked to form, together with the N to which they are attached, heterocyclyl optionally substituted by one or more groups independently selected from E², as defined herein for R¹ (i.e. in compounds of formula I and all embodiments thereof).

In certain embodiments that may be mentioned, where R¹ represents —NR^{a1}R^{a2}R^{a2} represents H.

In certain embodiments that may be mentioned, where R¹ represents heterocyclyl, that heterocyclyl is a group of the following formula

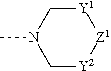

wherein the dashed bond indicates the point of attachment, and wherein:
Y¹ and Y² either both represent a direct bond, or both represent —CR⁶(R⁷)—; and
Z¹ represents a group selected from —CR⁶(R⁷)—, —N(R⁸)— and —O— (e.g. selected from —CR⁶(R⁷)— and —N(R⁸)—),
with the proviso that where Y¹ and Y² represent a direct bond Z represents —CR⁶(R⁷)—,
wherein each of R⁶ to R⁸ independently represents H or E², as defined herein,
such as (i.e. optionally) wherein only up to (i.e. a maximum of) one E² group may be present (such as wherein one E² group is present).

In particular embodiments that may be mentioned, each E² represents —NR^{b1}R^{b2} (particularly wherein R^{b1} and R^{b2} represent H) or C₁₋₂ alkyl, wherein the C₁₋₂ alkyl group is optionally substituted with one or more (e.g. one) G^{c1} (such as wherein G^{c1} represents —NR^{c1}R^{c2}, particularly wherein R^{c1} and R^{c2} represent H).

For the avoidance of doubt, unless otherwise specified, p may in particular represent 0 or 2 (e.g. 2) and q may in particular represent 2.

In an alternative first embodiment of the invention that may be mentioned, there is provided a compound of formula I as defined herein but wherein:

each of E¹ to E⁶ independently represents halo, ═O, —NR^{b1}R^{b2}, —OR^{b3}, —S(O)ₚR^{b4}, —S(O)_{q}NR^{b5}, —C(O)Rb, —NR^{b7}C(O)R^{b8}, —CN, —NO₂, —C(═NR^{b9})NR^{b10}R^{b11}, C₁₋₈ alkyl, C₂₋₈ alkenyl or C₂₋₈ alkynyl, wherein each such alkyl, alkenyl or alkynyl group is optionally substituted by one or more groups independently selected from G^{c1}, heterocyclyl optionally substituted by one or more groups independently selected from G^{c2}, or aryl optionally substituted by one or more groups independently selected from G^{c3};
each of G^{a1} to G^{a5}, G^{b1} to G^{b3}, and G^{c1} to G^{c3} independently represent halo, ═O, —NR^{c1}R^{c2}, —OR^{c3}, —S(O)ₚR^{c4}, —S(O)_{q}NR^{c5}, —C(O)R^{c6}, —NR^{c7}C(O)R^{c8}, —CN, —NO₂, —C(═NR^{c9})NR^{c10}R^{c11}, C₁₋₈ alkyl, C₂₋₈ alkenyl or C₂₋₈ alkynyl, wherein each such alkyl, alkenyl or alkynyl group is optionally substituted by one or more groups independently selected from W^{a1}, heterocyclyl optionally substituted by one or more groups independently selected from W^{a2}, heteroaryl optionally substituted by one or more groups independently selected from W^{a3}, or aryl optionally substituted by one or more groups independently selected from W^{a4};
each of R^{b1} to R^{b11}, and R^{c1} to R^{c11} independently represents H, C₁₋₈ alkyl, C₂₋₈ alkenyl or C₂₋₈ alkynyl, wherein each such alkyl, alkenyl or alkynyl group is optionally substituted by one or more groups independently selected from W^{b1}, heterocyclyl optionally substituted by one or more groups independently selected from W^{b2}, heteroaryl optionally substituted by one or more groups independently selected from W^{b3}, or aryl optionally substituted by one or more groups independently selected from W⁴,
or alternatively any of R^{b1} and R^{b2}, R^{b7} and R^{b8}, R^{c1} and R^{c2}, and R^{c7} and R^{c8} may be linked, together with the atoms to which they are attached, to form a 4- to 6-membered ring, which ring optionally contains one further heteroatom and which ring optionally is substituted by one or more groups independently selected from halo, C₁₋₃ alkyl optionally substituted by one or more halo, and ═O;
each of W^{a1} to W^{a4}, and W^{b1} to W^{b4} independently represents halo, ═O, —NR^{d1}R^{d2}, —OR^{d3}, —S(O)ₚR^{d4}, —S(O)_{q}NR^{d5}, —C(O)R^{d6}, —NR^{d7}C(O)R^{d8}, —CN, —NO₂, —C(═NR^{d9})NR^{d10}R^{d11}, C₁₋₈ alkyl, C₂₋₈ alkenyl or C₂₋₈ alkynyl, wherein each such alkyl, alkenyl or alkynyl group is optionally substituted by one or more groups independently selected from Z¹, heterocyclyl optionally substituted by one or more groups independently selected from Z², or aryl optionally substituted by one or more groups independently selected from Z³;
each R^{d1} to R^{d11} independently represents H, C₁₋₃ alkyl, C₂₋₃ alkenyl or C₂₋₃ alkynyl, wherein each such alkyl, alkenyl or alkynyl group is optionally substituted by one or more fluoro;
each Z¹ to Z³ independently represents halo, ═O, —NR^{e1}R^{e2}, —OR^{e3}, —S(O)ₚR^{e4}, —S(O)^{q}NR^{e5}, —C(O)R^{e6}, —NR^{e7}C(O)R^{e8}, —CN, —NO₂ or —C(═NR^{e9})NR^{e10}R^{e11}; and
each R^{e1} to R^{e11} represents H or C₁₋₃ alkyl, wherein the alkyl group is optionally substituted by one or more fluoro.

In particular such embodiments, R^{b9} to R^{b11}, R^{c9} to R^{c11}, R^{d9} to R^{d11}, and R^{e9} to R^{e11} each represent H.

In particular embodiments that may be mentioned (particularly wherein R¹ represents halo, such as wherein R¹ represents Cl), there is the proviso that the compound of formula I (or, similarly, the compound of formula II as described herein) is not a compound selected from the list consisting of:
8-chloro-7-[(2-cyclopropyl-5-oxazolyl)methyl]-3,7-dihydro-1,3-dimethyl-1H-purine-2,6-dione;
8-chloro-3,7-dihydro-1,3-dimethyl-7-[[2-(3-thienyl)-4-thiazolyl]methyl]-1H-purine-2,6-dione;
8-chloro-3,7-dihydro-1,3-dimethyl-7-[(1-phenyl-1H-1,2,4-triazol-3-yl)methyl]-1H-purine-2,6-dione;
8-chloro-7-[[2-(2-fluorophenyl)-4-thiazolyl]methyl]-3,7-dihydro-1,3-dimethyl-1H-purine-2,6-dione;
8-chloro-3,7-dihydro-7-[[2-(4-methoxyphenyl)-4-oxazolyl]methyl]-1,3-dimethyl-1H-purine-2,6-dione;
8-chloro-3,7-dihydro-1,3-dimethyl-7-[[5-(2-thienyl)-2-oxazolyl]methyl]-1H-purine-2,6-dione;
8-chloro-7-[(5-cyclopropyl-1,2,4-oxadiazol-3-yl)methyl]-3,7-dihydro-1,3-dimethyl-1H-purine-2,6-dione;
4-[4-[(8-chloro-1,2,3,6-tetrahydro-1,3-dimethyl-2,6-dioxo-7H-purin-7-yl)methyl]-2-thiazolyl]-benzamide;
4-[4-[(8-chloro-1,2,3,6-tetrahydro-1,3-dimethyl-2,6-dioxo-7H-purin-7-yl)methyl]-2-thiazolyl]-methylbenzoate;
7-[[2-(1,3-benzodioxol-5-yl)-4-thiazolyl]methyl]-8-chloro-3,7-dihydro-1,3-dimethyl-1H-purine-2,6-dione;
8-chloro-7-[[2-(3-furanyl)-4-thiazolyl]methyl]-3,7-dihydro-1,3-dimethyl-1H-purine-2,6-dione;
8-chloro-3,7-dihydro-1,3-dimethyl-7-[(5-methyl-2-phenyl-4-oxazolyl)methyl]-1H-purine-2,6-dione;
8-chloro-3,7-dihydro-1,3-dimethyl-7-[(3-phenyl-1,2,4-oxadiazol-5-yl)methyl]-1H-purine-2,6-dione;
8-chloro-3,7-dihydro-1,3-dimethyl-7-[(5-phenyl-3-isoxazolyl)methyl]-1H-purine-2,6-dione;
8-chloro-3,7-dihydro-1,3-dimethyl-7-[[3-(3-thienyl)-1,2,4-oxadiazol-5-yl]methyl]-1H-purine-2,6-dione;
8-chloro-3,7-dihydro-1,3-dimethyl-7-[[3-(4-nitrophenyl)-1,2,4-oxadiazol-5-yl]methyl]-1H-purine-2,6-dione;
8-chloro-3,7-dihydro-7-[[2-(4-methoxyphenyl)-4-thiazolyl]methyl]-1,3-dimethyl-1H-purine-2,6-dione;
8-chloro-7-[[2-(4-chlorophenyl)-4-oxazolyl]methyl]-3,7-dihydro-1,3-dimethyl-1H-purine-2,6-dione;
8-chloro-7-[[3-(2,4-dimethoxyphenyl)-1,2,4-oxadiazol-5-yl]methyl]-3,7-dihydro-1,3-dimethyl-1H-purine-2,6-dione;
8-chloro-3,7-dihydro-7-[[2-(2-methoxyphenyl)-4-thiazolyl]methyl]-1,3-dimethyl-1H-purine-2,6-dione;
8-chloro-3,7-dihydro-1,3-dimethyl-7-[[5-(3-thienyl)-1,2,4-oxadiazol-3-yl]methyl]-1H-purine-2,6-dione;
8-chloro-7-[[2-(4-fluorophenyl)-4-oxazolyl]methyl]-3,7-dihydro-1,3-dimethyl-1H-purine-2,6-dione;
8-chloro-7-[[2-(2-chlorophenyl)-4-thiazolyl]methyl]-3,7-dihydro-1,3-dimethyl-1H-purine-2,6-dione;
8-chloro-7-[[2-(3,4-dimethoxyphenyl)-4-thiazolyl]methyl]-3,7-dihydro-1,3-dimethyl-1H-purine-2,6-dione;
8-chloro-7-[[5-(2-chlorophenyl)-1,3,4-oxadiazol-2-yl]methyl]-3,7-dihydro-1,3-dimethyl-1H-purine-2,6-dione;
8-chloro-3,7-dihydro-1,3-dimethyl-7-[[2-(2-thienyl)-4-thiazolyl]methyl]-1H-purine-2,6-dione;
8-chloro-3,7-dihydro-1,3-dimethyl-7-[[3-(3-nitrophenyl)-1,2,4-oxadiazol-5-yl]methyl]-1H-purine-2,6-dione;
7-[[3-(3-bromophenyl)-1,2,4-oxadiazol-5-yl]methyl]-8-chloro-3,7-dihydro-1,3-dimethyl-1H-purine-2,6-dione;
8-chloro-3,7-dihydro-1,3-dimethyl-7-[[3-[4-(trifluoromethyl)phenyl]-1,2,4-oxadiazol-5-yl]methyl]-1H-purine-2,6-dione;
8-chloro-7-[[3-[4-(difluoromethoxy)-3-methoxyphenyl]-1,2,4-oxadiazol-5-yl]methyl]-3,7-dihydro-1,3-dimethyl-1H-purine-2,6-dione;
8-chloro-3,7-dihydro-1,3-dimethyl-7-[(2-phenyl-4-thiazolyl)methyl]-1H-purine-2,6-dione;
8-chloro-7-[[3-(4-ethylphenyl)-1,2,4-oxadiazol-5-yl]methyl]-3,7-dihydro-1,3-dimethyl-1H-purine-2,6-dione;
8-chloro-7-[[2-(2,4-dimethoxyphenyl)-4-thiazolyl]methyl]-3,7-dihydro-1,3-dimethyl-1H-purine-2,6-dione;
8-chloro-7-[[5-(4-fluorophenyl)-2-oxazolyl]methyl]-3,7-dihydro-1,3-dimethyl-1H-purine-2,6-dione;
8-chloro-7-[[5-(4-chlorophenyl)-1,2,4-oxadiazol-3-yl]methyl]-3,7-dihydro-1,3-dimethyl-1H-purine-2,6-dione;
8-chloro-3,7-dihydro-1,3-dimethyl-7-[[2-(2-pyridinyl)-4-thiazolyl]methyl]-1H-purine-2,6-dione;
8-chloro-3,7-dihydro-1,3-dimethyl-7-[[5-(2-thienyl)-1,2,4-oxadiazol-3-yl]methyl]-1H-purine-2,6-dione;
8-chloro-7-[[5-(2-chlorophenyl)-1,2,4-oxadiazol-3-yl]methyl]-3,7-dihydro-1,3-dimethyl-1H-purine-2,6-dione;
8-chloro-3,7-dihydro-1,3-dimethyl-7-[[3-[4-(1-methylethyl)phenyl]-1,2,4-oxadiazol-5-yl]methyl]-1H-purine-2,6-dione;
8-chloro-7-[[2-(4-ethylphenyl)-4-thiazolyl]methyl]-3,7-dihydro-1,3-dimethyl-1H-purine-2,6-dione;
8-chloro-7-[[3-(3,4-dimethoxyphenyl)-1,2,4-oxadiazol-5-yl]methyl]-3,7-dihydro-1,3-dimethyl-1H-purine-2,6-dione;
7-[[5-(4-bromophenyl)-1,3,4-oxadiazol-2-yl]methyl]-8-chloro-3,7-dihydro-1,3-dimethyl-1H-purine-2,6-dione;
8-chloro-3,7-dihydro-1,3-dimethyl-7-[[3-[3-(trifluoromethyl)phenyl]-1,2,4-oxadiazol-5-yl]methyl]-1H-purine-2,6-dione;
8-chloro-3,7-dihydro-1,3-dimethyl-7-[[2-[4-(1-methylethyl)phenyl]-4-thiazolyl]methyl]-1H-purine-2,6-dione;
8-chloro-7-[[5-(3-chlorophenyl)-2-oxazolyl]methyl]-3,7-dihydro-1,3-dimethyl-1H-purine-2,6-dione;
7-[[2-(5-bromo-2-methoxyphenyl)-4-thiazolyl]methyl]-8-chloro-3,7-dihydro-1,3-dimethyl-1H-purine-2,6-dione;
8-chloro-3,7-dihydro-1,3-dimethyl-7-[[3-(2-methylphenyl)-1,2,4-oxadiazol-5-yl]methyl]-1H-purine-2,6-dione;
8-chloro-3,7-dihydro-7-[[3-(3-methoxyphenyl)-1,2,4-oxadiazol-5-yl]methyl]-1,3-dimethyl-1H-purine-2,6-dione;
8-chloro-3,7-dihydro-7-[[3-(2-methoxyphenyl)-1,2,4-oxadiazol-5-yl]methyl]-1,3-dimethyl-1H-purine-2,6-dione;
8-chloro-3,7-dihydro-7-[[3-(4-methoxyphenyl)-1,2,4-oxadiazol-5-yl]methyl]-1,3-dimethyl-1H-purine-2,6-dione;
8-chloro-7-[[3-(4-chlorophenyl)-1,2,4-oxadiazol-5-yl]methyl]-3,7-dihydro-1,3-dimethyl-1H-purine-2,6-dione;
8-chloro-3,7-dihydro-1,3-dimethyl-7-[[5-(4-methylphenyl)-1,2,4-oxadiazol-3-yl]methyl]-1H-purine-2,6-dione;
8-chloro-3,7-dihydro-1,3-dimethyl-7-[[5-methyl-2-(2-thienyl)-4-oxazolyl]methyl]-1H-purine-2,6-dione;
8-chloro-7-[[5-(4-chlorophenyl)-1,3,4-oxadiazol-2-yl]methyl]-3,7-dihydro-1,3-dimethyl-1H-purine-2,6-dione;
8-chloro-7-[(1,3-diphenyl-1H-pyrazol-4-yl)methyl]-3,7-dihydro-1,3-dimethyl-1H-purine-2,6-dione;
8-chloro-7-[[2-(2,3-dimethoxyphenyl)-4-thiazolyl]methyl]-3,7-dihydro-1,3-dimethyl-1H-purine-2,6-dione;
8-chloro-7-[[5-(2-furanyl)-3-isoxazolyl]methyl]-3,7-dihydro-1,3-dimethyl-1H-purine-2,6-dione;
8-chloro-3,7-dihydro-1,3-dimethyl-7-[[5-(2-thienyl)-1,3,4-oxadiazol-2-yl]methyl]-1H-purine-2,6-dione;
8-chloro-3,7-dihydro-1,3-dimethyl-7-[[3-(2-thienyl)-1,2,4-oxadiazol-5-yl]methyl]-1H-purine-2,6-dione;
8-chloro-3,7-dihydro-1,3-dimethyl-7-[[2-(2-thienyl)-4-oxazolyl]methyl]-1H-purine-2,6-dione;
8-chloro-3,7-dihydro-1,3-dimethyl-7-[[5-(2-thienyl)-3-isoxazolyl]methyl]-1H-purine-2,6-dione;
8-chloro-7-[[5-(2-furanyl)-1,3,4-oxadiazol-2-yl]methyl]-3,7-dihydro-1,3-dimethyl-1H-purine-2,6-dione;
8-chloro-7-[[2-(4-fluorophenyl)-4-thiazolyl]methyl]-3,7-dihydro-1,3-dimethyl-1H-purine-2,6-dione;
8-chloro-7-[[2-(4-chlorophenyl)-4-thiazolyl]methyl]-3,7-dihydro-1,3-dimethyl-1H-purine-2,6-dione;
7-[[5-(2-bromophenyl)-1,3,4-oxadiazol-2-yl]methyl]-8-chloro-3,7-dihydro-1,3-dimethyl-1H-purine-2,6-dione;
8-chloro-3,7-dihydro-7-[[2-(3-methoxyphenyl)-4-thiazolyl]methyl]-1,3-dimethyl-1H-purine-2,6-dione;
8-chloro-7-[[3-(4-fluorophenyl)-1,2,4-oxadiazol-5-yl]methyl]-3,7-dihydro-1,3-dimethyl-1H-purine-2,6-dione;
8-chloro-3,7-dihydro-1,3-dimethyl-7-[[2-(4-methylphenyl)-4-thiazolyl]methyl]-1H-purine-2,6-dione;
8-chloro-3,7-dihydro-1,3-dimethyl-7-[(5-phenyl-1,3,4-oxadiazol-2-yl)methyl]-1H-purine-2,6-dione;
8-chloro-3,7-dihydro-1,3-dimethyl-7-[(2-phenyl-4-oxazolyl)methyl]-1H-purine-2,6-dione;
   and
8-chloro-3,7-dihydro-1,3-dimethyl-7-[(5-phenyl-2-oxazolyl)methyl]-1H-purine-2,6-dione.

Particular compounds of the first aspect of the invention (including all embodiments and alternatives thereof) that may be mentioned include those described in the examples provided herein, and pharmaceutically acceptable salts thereof. For example, particular compounds that may be mentioned include those described in Table 1 herein below, and pharmaceutically acceptable salts thereof.

### Medical Uses

As indicated herein, the compounds of the invention, and therefore compositions and kits comprising the same, are useful as pharmaceuticals.

Thus, according to a second aspect of the invention there is provided a compound of the first aspect of the invention, as hereinbefore defined (i.e. a compound as defined in the first aspect of the invention, including all embodiments and particular features thereof), for use as a pharmaceutical (or for use in medicine).

For the avoidance of doubt, references to compounds as defined in the first aspect of the invention will include references to compounds of formula I (including all embodiments thereof) and pharmaceutically acceptable salts thereof.

As indicated herein, the compounds of the invention may be of particular use in treating cancers.

Thus, in a third aspect of the invention, there is provided a compound of the first aspect of the invention, as hereinbefore defined, for use in the treatment of cancer.

In an alternative third aspect of the invention, there is provided a method of treating cancer comprising administering to a patient in need thereof a therapeutically effective amount of a compound of the first aspect of the invention, as hereinbefore defined.

In a further alternative third aspect of the invention, there is provided the use of a compound of the invention, as hereinbefore defined, for the manufacture of a medicament for the treatment of cancer.

The skilled person will understand that references to the treatment of a particular condition (or, similarly, to treating that condition) take their normal meanings in the field of medicine. In particular, the terms may refer to achieving a reduction in the severity of one or more clinical symptom associated with the condition. For example, in the case of a cancer, the term may refer to achieving a reduction of the amount (i.e. the number) of cancerous cells present (which may, in the case of a cancer forming a solid tumour, be indicated by a reduction in tumour volume).

As used herein, references to patients will refer to a living subject being treated, including mammalian (e.g. human) patients.

As used herein, the term effective amount will refer to an amount of a compound that confers a therapeutic effect on the treated patient. The effect may be observed in a manner that is objective (i.e. measurable by some test or marker) or subjective (i.e. the subject gives an indication of and/or feels an effect). In particular, the effect may observed (e.g. measured) in a manner that is objective.

The skilled person will understand that parameters such as number of tumour cells present and/or the volume of a tumour (and, consequently, the reduction thereof as measured between two or more time points) may be observed and measured using techniques well-known to those skilled in the art, such as by using scanning techniques (e.g. MRI scan) and/or the taking and analysis of samples (such as blood samples)

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral or parenteral administration (e.g. oral or parenteral administration). All prodrugs of the compounds of the first aspect of the invention are included within the scope of the invention.

Various prodrugs of compounds of the invention that may be formed will be known to those skilled in the art. For example, particular prodrugs that may be mentioned include esters (i.e. esters formed from carboxylic acid groups in compounds of formula I), such as C₁₋₆ alkyl, phenyl and benzyl esters (e.g. C₁₋₄ alkyl esters, such as t-butyl esters).

For the avoidance of doubt, where compounds of the invention are present in the form of prodrugs of compounds of formula I, such prodrugs may also be present in the form of pharmaceutically acceptable salts, such as those described herein.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the active compounds of the invention to which they are metabolised), may also be described as “prodrugs”.

Thus, the compounds of the invention are useful because they possess pharmacological activity, and/or are metabolised in the body following oral or parenteral administration to form compounds that possess pharmacological activity.

As described herein, the compounds of the first aspect of the invention may be useful in the treatment of cancers.

In certain embodiments (i.e. certain embodiments of the third aspect of the invention), the cancer is a solid tumour cancer, such as a cancer selected from sarcomas, carcinomas, and lymphomas.

As described herein, the compounds of the first aspect of the invention may find particular utility in the treatment of hormone-dependent cancers. Thus, in certain embodiments, the cancer is a hormone-responsive cancer, including: hormone dependent cancers of the breast, ovaries and endometrium in females; and cancers of the prostate and testicles in males.

The skilled person will understand that references to hormone-dependent cancers may refer to cancers where there is over-expression of hormone receptors. For example, in breast cancer occurring in a female (human) patient, the term may refer to a cancer with high (i.e. increased, e.g. compared to cancers that are not hormone dependent) levels of oestrogen or progesterone receptors, which may be observed and diagnosed using knowledge and techniques well-known to those skilled in the art (e.g. by tumour biopsy and analysis thereof). Similar techniques may be employed to identify increased levels of the androgen receptor in prostate cancer occurring in male (human) patients.

In particular embodiments of the third aspect of the invention, the cancer is breast cancer (i.e. a cancer of the breast tissue). More particularly, the cancer may be a breast cancer in a female patient.

For the avoidance of doubt, in particular embodiments the cancer is hormone-responsive breast cancer.

In more particular embodiments, the cancer is oestrogen-responsive (ER) or progesterone-responsive (PR) breast cancer.

The skilled person will understand that the characterisation of a cancer as being ER or PR responsive can be based on an analysis performed using techniques well-known to those skilled in the art, such as through analysis of a sample of tumour cells obtained from the patient (i.e. cells taken through biopsy).

In particular, hormone-dependent cancers may be cancers occurring in patients (e.g. human patients) of reproductive age.

More particularly, the cancer may be ER or PR breast cancer in a female patient, such as a female patient of reproductive age (i.e. a female patient being post-puberty and pre-menopause).

### Pharmaceutical Compositions

As described herein, compounds of the first aspect (and, therefore, also the second and third aspects) of the invention are useful as pharmaceuticals. Such compounds may be administered alone or may be administered by way of known pharmaceutical compositions/formulations.

In a fourth aspect of the invention, there is provided a pharmaceutical composition comprising a compound of the first aspect of the invention as defined herein, and optionally one or more pharmaceutically-acceptable excipient.

As used herein, the term pharmaceutically-acceptable excipients includes vehicles, adjuvants, carriers, diluents, pH adjusting and buffering agents, tonicity adjusting agents, stabilizers, wetting agents and the like. In particular, such excipients may include adjuvants, diluents or carriers.

The skilled person will understand that references herein to compounds of the first aspect of the invention being for particular uses (and, similarly, to uses and methods of use relating to compounds of the invention) may also apply to pharmaceutical compositions comprising compounds of the invention as described herein.

In a fifth aspect of the invention, there is provided a pharmaceutical composition as defined in the fourth aspect of the invention for use in the treatment of cancer (as defined herein with reference to the third aspect of the invention).

The skilled person will understand that compounds of the first aspect of the invention may act systemically and/or locally (i.e. at a particular site), and may therefore be administered accordingly using techniques known to those skilled in the art.

The skilled person will understand that compounds and compositions as described in the first to fifth aspects of the invention will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form. Pharmaceutical compositions as described herein will include compositions in the form of tablets, capsules or elixirs for oral administration, suppositories for rectal administration, sterile solutions or suspensions for parenteral or intramuscular administration, and the like. Alternatively, particularly where such compounds of the invention act locally, pharmaceutical compositions may be formulated for topical administration.

Thus, in particular embodiments of the fourth and fifth aspects of the invention, the pharmaceutical formulation is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The skilled person will understand that compounds of first aspect of the invention, and pharmaceutically-acceptable salts thereof, may be administered (for example, as formulations as described hereinabove) at varying doses, with suitable doses being readily determined by one of skill in the art. Oral, pulmonary and topical dosages (and subcutaneous dosages, although these dosages may be relatively lower) may range from between about 0.01 μg/kg of body weight per day (μg/kg/day) to about 200 μg/kg/day, preferably about 0.01 to about 10 μg/kg/day, and more preferably about 0.1 to about 5.0 μg/kg/day. For example, when administered orally, treatment with such compounds may comprise administration of a formulations typically containing between about 0.01 μg to about 2000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 25 mg, 50 mg, 100 mg or 200 mg twice daily).

In any event, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

### Combinations and Kits-of-Parts

The skilled person will understand that treatment with compounds of the first aspect of the invention may further comprise (i.e. be combined with) further treatment(s) for the same condition. In particular, treatment with compounds of the first aspect of the invention may be combined with means for the treatment of cancer (such as a cancer as described herein), such as treatment with one or more other therapeutic agent that is useful in the in the treatment of cancer and/or one or more physical method used in the treatment of cancer (such as treatment through surgery), as known to those skilled in the art.

As described herein, compounds of the invention may also be combined with one or more other (i.e. different, e.g. agents other than compounds of formula I) therapeutic agents that are useful in the treatment of cancer. Such combination products that provide for the administration of a compound of the invention in conjunction with one or more other therapeutic agent may be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the one or more other therapeutic agent).

Thus, according to a sixth aspect of the invention, there is provided a combination product comprising:

(I) a compound of the first aspect of the invention, as hereinbefore defined (i.e. in the first aspect of the invention, including all embodiments and particular features thereof); and
(II) one or more other therapeutic agent that is useful in the treatment of cancer (such as a cancer as described in the third aspect of the invention),
wherein each of components (I) and (II) is formulated in admixture, optionally with one or more a pharmaceutically-acceptable excipient.

In a seventh aspect of the invention, there is provided a kit-of-parts comprising:

(a) a pharmaceutical formulation as hereinbefore defined (i.e. in the fifth aspect of the invention); and
(b) one or more other therapeutic agent that is useful in the treatment of cancer (such as a cancer as described in the third aspect of the invention), optionally in admixture with one or more pharmaceutically-acceptable excipient,
which components (a) and (b) are each provided in a form that is suitable for administration in conjunction (i.e. concomitantly or sequentially) with the other.

With respect to the kits-of-parts as described herein, by “administration in conjunction with” (and similarly “administered in conjunction with”) we include that respective formulations are administered, sequentially, separately or simultaneously, as part of a medical intervention directed towards treatment of the relevant condition.

Thus, in relation to the present invention, the term “administration in conjunction with” (and similarly “administered in conjunction with”) includes that the two active ingredients (i.e. a compound of the first aspect of the invention and a further treatment for cancer, or compositions comprising the same) are administered (optionally repeatedly) either together, or sufficiently closely in time, to enable a beneficial effect for the patient, that is greater, over the course of the treatment of the relevant condition, than if either agent is administered (optionally repeatedly) alone, in the absence of the other component, over the same course of treatment. Determination of whether a combination provides a greater beneficial effect in respect of, and over the course, of treatment of a particular condition will depend upon the condition to be treated or prevented, but may be achieved routinely by the skilled person.

Further, in the context of the present invention, the term “in conjunction with” includes that one or other of the two formulations may be administered (optionally repeatedly) prior to, after, and/or at the same time as, administration of the other component. When used in this context, the terms “administered simultaneously” and “administered at the same time as” includes instances where the individual doses of the compound of the invention and the additional compound for the treatment of cancer, or pharmaceutically acceptable salts thereof, are administered within 48 hours (e.g. within 24 hours, 12 hours, 6 hours, 3 hours, 2 hours, 1 hour, 45 minutes, 30 minutes, 20 minutes or 10 minutes) of each other.

Other therapeutic agents useful in the treatment of cancer (such as those cancers described in the third aspect of the invention) will be well-known to those skilled in the art. For example, such other therapeutic agents may include:

PARP (poly ADP ribose polymerase) inhibitors, such as iniparib, talazoparib, veliparib, olaparib, and rucaparib;
PARG (poly ADP ribose glycohydrolase) inhibitors;
aromatase inhibitors, such as anastrazole, letrozole, exemestane, vorozole, formestane, and fadrozole;
hormone receptor antagonists, such as tamoxifen, clomefine, ormeloxifene, raloxifene, toremifene, lasofoxifene, ospemifene, and fulvestrant.

### Preparation of Compounds/Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the first aspect of the invention, as hereinbefore defined, with one or more pharmaceutically-acceptable excipient.

In further aspects of the invention, there is provided a process for the preparation of a combination product or kit-of-parts as hereinbefore defined, which process comprises bringing into association a compound of the first aspect of the invention, as hereinbefore defined, with the other therapeutic agent that is useful in the treatment of cancer, and at least one pharmaceutically-acceptable excipient.

As used herein, references to bringing into association will mean that the two components are rendered suitable for administration in conjunction with each other.

Thus, in relation to the process for the preparation of a kit-of-parts as hereinbefore defined, by bringing the two components “into association with” each other, we include that the two components of the kit-of-parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the first aspect of the invention as described herein may be prepared in accordance with techniques that are well known to those skilled in the art, such as those described in the examples provided hereinafter.

According to an eighth aspect of the invention there is provided a process for the preparation of a compound of the first aspect of the invention as hereinbefore defined, comprising the step of:

(i) reacting a compound of formula II

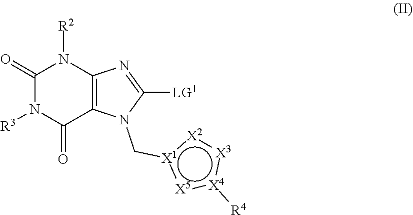

wherein R² to R⁴ and X¹ to X⁵ are as defined herein (i.e. in the first aspect of the invention, as provided in the definition of compounds of formula I) and LG¹ represents a suitable leaving group (such as halo, e.g. bromo), with a compound of formula III

H—R¹ (III)

wherein R¹ is as defined herein, in the presence of a suitable solvent (such as a polar protic solvent; for example, methanol, ethanol or iso-propanol, e.g. ethanol, or a polar aprotic solvent; for example, THF, DMF or DMSO), under conditions known to those skilled in the art;
(ii) reacting a compound of formula IV

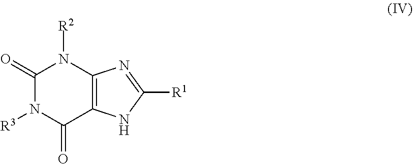

wherein R¹ to R³ are as defined herein, with a compound of formula V

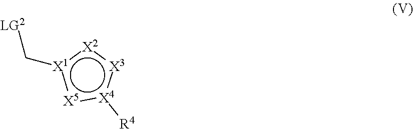

wherein X¹ to X⁵ and R⁴ are as defined herein, and LG² represents a suitable leaving group (such as halo (e.g. Cl or Br), in the presence of a suitable solvent (such as a polar aprotic solvent; for example THF, DMF or DMSO, e.g. DMF) and a suitable base (such as a alkali metal carbonate; for example, Li₂CO₃, Na₂CO₃ or K₂CO₃, e.g. K₂CO₃), under conditions known to those skilled in the art; or
(iii) reaction of a protected derivative of a compound of formula I (i.e. a compound of formula I as defined herein but further substituted with a suitable protecting group as known to those skilled in the art, such as those described herein), under conditions suitable for the removal of the protecting group(s), as known to those skilled in the art.

Compounds of formulae II, III, IV and V are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

The skilled person will understand that compounds of formula may be novel. As such, the present invention may also include compounds of formula II, as defined herein.

Particular compounds of formula that may be mentioned include those in which LG represents halo.

In particular, compounds of formula may be prepared by reaction of a compound of formula IIa

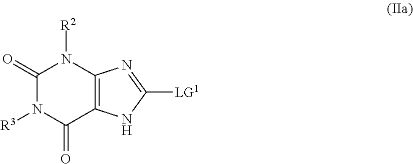

wherein R², R³ and LG¹ are as defined herein, with a compound of formula V as defined herein, in the presence of a suitable solvent (such as a polar aprotic solvent; for example, THF, DMF or DMSO, e.g. DMF) and a suitable base (such as an alkali metal carbonate; for example, Li₂CO₃, Na₂CO₃ or K₂CO₃, e.g. K₂CO₃), under conditions known to those skilled in the art.

Compounds of formula IV, particularly wherein R¹ represents alkyl (or optionally substituted alkyl) as defined herein, may be prepared by the reaction of a compound of formula VI,

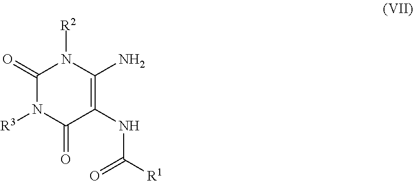

wherein R¹, R² and R³ are as defined herein, in the presence of a suitable base (for example, an alkali metal hydroxide, e.g. sodium hydroxide), under conditions known to those skilled in the art (for example, at elevated temperature, e.g. around 100° C., and in a suitable solvent, such as a polar protic solvent, e.g. MeOH).

Compounds of formula V may be prepared by reaction of a compound of formula VII,

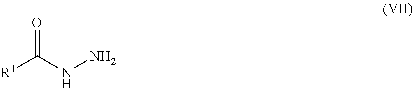

wherein R¹ is as defined herein, with a compound of formula VIII,

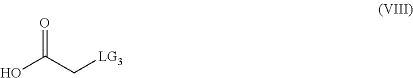

wherein LG³ represents a suitable leaving group (such as halo, e.g. Cl), in the presence of a suitable chlorinating agent (for example, POCl₃, SOCl₂ or oxalyl chloride, e.g. POCl₃), under conditions known those skilled in the art (such as at elevated temperature, e.g. at about 100-150° C.).

Similarly, compounds of formulae IIa, VI, VII and VIII are either commercially available, are known in the literature, or may be obtained either by analogy with the processes described herein, or by conventional synthetic procedures, in accordance with standard techniques, from available starting materials using appropriate reagents and reaction conditions.

The skilled person will understand that the substituents as defined herein (e.g. R¹ to R⁴), and substituents thereon, may be modified one or more times, after or during the processes described above for the preparation of compounds of the first aspect of the invention by way of methods that are well known to those skilled in the art. Examples of such methods include substitutions, reductions, oxidations, dehydrogenations, alkylations, dealkylations, acylations, hydrolyses, esterifications, etherifications, halogenations and nitrations. The precursor groups can be changed to a different such group, or to the groups defined in formula I, at any time during the reaction sequence. The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Compounds of the first aspect of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula I).

It will be appreciated by those skilled in the art that, in the processes described above and hereinafter, the functional groups of intermediate compounds may need to be protected by protecting groups. The protection and deprotection of functional groups may take place before or after a reaction in the above-mentioned schemes.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999), the contents of which are incorporated herein by reference.

Without wishing to be bound by theory, it is believed that the ability of compounds as described herein to act as potent inhibitors of the enzyme NUDT5 renders them particularly effective in the treatment of cancers, such as ER and PR breast cancer.

Compounds of the first aspect of the invention may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

### BRIEF DESCRIPTION OF THE FIGURES

FIG. 1 shows NUDT5 stabilization by inhibitors when added to cell lysates, as described in Biological Assay 2.

FIG. 2 shows NUDT5 stabilization by inhibitors added to cells in culture, as described in Biological Assay 2.

FIG. 3 shows the isothermal dose response fingerprint for certain inhibitors, as described in Biological Assay 2.

FIG. 4(a) shows thin layer chromatography (TLC) of products formed following processing of ³²P-PAR by recombinant PARG and NUDT5 in the absence or presence of Compound 19 and PPᵢ in vitro, as described in Biological Assay 3.

FIG. 4(b) shows the concentration of inhibitors used in T47D^{WT/M} cell culture experiments, as well as the chemical structures and IC₅₀ values, as described in Biological Assay 3.

FIG. 4(c) shows serum-starved T47D^{WT} cells expressing Nuc-luc FRTTO luciferase construct in the absence or presence of NUDT5 inhibitors prior to treatment with 10 nM R5020 and luminescence measurement, as described in Biological Assay 3. Top, a representative measurement of bioluminescence intensity over 60 minutes of R5020 treatment; bottom, box-and-whisker plots of bioluminescence quantitations from six independent experiments (center line, median; box limits, upper and lower quartiles; whiskers, minima and maxima; individual data points also shown). ns—not significant, *—p<0.05, **—p<0.01, ***—p<0.001; repeated measures two-way ANOVA analysis.

FIG. 4(d) shows histone displacement determined by chromatin immunoprecipitation (ChIP) using a histone H1-specific antibody prior to (Starved) or following 30 minutes of R5020 (+R5020) in the presence or absence of NUDT5 inhibitors in T47D^{M} cells, as described in Biological Assay 3. Data from a representative experiment is presented as mean fold change (+R5020/Starved)±SD of five different histone H1 contact regions.

FIG. 4(e) shows progesterone-dependent and -independent gene expression analysis in the presence or absence (−) of NUDT5 inhibitors following R5020 treatment (6 hours) by RT-qPCR with T47D^{M} cells, as described in Biological Assay 3. Data represents the mean±SEM log(mRNA abundance) normalized to serum-starved cells without R5020 treatment from three (progesterone-dependent) or two (progesterone-independent) independent experiments. ns—not significant, *—p<0.05, **—p<0.01; one-way ANOVA analysis.

FIG. 4(f) shows R5020-induced cell proliferation of T47D^{M} cells in the absence or presence of NUDT5 inhibitors was assayed by BrdU incorporation after 24 hours, as described in Biological Assay 3. Mean±SEM of individual BrdU chemiluminescence signals are displayed without R5020 treatment (Starved) and following R5020 treatment (+R5020) from two independent experiments. RLU; relative luminescence units. ns—not significant, **—p<0.01, ****—p<0.0001; one-way ANOVA analysis.

FIG. 5 shows the results of CETSA experiments performed with cells treated in culture, as described in Biological Assay 2. Means±SEM from two independent experiments are shown. NUDT5 stabilization was relative to the 37° C. DMSO control and SOD1 was used as a loading control. ns—not significant; *—p<0.05; one-way ANOVA.

For the avoidance of doubt, the numbering used in figure legends refers to the numbering of compounds of the examples as provided herein.

### EXAMPLES

The present invention will be further described by reference to the following examples, which are not intended to limit the scope of the invention in any way.

In the event that there is a discrepancy between nomenclature and any compounds depicted graphically, then it is the latter that presides (unless contradicted by any experimental details that may be given or unless it is clear from the context).

### Experimental Procedures

Starting materials and intermediates used in the synthesis of compounds described herein are commercially available or can be prepared by the methods described herein or by methods known in the art.

Experiments were generally carried out under inert atmosphere (nitrogen or argon), particularly in cases where oxygen- or moisture-sensitive reagents or intermediates were used.

Mass spectrometry data are reported from liquid chromatography-mass spectrometry (LC-MS) using electrospray ionization. Chemical shifts for NMR data are expressed in parts per million (ppm, δ) referenced to residual peaks from the deuterated solvent used.

For syntheses referencing general procedures, reaction conditions (such as length of reaction or temperature) may vary. In general, reactions were followed by thin layer chromatography or LC-MS, and subjected to work-up when appropriate. Purifications may vary between experiments: in general, solvents and the solvent ratios used for eluents/gradients were chosen to provide an appropriate R_{f} and/or retention time.

Where applicable, compound names indicated in respect of the following examples have been generated using the structure naming function of ChemBioDraw Ultra, Version 12.0.

### General Procedures

### General Procedure A: Synthesis of Substituted 2-chloromethyl oxadiazoles

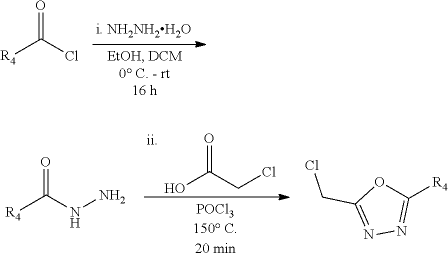

i) The corresponding acid chloride (1 equiv.) was dissolved in dichloromethane (0.2 mol/L) and then added dropwise to a stirring solution of hydrazine hydrate (4 equiv.) in EtOH (0.8 mol/L) at 0° C. The reaction was allowed to warm to room temperature and monitored for completion using TLC. Upon completion, the dichloromethane was removed under reduced pressure. The resultant suspension was then cooled to 0° C. and the precipitated product was collected by suction filtration and washed with cold water. This crude product was dried overnight in vacuo and used without further purification.
ii) Benzohydrazide (1 equiv.) was added to 2-chloroacetic acid (1 equiv.) dissolved in POCl₃ (0.1-0.5 mol/L). The vessel was thoroughly flushed with nitrogen gas and sealed, then the mixture was heated to 150° C. for 15 minutes in a Biotage microwave reactor. Solids were then rinsed down into the solution with a small amount of POCl₃ and the reaction was heated at 150° C. for an additional 5 minutes. Upon completion, POCl₃ was removed under reduced pressure. The crude mixture was purified using silica gel chromatography in a mixture of iso-hexane and ethyl acetate.

### General Procedure B: Alkylation of 8-Functionalized theophyllines

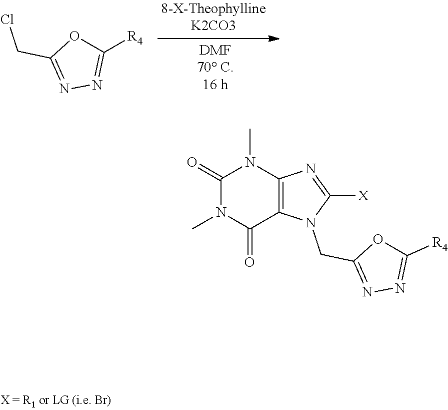

The corresponding 2-chloromethyl-oxadizole was added to a suspension of 8-functionalized theophylline (1.1 equiv.) and K₂CO₃ (1.2 equiv.) in DMF (˜0.2 mol/L) and stirred at 70° C. overnight in a sealed vessel. Upon completion, the reaction mixture was diluted with H₂O and organics were extracted with EtOAc. The combined organics were washed with 1 M HCl, saturated NaHCO₃, H₂O and brine, followed by additional drying over MgSO₄. The crude mixture was then purified by automated flash chromatography in a mixture of iso-hexane and ethyl acetate.

### General Procedure B′: Alkylation of 8-bromo- or 8-chloro-theophylline

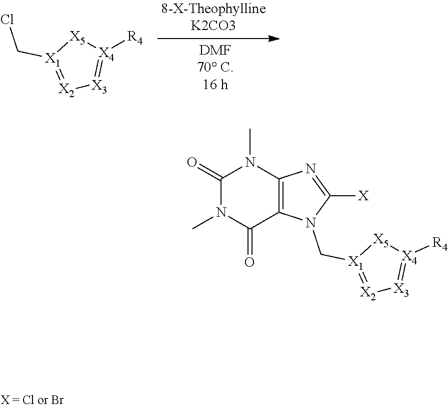

The corresponding R₄ substituted, 2-chloromethyl-heterocycles were added to a suspension of 8-bromo- or 8-chloro-theophylline (1.1 equiv.) and K₂CO₃ (1.2 equiv.) in DMF (˜0.2 mol/L) and stirred at 70° C. overnight in a sealed vessel. Upon completion, the reaction mixture was diluted with H₂O and organics were extracted with EtOAc. The combined organics were washed with 1 M HCl, saturated NaHCO₃, H₂O and brine, followed by additional drying over MgSO₄. The crude mixture was then purified by automated flash chromatography in a mixture of iso-hexane and ethyl acetate.

### General Procedure C: Aromatic Substitution of 8-bromotheophylline

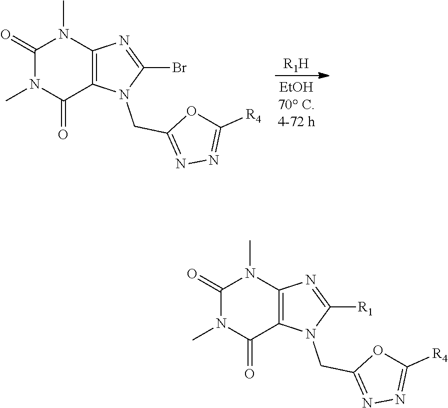

Amines or thiols (2-5 equivalents) were added to functionalized 8-Br-theophylline suspended in ethanol, heated to 70° C. and stirred overnight. Upon completion, the reaction mixture was cooled to 0° C. and the precipitated product was collected by suction filtration. In cases where the product did not precipitate or was of insufficient purity, the crude mixture was purified using automated flash chromatography in mixtures of iso-hexane and ethyl acetate or dichloromethane and methanol.

### General Procedure C′: Aromatic Substitution of 8-bromo- or 8-chloro-theophylline

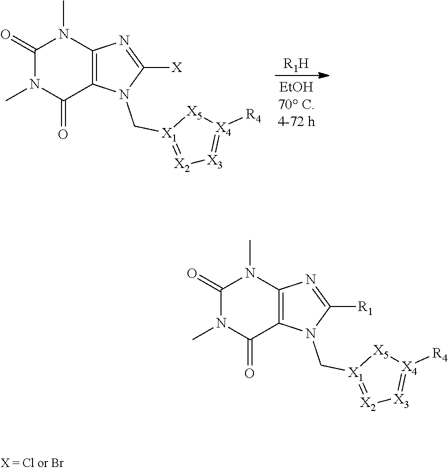

Amines or thiols (2-5 equivalents) were added to functionalized 8-Br- or 8-Cl-theophylline suspended in ethanol, heated to 70° C. and stirred overnight. Upon completion, the reaction mixture was cooled to 0° C. and the precipitated product was collected by suction filtration. In cases where the product did not precipitate or was of insufficient purity, the crude mixture was purified using preparative HPLC, crystallization, or automated flash chromatography in mixtures of iso-hexane and ethyl acetate or dichloromethane and methanol.

### General Procedure D: Synthesis of 8-alkyl-theophylline Derivatives

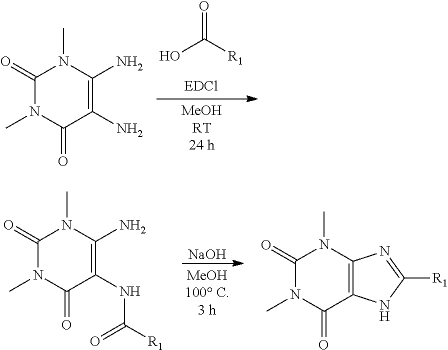

8-alkyl theophylline derivatives were prepared as previously reported (Merlos, M., et al. Eur. J. Med. Chem. 25, 653-658 (1990)). Briefly, 5,6-diamino-1,3-dimethyl-1,2,3,4-tetrahydropyrimidine-2,4-dione was dissolved in methanol followed by the addition of the desired carboxylic acid and 1-ethyl-3-(3-dimethylaminopropyl)carbodiimide (EDCl). Upon disappearance of the starting material (˜24 hours), methanol was evaporated and the crude mixture was purified by silica gel chromatography. The resultant carboxamide was then dissolved in a mixture of MeOH and 10% NaOH in H₂O w/w (1:2) and stirred at 100° C. for 3 hours. Upon completion, the mixture was cooled to room temperature, acidified with HCl and the precipitated product was collected by suction filtration and washed with water.

### General Procedure D′: t-butoxycarbonyl (Boc) or 2,4-dimethoxybenzyl (Dmb) Deprotection of R₁ Derivatives

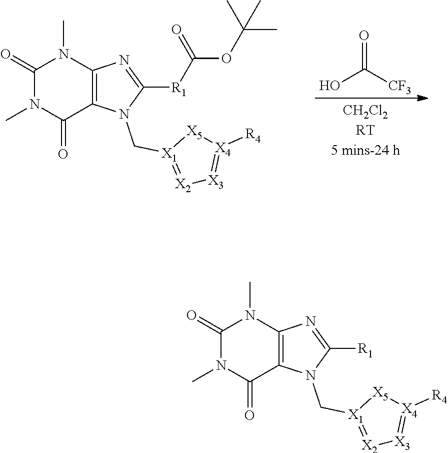

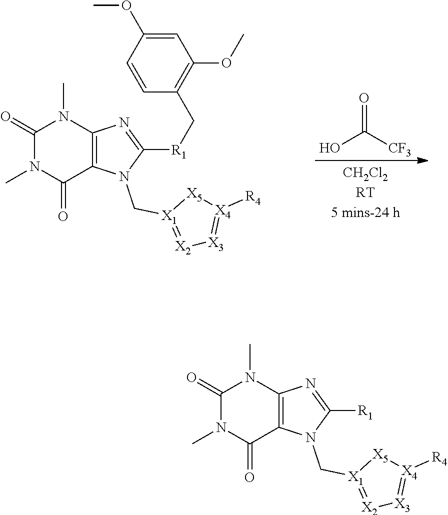

In some instances, a t-butoxycarbonyl (Boc) or a 2,4-dimethoxybenzyl (DMB) protecting group was present on the nucleophilic R₁ groups used in General Procedure C′. In these cases, the subsequent deprotection to give the unprotected R₁ was carried out under standard acidic Boc/DMB-deprotection conditions. Briefly, trifluoroacetic acid (TFA) was added to functionalized Boc-protected intermediates dissolved in dichloromethane (CH₂Cl₂). Reaction progress was monitored by thin layer chromatography or LC-MS. Upon completion, CH₂Cl₂ and TFA were removed under reduced pressure and the resultant residue was either used without subsequent purification, or could be purified by flash column chromatography, preparative HPLC or crystallization.

### General Chemical Syntheses

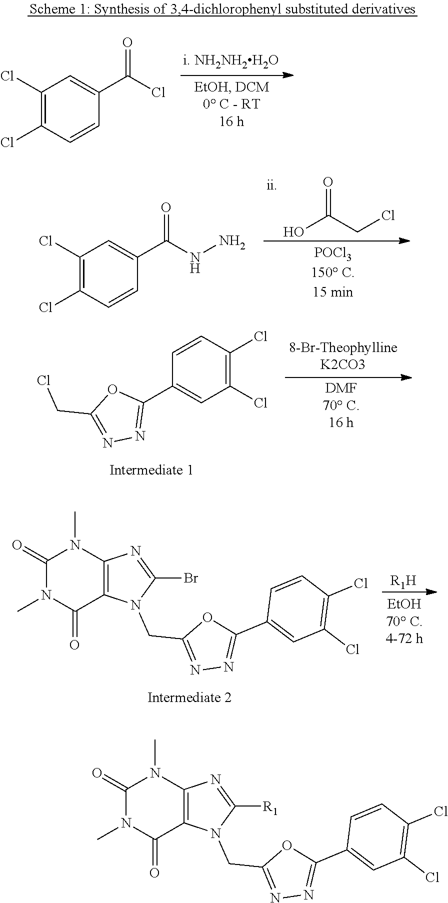

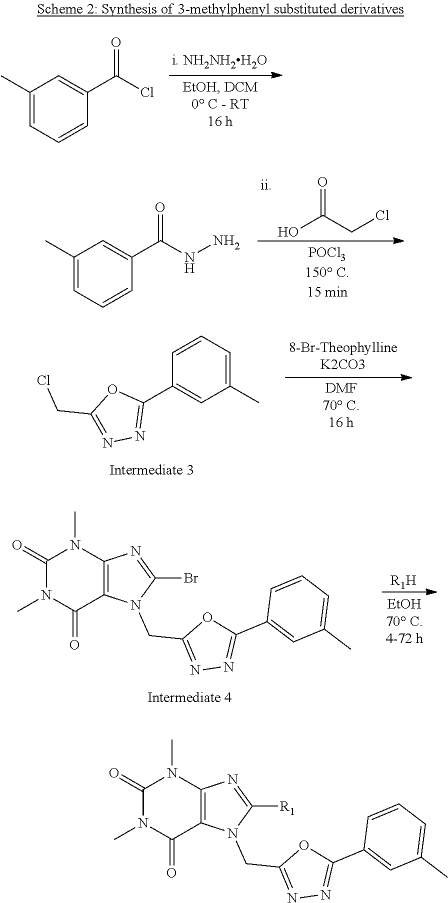

### Synthesis of Intermediates

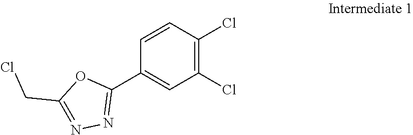 [SMILES: ClCc1nnc(-c2ccc(Cl)c(Cl)c2)o1]

2-(chloromethyl)-5-(3,4-dichlorophenyl)-1,3,4-oxadiazole was prepared according to General Procedure A on a 5 mmol scale. ¹H NMR (400 MHz, CHLOROFORM-d) δ ppm 4.79 (s, 2H), 7.63 (d, J=8.37 Hz, 1H), 7.93 (dd, J=8.37, 2.05 Hz, 1H), 8.18 (d, J=2.05 Hz, 1H). ¹³C NMR (101 MHz, CHLOROFORM-d) ppm 32.9, 123.1, 126.1, 128.8, 131.4, 133.9, 136.8, 162.6, 164.2. HPLC-MS t_{R}=1.94 minutes, 100% purity, m/z calculated for [C₉H₅Cl₃N₂O+H]=263, found 263 (713 mg, 55%).

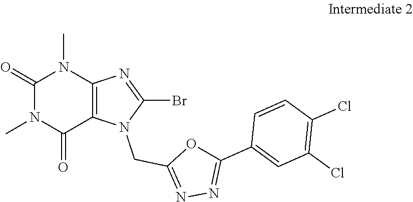 [SMILES: Cn1c(=O)c2c(nc(Br)n2Cc2nnc(-c3ccc(Cl)c(Cl)c3)o2)n(C)c1=O]

8-bromo-7-{[5-(3,4-dichlorophenyl)-1,3,4-oxadiazol-2-yl]methyl}-1,3-dimethyl-2,3,6,7-tetrahydro-1H-purine-2,6-dione was prepared according to General Procedure B on a 5 mmol scale. ¹H NMR (400 MHz, CHLOROFORM-d) ppm 3.41 (s, 3H), 3.60 (s, 3H), 5.91 (s, 2H), 7.61 (d, J=8.37 Hz, 1H), 7.86 (dd, J=8.37, 2.05 Hz, 1H), 8.11 (d, J=1.90 Hz, 1H). ¹³C NMR (101 MHz, CHLOROFORM-d) δ ppm 28.1, 30.0, 41.3, 108.8, 122.9, 126.1, 128.3, 128.8, 131.3, 133.8, 136.9, 148.4, 151.1, 154.3, 160.5, 164.1. HPLC-MS t_{R}=1.84 minutes, 100% purity, m/z calculated for [C₁₆H₁₁BrCl₂N₆O₃+H]=485.0, found 485.0, (1.03 g, 44% yield).

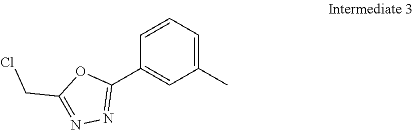

2-(chloromethyl)-5-(3-methylphenyl)-1,3,4-oxadiazole was prepared as outlined in general procedure A on a 10 mmol scale. ¹H NMR (400 MHz, CHLOROFORM-d) δ ppm 2.45 (s, 3H), 4.79 (s, 2H), 7.30-7.47 (m, 2H), 7.82-7.94 (m, 2H). HPLC-MS t_{R}=1.80 minutes, 90% purity, m/z calculated for [C₁₀H₉ClN₂O+H]=209, found 209 (1.72 g, 83%).

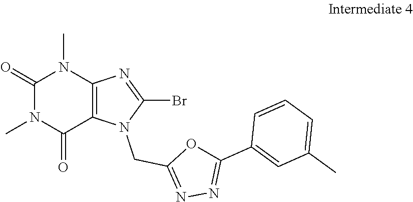 [SMILES: Cc1cccc(-c2nnc(Cn3c(Br)nc4c3c(=O)n(C)c(=O)n4C)o2)c1]

8-bromo-1,3-dimethyl-7-{[5-(3-methylphenyl)-1,3,4-oxadiazol-2-yl]methyl}-2,3,6,7-tetrahydro-1H-purine-2,6-dione was prepared as outlined in general procedure B on a 0.9 mmol scale. ¹H NMR (400 MHz, CHLOROFORM-d) ppm 2.43 (s, 3H), 3.41 (s, 3H), 3.59 (s, 3H), 5.90 (s, 2H), 7.32-7.44 (m, 2H), 7.73-7.82 (m, 1H), 7.84 (dt, J=1.50, 0.83 Hz, 1H). ¹³C NMR (101 MHz, CHLOROFORM-d) ppm 21.3, 28.1, 30.0, 41.4, 108.9, 123.0, 124.2, 127.6, 128.3, 129.0, 133.0, 139.0, 148.3, 151.2, 154.2, 159.9, 166.1. HPLC-MS t_{R}=1.70 minutes, 95.9% purity, m/z calculated for [C₁₇H₁₅BrN₆O₃+H]=431.1, found 431.1 (199 mg, 55%).

### Example Compounds

Compounds of the examples are described in Table 1 below. These compounds were prepared in accordance with the general procedures indicated.

TABLE 1

| | | Structure |
|---|---|---|
| | | Name |
| | General | ¹H-NMR, ¹³C-NMR |
| Example | Procedure | MS [M + H]⁺ |
| 1 | C⁽¹⁾ | 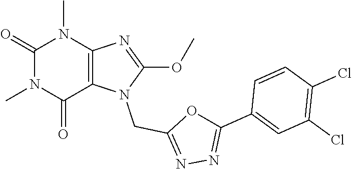 [SMILES: COc1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazol-2-yl]methyl}-8- |
| | | methoxy-1,3-dimethyl-2,3,6,7-tetrahydro-1H-purine-2,6- |
| | | dione |
| | | ¹H NMR (400 MHz, DMSO-d⁶) δ ppm 3.18 (s, 3 H), 3.41 (s, |
| | | 3 H, 4.14 (s, 3 H), 5.64 (s, 2 H), 7.85-7.95 (m, 2 H), 8.13 |
| | | (s, 1 H). ¹³C NMR (101 MHz, CHLOROFORM-d) δ ppm 27.8, |
| | | 29.9, 37.8, 58.4, 102.7, 123.1, 126.0, 128.7, 131.3, 133.8, |
| | | 136.6, 146.7, 151.6, 154.7, 156.3, 161.6, 163.9. |
| | | 437 |
| 2 | C | 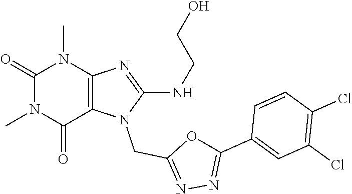 [SMILES: Cn1c(=O)c2c(nc(NCCO)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-8- |
| | | [(2-hydroxyethyl)amino]-1,3-dimethyl-2,3,6,7-tetrahydro-1H- |
| | | purine-2,6-dione |
| | | ¹H NMR (400 MHz, DMSO-d⁶) δ ppm 3.14 (s, 3 H), 3.38 (s, |
| | | 3 H), 3.43 (q, J = 6.1 Hz, 2 H), 3.56 (q, J = 5.7 Hz, 2 H), 4.77 |
| | | (t, J = 6.0 Hz, 1 H), 5.68 (s, 2 H), 7.44 (t, J = 5.5 Hz, 1 H), |
| | | 7.88-7.96 (m, 2 H), 8.18 (d, J = 1.7 Hz, 1 H). ¹³C NMR (101 |
| | | MHz, CHLOROFORM-d) δ ppm 27.8, 29.9, 37.3, 46.1, 62.3, |
| | | 102.2, 122.6, 126.1, 128.8, 131.4, 133.9, 137.1, 148.2, |
| | | 151.5, 154.1, 154.3, 161.7, 164.3. |
| | | 466 |
| 3 | C | 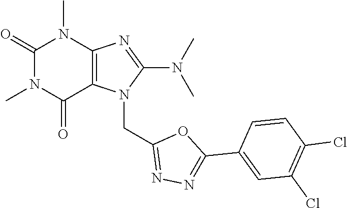 [SMILES: CN(C)c1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazol-2-yl]methyl}-8- |
| | | (dimethylamino)-1,3-dimethyl-2,3,6,7-tetrahydro-1H-purine- |
| | | 2,6-dione |
| | | ¹H NMR (400 MHz, CHLOROFORM-d) δ ppm 3.04 (s, 6 H), |
| | | 3.37 (s, 3 H), 3.56 (s, 3 H), 5.71 (s, 2 H), 7.59 (d, J = 8.34 Hz, |
| | | 1 H), 7.84 (dd, J = 8.34, 2.02 Hz, 1 H), 8.10 (d, J = 2.02 Hz, 1 |
| | | H). ¹³C NMR (101 MHz, CHLOROFORM-d) δ ppm 27.8, |
| | | 29.8, 40.4, 41.9, 104.1, 123.1, 126.0, 128.8, 131.3, 133.8, |
| | | 136.7, 148.3, 151.7, 154.6, 157.9, 162.2, 163.9. |
| | | 450 |
| 4 | C | 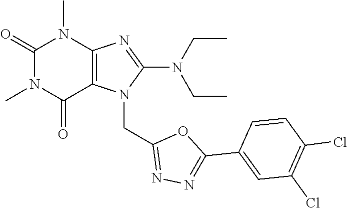 [SMILES: CCN(CC)c1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazol-2-yl]methyl}-8- |
| | | (diethylamino)-1,3-dimethyl-2,3,6,7-tetrahydro-1H-purine- |
| | | 2,6-dione |
| | | ¹H NMR (400 MHz, CHLOROFORM-d) δ ppm 2.36 (s, 3 H), |
| | | 2.57 (br. s., 4 H), 3.37 (s, 6 H), 3.57 (s, 3 H), 5.64 (s, 2 H), |
| | | 7.60 (d, J = 8.34 Hz, 1 H), 7.79-7.93 (m, 1 H), 8.11 (d, J = 1.77 |
| | | Hz, 1 H). ¹³C NMR (101 MHz, CHLOROFORM-d) δ ppm |
| | | 13.0, 27.7, 29.8, 40.1, 46.3, 104.2, 123.1, 126.0, 128.7, |
| | | 131.3, 133.7, 136.6, 148.3, 151.7, 154.6, 156.7, 162.2, |
| | | 163.7. |
| | | 478 |
| 5 | C | 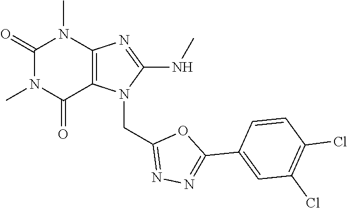 [SMILES: CNc1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazo-2-yl]methyl}-1,3- |
| | | dimethyl-8-(methylamino)-2,3,6,7-tetrahydro-1H-purine-2,6- |
| | | dione |
| | | ¹H NMR (400 MHz, DMSO-d⁶) δ ppm 2.92 (d, J = 4.58 Hz, 3 |
| | | H), 3.14 (s, 3 H), 3.40 (s, 3 H), 5.64 (s, 2 H), 7.36 (q, J = 4.53 |
| | | Hz, 1 H), 7.86-7.96 (m, 2 H), 8.10-8.19 (m, 1 H). |
| | | 436 |
| 6 | C | 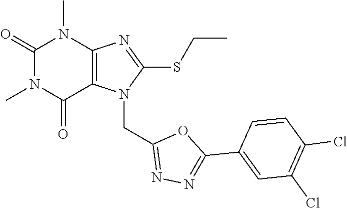 [SMILES: CCSc1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazol-2-yl]methyl}-8- |
| | | (ethylsulfanyl)-1,3-dimethyl-2,3,6,7-tetrahydro-1H-purine- |
| | | 2,6-dione |
| | | ¹H NMR (400 MHz, CHLOROFORM-d) δ ppm 1.43 (t, J = 7.35 |
| | | Hz, 3 H), 3.32 (q, J = 7.42 Hz, 2 H), 3.40 (s, 3 H), 3.60 (s, 3 |
| | | H), 5.83 (s, 2 H), 7.59 (d, J = 8.53 Hz, 1 H), 7.86 (dd, J = 8.45, |
| | | 1.97 Hz, 1 H), 8.11 (d, J = 1.90 Hz, 1 H). ¹³C NMR (101 MHz, |
| | | CHLOROFORM-d) δ ppm 14.9, 27.9, 28.0, 29.9, 39.9, |
| | | 107.8, 123.0, 126.1, 128.8, 131.3, 133.8, 136.7, 140.8, |
| | | 148.9, 151.4, 154.4, 161.1, 164.0. |
| | | 467 |
| 7 | C | 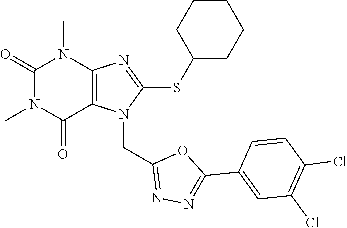 [SMILES: Cn1c(=O)c2c(nc(SC3CCCCC3)n2Cc2nnc(-c3ccc(Cl)c(Cl)c3)o2)n(C)c1=O] |
| | | 8-(cyclohexylsulfanyl)-7-{[5-(3,4-dichlorophenyl)-1,3,4- |
| | | oxadiazol-2-yl]methyl}-1,3-dimethyl-2,3,6,7-tetrahydro-1H- |
| | | purine-2,6-dione |
| | | ¹H NMR (400 MHz, CHLOROFORM-d) δ ppm 1.27-1.35 |
| | | (m, 1 H), 1.37-1.64 (m, 5 H), 1.77 (dt, J = 13.11, 3.95 Hz, 2 |
| | | H), 2.01-2.16 (m, 2 H), 3.39 (s, 3 H), 3.61 (s, 3 H), 3.76- |
| | | 3.92 (m, 1 H), 5.85 (s, 2 H), 7.53-7.65 (m, 1 H), 7.86 (dd, |
| | | J = 8.45, 1.97 Hz, 1 H), 8.10 (d, J = 1.90 Hz, 1 H). ¹³C NMR |
| | | (101 MHz, CHLOROFORM-d) δ ppm 25.4, 25.8, 28.0, 29.9, |
| | | 33.4, 40.1, 48.3, 107.7, 123.1, 126.1, 128.8, 131.3, 133.8, |
| | | 136.7, 148.9, 151.5, 151.5, 154.5, 161.3, 163.9. |
| | | 521 |
| 8 | C | 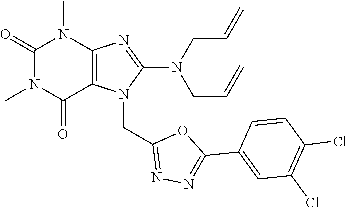 [SMILES: C=CCN(CC=C)c1nc2c(c(=O)n(C)c(=O)n2C)n1Cc1nnc(-c2ccc(Cl)c(Cl)c2)o1] |
| | | 8-[bis(prop-2-en-1-yl)amino]-7-{[5-(3,4-dichlorophenyl)- |
| | | 1,3,4-oxadiazol-2-yl]methyl}-1,3-dimethyl-2,3,6,7- |
| | | tetrahydro-1H-purine-2,6-dione |
| | | ¹H NMR (400 MHz, CHLOROFORM-d) δ ppm 3.35 (3 H, s), |
| | | 3.55 (s, 3 H), 3.94 (dt, J = 5.69, 1.26 Hz, 4 H), 5.17-5.29 (m, |
| | | 4 H), 5.66 (s, 2 H), 5.80-5.94 (m, 2 H), 7.58 (d, J = 8.53 Hz |
| | | 1 H), 7.81-7.89 (m, 1 H), 8.09 (d, J = 1.90 Hz, 1 H) |
| | | 502 |
| 9 | C | 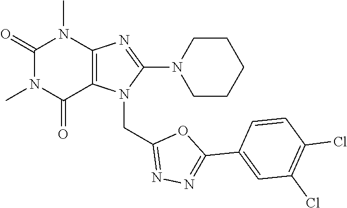 [SMILES: Cn1c(=O)c2c(nc(N3CCCCC3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-1,3- |
| | | dimethyl-8-(piperidin-1-yl)-2,3,6,7-tetrahydro-1H-purine-2,6- |
| | | dione |
| | | ¹H NMR (400 MHz, CHLOROFORM-d) δ ppm 1.58-1.71 |
| | | (m, 6 H), 3.20-3.29 (m, 4 H), 3.37 (s, 3 H), 3.61 (s, 3 H), |
| | | 5.63 (s, 2 H), 7.60 (d, J = 8.37 Hz, 1 H), 7.86 (dd, J = 8.45, 1.97 |
| | | Hz, 1 H), 8.11 (d, J = 2.05 Hz, 1 H). ¹³C NMR (101 MHz, |
| | | CHLOROFORM-d) δ ppm 23.8, 25.4, 27.8, 29.8, 40.3, 51.6, |
| | | 104.5, 123.1, 126.0, 128.7, 131.3, 133.8, 136.6, 148.1, |
| | | 151.7, 154.8, 157.5, 162.2, 163.8. |
| | | 490 |
| 10 | C | 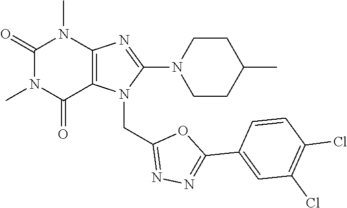 [SMILES: CC1CCN(c2nc3c(c(=O)n(C)c(=O)n3C)n2Cc2nnc(-c3ccc(Cl)c(Cl)c3)o2)CC1] |
| | | 7-{[5-(3,4-dichlorophenyl)-1,3,4-oxadiazol-2-yl]methyl}-1,3- |
| | | dimethyl-8-(4-methylpiperidin-1-yl)-2,3,6,7-tetrahydro-1H- |
| | | purine-2,6-dione |
| | | ¹H NMR (400 MHz, CHLOROFORM-d) δ ppm 0.98 (d, |
| | | J = 6.63 Hz, 3 H), 1.30 (qd, J = 12.32, 3.95 Hz, 2 H), 1.57 (ddd, |
| | | J = 11.06, 6.95, 3.79 Hz, 1 H), 1.73 (dd, J = 12.72, 2.29 Hz, 2 |
| | | H), 3.02 (td, J = 12.48, 2.53 Hz, 2 H), 3.37 (s, 3 H), 3.51 (d, |
| | | J = 12.48 Hz, 2 H), 3.56-3.62 (m, 3 H), 5.63 (s, 2 H), 7.60 (d, |
| | | J = 8.37 Hz, 1 H), 7.86 (dd, J = 8.37, 2.05 Hz, 1 H), 8.11 (d, |
| | | J = 2.05 Hz, 1 H). ¹³C NMR (101 MHz, CHLOROFORM-d) δ |
| | | ppm 21.7, 27.8, 29.8, 30.4, 33.6, 40.3, 51.0, 104.5, 123.1, |
| | | 126.0, 128.7, 131.3, 133.8, 136.6, 148.1, 151.7, 154.8, |
| | | 157.5, 162.2, 163.8 |
| | | 504 |
| 11 | C | 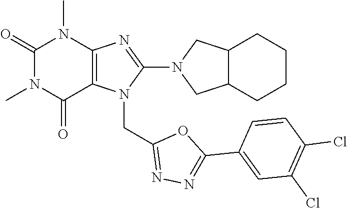 [SMILES: Cn1c(=O)c2c(nc(N3CC4CCCCC4C3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-1,3- |
| | | dimethyl-8-(octahydro-1H-isoindol-2-yl)-2,3,6,7-tetrahydro- |
| | | 1H-purine-2,6-dione |
| | | ¹H NMR (400 MHz, DMSO-d⁶) δ ppm 1.29-1.59 (m, 8 |
| | | H), 2.17-2.30 (m, 2 H), 3.15 (s, 3 H), 3.36 (s, 3 H), 3.52- |
| | | 3.55 (m, 2 H), 3.62-3.66 (m, 2 H), 5.86 (s, 2 H), 7.85-7.94 |
| | | (m, 2 H), 8.06-8.15 (m, 1 H). ¹³C NMR (101 MHz, |
| | | CHLOROFORM-d) δ ppm 22.6, 25.6, 27.7, 29.8, 37.3, 39.7, |
| | | 53.8, 102.9, 123.0, 126.0, 128.8, 131.3, 133.8, 136.7, 149.3, |
| | | 151.8, 154.2, 155.4, 162.5, 163.9 |
| | | 530 |
| 12 | C | 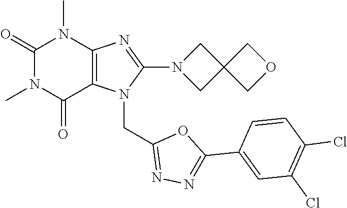 [SMILES: Cn1c(=O)c2c(nc(N3CC4(COC4)C3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-1,3- |
| | | dimethyl-8-{2-oxa-6-azaspiro[3.3]heptan-6-yl}-2,3,6,7- |
| | | tetrahydro-1H-purine-2,6-dione |
| | | ¹H NMR (400 MHz, DMSO-d⁶) δ ppm 3.15 (s, 3 H), 3.37 (s, |
| | | 3H), 4.42 (s, 4 H), 4.69 (s, 4 H), 5.61 (s, 2 H), 7.91 (q, J = |
| | | 8.1 Hz, 2 H), 8.15 (s, 1 H). |
| | | 504 |
| 13 | C | 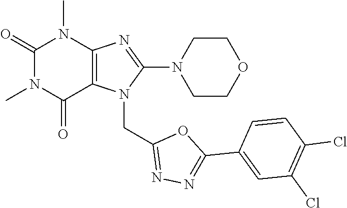 [SMILES: Cn1c(=O)c2c(nc(N3CCOCC3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-1,3- |
| | | dimethyl-8-(morpholin-4-yl)-2,3,6,7-tetrahydro-1H-purine- |
| | | 2,6-dione |
| | | ¹H NMR (400 MHz, CHLOROFORM-d) δ ppm 3.20-3.34 |
| | | (m, 4 H), 3.38 (s, 3 H), 3.57 (s, 3 H), 3.74-3.92 (m, 4 H), |
| | | 5.68 (s, 2 H), 7.60 (d, J = 8.37 Hz, 1 H), 7.86 (dd, J = 8.45, 1.97 |
| | | Hz, 1 H), 8.11 (d, J = 1.90 Hz, 1 H). ¹³C NMR (101 MHz, |
| | | CHLOROFORM-d) δ ppm 28.2, 30.2, 40.3, 51.0, 66.5, |
| | | 105.1, 123.3, 126.3, 129.1, 131.7, 134.1, 137.1, 148.1, |
| | | 151.9, 155.2, 156.4, 162.3, 164.3. |
| | | 492 |
| 14 | C | 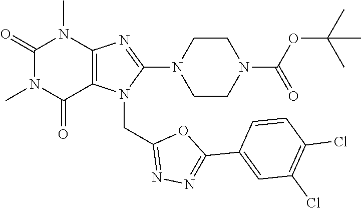 [SMILES: Cn1c(=O)c2c(nc(N3CCN(C(=O)OC(C)(C)C)CC3)n2Cc2nnc(-c3ccc(Cl)c(Cl)c3)o2)n(C)c1=O] |
| | | tert-butyl 4-(7-{[5-(3,4-dichlorophenyl)-1,3,4-oxadiazol-2- |
| | | yl]methyl}-1,3-dimethyl-2,6-dioxo-2,3,6,7-tetrahydro-1H- |
| | | purin-8-yl)piperazine-1-carboxylate |
| | | ¹H NMR (400 MHz, CHLOROFORM-d) δ ppm 1.47 (s, 9 H), |
| | | 3.18-3.31 (m, 4 H), 3.38 (s, 3 H), 3.48-3.63 (m, 7 H), 5.68 |
| | | (s, 2 H), 7.55-7.65 (m, 1 H), 7.87 (dd, J = 8.37, 2.05 Hz, 1 |
| | | H), 8.12 (d, J = 1.90 Hz, 1 H). ¹³C NMR (101 MHz, |
| | | CHLOROFORM-d) δ ppm 27.8, 28.4, 29.9, 40.0, 50.4, 50.4, |
| | | 80.3, 104.8, 123.0, 126.0, 128.8, 131.4, 133.8, 136.8, 147.7, |
| | | 151.6, 154.5, 154.9, 156.1, 161.9, 164.0. |
| | | 591 |
| 15 | C | 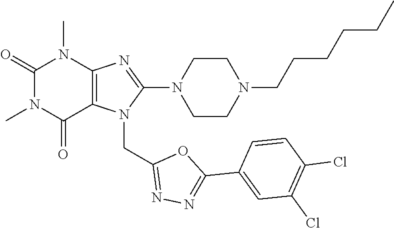 [SMILES: CCCCCCN1CCN(c2nc3c(c(=O)n(C)c(=O)n3C)n2Cc2nnc(-c3ccc(Cl)c(Cl)c3)o2)CC1] |
| | | 7-{[5-(3,4-dichlorophenyl)-1,3,4-oxadiazol-2-yl]methyl}-8-(4- |
| | | hexylpiperazin-1-yl)-1,3-dimethyl-2,3,6,7-tetrahydro-1H- |
| | | purine-2,6-dione |
| | | ¹H NMR (400 MHz, CHLOROFORM-d) δ ppm 0.82-0.95 |
| | | (m, 3 H), 1.30 (br. s., 6 H), 1.56 (br. s., 2 H), 2.48 (br. s., 2 |
| | | H), 2.69 (br. s., 4 H), 3.37 (s, 3 H), 3.44 (br. s., 4 H), 3.56 (s, |
| | | 3 H), 5.64 (s, 2 H), 7.60 (d, J = 8.37 Hz, 1 H), 7.86 (dd, J = 8.37, |
| | | 2.05 Hz, 1 H), 8.11 (d, J = 1.90 Hz, 1 H). ¹³C NMR (101 MHz, |
| | | CHLOROFORM-d) δ ppm 14.0, 22.5, 27.0, 27.8, 29.9, 31.6, |
| | | 40.2, 49.9 (br), 52.1, 58.5, 104.7, 123.0, 126.0, 128.8, 131.3, |
| | | 133.8, 136.7, 147.8, 151.6, 154.8, 157.3, 162.0, 163.9. |
| | | 575 |
| 16 | C | 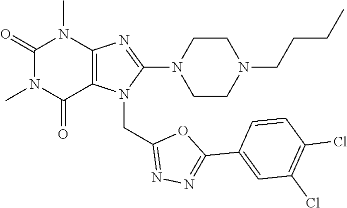 [SMILES: CCCCN1CCN(c2nc3c(c(=O)n(C)c(=O)n3C)n2Cc2nnc(-c3ccc(Cl)c(Cl)c3)o2)CC1] |
| | | 8-(4-butylpiperazin-1-yl)-7-{[5-(3,4-dichlorophenyl)-1,3,4- |
| | | oxadiazol-2-yl]methyl}-1,3-dimethyl-2,3,6,7-tetrahydro-1H- |
| | | purine-2,6-dione |
| | | ¹H NMR (400 MHz, CHLOROFORM-d) δ ppm 0.96 (t, J = 7.35 |
| | | Hz, 3 H), 1.38 (dq, J = 14.89, 7.41 Hz, 2 H), 1.67 (br. s., 4 H), |
| | | 2.67 (br. s., 2 H), 2.91 (br.s., 2 H), 3.37 (s, 3 H), 3.43-3.65 |
| | | (m, 7 H), 5.65 (s, 2 H), 7.61 (d, J = 8.37 Hz, 1 H), 7.87 (dd, |
| | | J = 8.37, 2.05 Hz, 1 H), 8.12 (d, J = 1.90 Hz, 1 H). ¹³C NMR (101 |
| | | MHz, DMSO-d⁶) δ ppm 13.9, 16.6, 20.1, 27.4, 28.2, 29.5, |
| | | 49.7, 52.1, 57.4, 103.9, 123.5, 126.6, 128.1, 132.0, 132.4, |
| | | 135.0, 147.3, 150.9, 153.8, 156.3, 162.6, 163.1 |
| | | 547 |
| 17 | C | 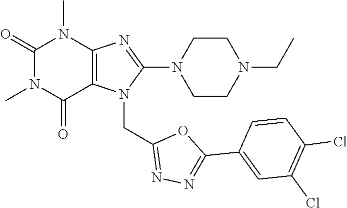 [SMILES: CCN1CCN(c2nc3c(c(=O)n(C)c(=O)n3C)n2Cc2nnc(-c3ccc(Cl)c(Cl)c3)o2)CC1] |
| | | 7-{[5-(3,4-dichlorophenyl)-1,3,4-oxadiazol-2-yl]methyl}-8-(4- |
| | | ethylpiperazin-1-yl)-1,3-dimethyl-2,3,6,7-tetrahydro-1H- |
| | | purine-2,6-dione |
| | | ¹H NMR (400 MHz, CHLOROFORM-d) δ ppm 1.26 (br. s., 3 |
| | | H), 2.71 (br. s., 2 H), 2.77-2.98 (m, 4 H), 3.37 (s, 3 H), 3.56 |
| | | (s, 7 H), 5.65 (s, 2 H), 7.61 (d, J = 8.37 Hz, 1 H), 7.87 (dd, |
| | | J = 8.45, 1.97 Hz, 1 H), 8.12 (d, J = 1.90 Hz, 1 H). ¹³C NMR |
| | | (101 MHz, DMSO-d⁶) δ ppm 11.8, 27.4, 29.6, 49.7, 51.6, |
| | | 51.7 (br), 104.0, 123.5, 126.6, 128.1, 132.0, 132.4, 135.0, |
| | | 147.3, 150.9, 153.8, 156.3, 162.6, 163.1. |
| | | 519 |
| 18 | C | 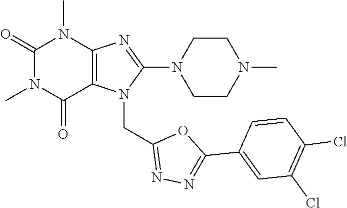 [SMILES: CN1CCN(c2nc3c(c(=O)n(C)c(=O)n3C)n2Cc2nnc(-c3ccc(Cl)c(Cl)c3)o2)CC1] |
| | | 7-{[5-(3,4-dichlorophenyl)-1,3,4-oxadiazol-2-yl]methyl}-1,3- |
| | | dimethyl-8-(4-methylpiperazin-1-yl)-2,3,6,7-tetrahydro-1H- |
| | | purine-2,6-dione |
| | | ¹H NMR (400 MHz, CHLOROFORM-d) δ ppm 2.56 (br. s., 3 |
| | | H), 2.88 (br. s., 4 H), 3.37 (s, 3 H), 3.49-3.65 (m, 7 H), 5.65 |
| | | (s, 2 H), 7.61 (d, J = 8.37 Hz, 1 H), 7.87 (dd, J = 8.45, 1.97 Hz, |
| | | 1 H), 8.12 (d, J = 1.90 Hz, 1 H). ¹³C NMR (101 MHz, DMSO- |
| | | d⁶) δ ppm 27.4, 29.5, 40.7, 45.7, 49.5, 53.9, 103.9, 123.5, |
| | | 126.6, 128.1, 132.0, 132.4, 135.0, 147.3, 150.9, 153.8, |
| | | 156.3, 162.6, 163.1. |
| | | 505 |
| 19 | C | 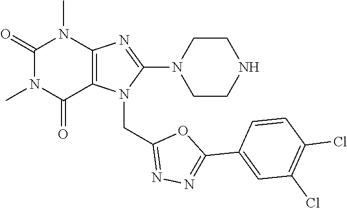 [SMILES: Cn1c(=O)c2c(nc(N3CCNCC3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-1,3- |
| | | dimethyl-8-(piperazin-1-yl)-2,3,6,7-tetrahydro-1H-purine- |
| | | 2,6-dione |
| | | ¹H NMR (400 MHz, DMSO-d⁶) δ ppm 2.70-2.79 (m, 4 H), |
| | | 3.12-3.21 (m, 7 H), 3.40 (s, 3 H), 5.69 (s, 2 H), 7.85-7.96 |
| | | (m, 2 H), 8.15 (dd, J = 1.90, 0.32 Hz, 1 H). ¹³C NMR (101 MHz, |
| | | DMSO-d⁶) δ ppm 27.6, 29.7, 40.6, 45.1, 50.9, 104.1, 123.7, |
| | | 126.8, 128.3, 132.2, 132.7, 135.2, 147.6, 151.1, 154.0, |
| | | 156.8, 162.8, 163.3. |
| | | 491 |
| 20 | C | 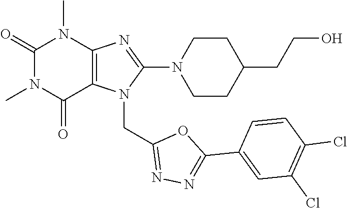 [SMILES: Cn1c(=O)c2c(nc(N3CCC(CCO)CC3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-8-[4- |
| | | (2-hydroxyethyl)piperidin-1-yl]-1,3-dimethyl-2,3,6,7- |
| | | tetrahydro-1H-purine-2,6-dione |
| | | ¹H NMR (400 MHz, DMSO-d⁶) δ ppm 1.13-1.27 (m, 2 H), |
| | | 1.35 (q, J = 6.5 Hz, 2 H), 1.49-1.62 (m, 1 H), 1.64-1.74 (m, |
| | | 2 H), 2.86-2.99 (m, 2 H), 3.40 (s, 3 H), 3.14 (s, 3 H), 3.41- |
| | | 3.46 (m, 2 H), 3.55 (d, J = 12.6 Hz, 2 H), 4.37 (t, J = 5.1 Hz, |
| | | 1 H), 5.66 (s, 2 H), 7.84-7.98 (m, 2 H), 8.14 (d, J = 1.7 Hz, |
| | | 1 H). ¹³C NMR (101 MHz, DMSO-d⁶) δ ppm 27.4, 29.5, 31.4, |
| | | 31.5, 40.2, 40.4, 50.2, 58.1, 103.8, 123.5, 126.6, 128.1, |
| | | 132.0, 132.4, 135.0, 147.5, 150.9, 153.7, 156.9, 162.6, |
| | | 163.2. |
| | | 534 |
| 21 | C | 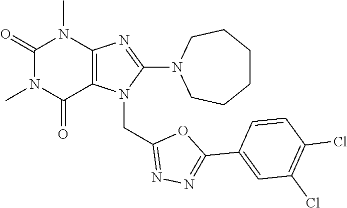 [SMILES: Cn1c(=O)c2c(nc(N3CCCCCC3)n2Cc2nnc(-c3ccc(Cl)c(Cl)c3)o2)n(C)c1=O] |
| | | 8-(azepan-1-yl)-7-{[5-(3,4-dichlorophenyl)-1,3,4-oxadiazol- |
| | | 2-yl]methyl}-1,3-dimethyl-2,3,6,7-tetrahydro-1H-purine-2,6- |
| | | dione |
| | | ¹H NMR (400 MHz, DMSO-d⁶) δ ppm 1.59-1.46 (m, 4 H), |
| | | 1.80-1.67 (m, 4 H), 3.15 (s, 3 H), 3.38 (s, 3 H), 3.61-3.51 |
| | | (m, 4 H), 5.75 (s, 2 H), 7.94-7.83 (m, 2 H), 8.12 (d, J = 1.6 |
| | | Hz, 1 H). ¹³C NMR (101 MHz, CHLOROFORM-d) δ ppm |
| | | 27.2, 27.7, 28.5, 29.8, 40.7, 51.8, 103.6, 123.1, 126.0, 128.7, |
| | | 131.3, 133.7, 136.6, 148.9, 151.8, 154.4, 158.0, 162.4, |
| | | 163.8. |
| | | 504 |
| 22 | C | 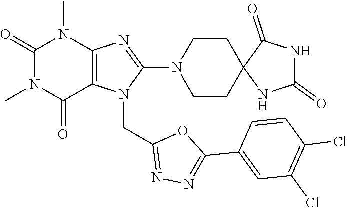 [SMILES: Cn1c(=O)c2c(nc(N3CCC4(CC3)NC(=O)NC4=O)n2Cc2nnc(-c3ccc(Cl)c(Cl)c3)o2)n(C)c1=O] |
| | | 8-(7-{[5-(3,4-dichlorophenyl)-1,3,4-oxadiazol-2-yl]methyl}- |
| | | 1,3-dimethyl-2,6-dioxo-2,3,6,7-tetrahydro-1H-purin-8-yl)- |
| | | 1,3,8-triazaspiro[4.5]decane-2,4-dione |
| | | ¹H NMR (400 MHz, DMSO-d⁶) δ ppm 1.62-1.65 (m, 2 H), |
| | | 1.92-2.04 (m, 2 H), 3.15 (s, 3 H), 3.22-3.33 (m, 2 H), |
| | | 3.41 (s, 3 H), 3.52-3.61 (m, 2 H), 5.70 (s, 2 H), 7.87-7.96 |
| | | (m, 2 H), 8.16 (d, J = 1.9 Hz, 1 H), 8.59 (br s, 1 H), 10.73 (br |
| | | s, 1 H). |
| | | 574 |
| 23 | C⁽²⁾ | 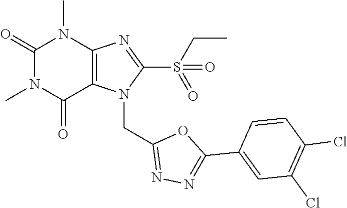 [SMILES: CCS(=O)(=O)c1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazol-2-yl]methyl}-8- |
| | | (ethanesulfonyl)-1,3-dimethyl-2,3,6,7-tetrahydro-1H-purine- |
| | | 2,6-dione |
| | | ¹H NMR (400 MHz, DMSO-d⁶) δ ppm 1.25 (t, J = 7.3 Hz, 3 |
| | | H), 3.23 (s, 3 H), 3.47 (s, 3 H), 3.67 (q, J = 7.4 Hz, 2 H), 6.28 |
| | | (s, 2 H), 7.86-7.98 (m, 2 H), 8.16 (d, J = 1.9 Hz, 1 H). |
| | | 500 |
| 24 | C⁽³⁾ | 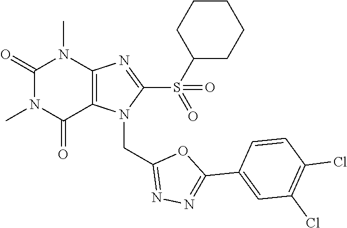 [SMILES: Cn1c(=O)c2c(nc(S(=O)(=O)C3CCCCC3)n2Cc2nnc(-c3ccc(Cl)c(Cl)c3)o2)n(C)c1=O] |
| | | 8-(cyclohexanesulfonyl)-7-{[5-(3,4-dichlorophenyl)-1,3,4- |
| | | oxadiazol-2-yl]methyl}-1,3-dimethyl-2,3,6,7-tetrahydro-1H- |
| | | purine-2,6-dione |
| | | ¹H NMR (400 MHz, DMSO-d⁶) δ ppm 1.47-1.07 (m, 4 H), |
| | | 1.64-1.53 (m, 2 H), 1.81-1.70 (m, 2 H), 2.02-1.91 (m, 2 |
| | | H), 3.22 (s, 3 H), 3.48 (s, 3 H), 3.69-3.55 (m, 1 H), 6.27 (s, |
| | | 2 H), 7.98-7.86 (m, 2 H), 8.16 (dd, J = 0.6, 1.8 Hz, 1 H). |
| | | 554 |
| 25 | C | 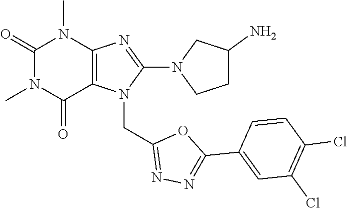 [SMILES: Cn1c(=O)c2c(nc(N3CCC(N)C3)n2Cc2nnc(-c3ccc(Cl)c(Cl)c3)o2)n(C)c1=O] |
| | | 8-(3-aminopyrrolidin-1-yl)-7-{[5-(3,4-dichlorophenyl)-1,3,4- |
| | | oxadiazol-2-yl]methyl}-1,3-dimethyl-2,3,6,7-tetrahydro-1H- |
| | | purine-2,6-dione |
| | | ¹H NMR (400 MHz, CHLOROFORM-d) δ ppm 1.84-2.02 |
| | | (m, 1 H), 2.22 (d, J = 7.58 Hz, 1 H), 3.37 (s, 3 H), 3.48-3.60 |
| | | (m, 4 H), 3.70 (ddd, J = 9.64, 8.29, 5.45 Hz, 1 H), 3.78-3.98 |
| | | (m, 3 H), 5.78-5.94 (m, 2 H), 7.56 (d, J = 8.37 Hz, 1 H), 7.78 |
| | | (dd, J = 8.45, 1.97 Hz, 1 H), 8.04 (d, J = 1.90 Hz, 1 H). |
| | | 491 |
| 26 | C | 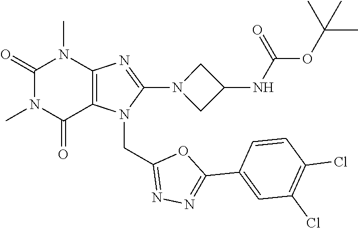 [SMILES: Cn1c(=O)c2c(nc(N3CC(NC(=O)OC(C)(C)C)C3)n2Cc2nnc(-c3ccc(Cl)c(Cl)c3)o2)n(C)c1=O] |
| | | tert-butyl N-[1-(7-{[5-(3,4-dichlorophenyl)-1,3,4-oxadiazol-2- |
| | | yl]methyl}-1,3-dimethyl-2,6-dioxo-2,3,6,7-tetrahydro-1H- |
| | | purin-8-yl)azetidin-3-yl]carbamate |
| | | ¹H NMR (400 MHz, CHLOROFORM-d) δ ppm 1.44 (s, 9 H), |
| | | 3.38 (s, 3 H), 3.53 (s, 3 H), 4.13 (dd, J = 8.53, 5.84 Hz, 2 H), |
| | | 4.51 (t, J = 7.98 Hz, 2 H), 5.62 (s, 2 H), 7.59 (d, J = 8.37 Hz, 1 |
| | | H), 7.84 (dd, J = 8.37, 2.05 Hz, 1 H), 8.10 (d, J = 2.05 Hz, 1 H). |
| | | 577 |
| 27 | C | 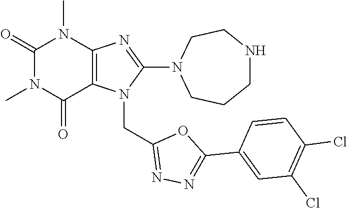 [SMILES: Cn1c(=O)c2c(nc(N3CCCNCC3)n2Cc2nnc(-c3ccc(Cl)c(Cl)c3)o2)n(C)c1=O] |
| | | 8-(1,4-diazepan-1-yl)-7-{[5-(3,4-dichlorophenyl)-1,3,4- |
| | | oxadiazol-2-yl]methyl}-1,3-dimethyl-2,3,6,7-tetrahydro-1H- |
| | | purine-2,6-dione |
| | | ¹H NMR (400 MHz, CHLOROFORM-d) δ ppm 2.27 (br. s., 2 |
| | | H), 3.38 (s, 3 H), 3.40-3.48 (m, 2 H), 3.50-3.59 (m, 5 H), |
| | | 3.63-3.75 (m, 2 H), 3.85 (br. s., 2 H), 5.75 (s, 2 H), 7.56- |
| | | 7.65 (m, 1 H), 7.81-7.90 (m, 1 H), 8.10 (d, J = 1.90 Hz, 1 H). |
| | | 505 |
| 28 | C | 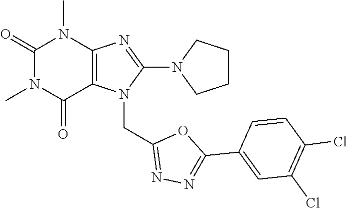 [SMILES: Cn1c(=O)c2c(nc(N3CCCC3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-1,3- |
| | | dimethyl-8-(pyrrolidin-1-yl)-2,3,6,7-tetrahydro-1H-purine- |
| | | 2,6-dione |
| | | ¹H NMR (400 MHz, CHLOROFORM-d) δ ppm 1.89-2.09 |
| | | (m, 4 H), 3.38 (s, 3 H), 3.54 (s, 3 H), 3.68 (t, J = 6.56 Hz, 4 H), |
| | | 5.85 (s, 2 H), 7.58 (d, J = 8.37 Hz, 1 H), 7.81 (dd, J = 8.37, 1.90 |
| | | Hz, 1 H), 8.07 (d, J = 1.90 Hz, 1 H). |
| | | 476 |
| 29 | C | 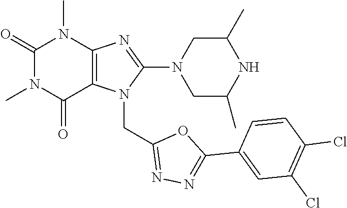 [SMILES: CC1CN(c2nc3c(c(=O)n(C)c(=O)n3C)n2Cc2nnc(-c3ccc(Cl)c(Cl)c3)o2)CC(C)N1] |
| | | 7-{[5-(3,4-dichlorophenyl)-1,3,4-oxadiazol-2-yl]methyl}-8- |
| | | (3,5-dimethylpiperazin-1-yl)-1,3-dimethyl-2,3,6,7- |
| | | tetrahydro-1H-purine-2,6-dione |
| | | ¹H NMR (400 MHz, CHLOROFORM-d) δ ppm 1.09-1.24 |
| | | (m, 6 H), 1.28 (d, J = 6.00 Hz, 1 H), 2.84 (br. s., 2 H), 3.15 (br. |
| | | s., 2 H), 3.37 (s, 3 H), 3.44 (d, J = 12.16 Hz, 2 H), 3.56 (s, 3 |
| | | H), 5.65 (s, 2 H), 7.60 (d, J = 8.37 Hz, 1 H), 7.77-7.94 (m, 1 |
| | | H), 8.11 (s, 1 H). |
| | | 519 |
| 30 | C | 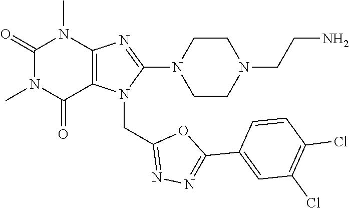 [SMILES: Cn1c(=O)c2c(nc(N3CCN(CCN)CC3)n2Cc2nnc(-c3ccc(Cl)c(Cl)c3)o2)n(C)c1=O] |
| | | 8-[4-(2-aminoethyl)piperazin-1-yl]-7-{[5-(3,4- |
| | | dichlorophenyl)-1,3,4-oxadiazol-2-yl]methyl}-1,3-dimethyl- |
| | | 2,3,6,7-tetrahydro-1H-purine-2,6-dione |
| | | ¹H NMR (400 MHz, CHLOROFORM-d) δ ppm 2.52-2.69 |
| | | (m, 6 H), 2.99 (t, J = 5.77 Hz, 2 H), 3.25-3.44 (m, 7 H), 3.51- |
| | | 3.61 (m, 3 H), 5.55-5.72 (m, 2 H), 7.55-7.65 (m, 1 H), |
| | | 7.79-7.89 (m, 1 H), 8.04-8.14 (m, 1 H). |
| | | 534 |
| 31 | C | 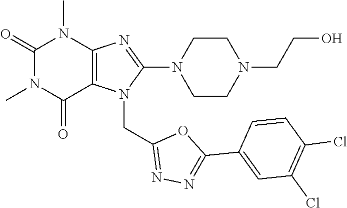 [SMILES: Cn1c(=O)c2c(nc(N3CCN(CCO)CC3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-8-[4- |
| | | (2-hydroxyethyl)piperazin-1-yl]-1,3-dimethyl-2,3,6,7- |
| | | tetrahydro-1H-purine-2,6-dione |
| | | ¹H NMR (400 MHz, CHLOROFORM-d) δ ppm 2.64-2.74 |
| | | (m, 2 H), 2.74-2.84 (m, 4 H), 3.37 (s, 3 H), 3.39-3.50 (m, |
| | | 4 H), 3.56 (s, 3 H), 3.71 (t, J = 5.13 Hz, 2 H), 5.66 (s, 2 H), |
| | | 7.60 (d, J = 8.37 Hz, 1 H), 7.85 (dd, J = 8.37, 2.05 Hz, 1 H), |
| | | 8.10 (d, J = 1.90 Hz, 1 H). |
| | | 535 |
| 32 | C | 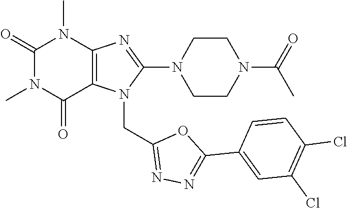 [SMILES: CC(=O)N1CCN(c2nc3c(c(=O)n(C)c(=O)n3C)n2Cc2nnc(-c3ccc(Cl)c(Cl)c3)o2)CC1] |
| | | 8-(4-acetylpiperazin-1-yl)-7-{[5-(3,4-dichlorophenyl)-1,3,4- |
| | | oxadiazol-2-yl]methyl}-1,3-dimethyl-2,3,6,7-tetrahydro-1H- |
| | | purine-2,6-dione |
| | | ¹H NMR (400 MHz, CHLOROFORM-d) δ ppm 2.15 (s, 3 H), |
| | | 3.31 (br. s., 4 H), 3.37 (s, 3 H), 3.55 (s, 3 H), 3.69 (br. s., 4 |
| | | H), 5.69 (s, 2 H), 7.60 (d, J = 7.58 Hz, 1 H), 7.86 (d, J = 7.11 |
| | | Hz, 1 H), 8.11 (s, 1 H). |
| | | 533 |
| 33 | C | 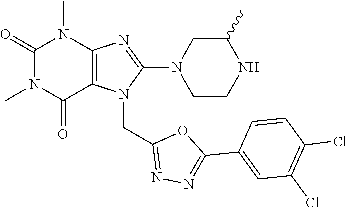 [SMILES: CC1CN(c2nc3c(c(=O)n(C)c(=O)n3C)n2Cc2nnc(-c3ccc(Cl)c(Cl)c3)o2)CCN1] |
| | | 7-{[5-(3,4-dichlorophenyl)-1,3,4-oxadiazol-2-yl]methyl}-1,3- |
| | | dimethyl-8-(3-methylpiperazin-1-yl)-2,3,6,7-tetrahydro-1H- |
| | | purine-2,6-dione |
| | | ¹H NMR (400 MHz, CHLOROFORM-d) δ ppm 1.23 (d, |
| | | J = 6.48 Hz, 3 H), 2.92-3.03 (m, 1 H), 3.04-3.23 (m, 3 H), |
| | | 3.24-3.34 (m, 1 H), 3.37 (s, 3 H), 3.43-3.53 (m, 2 H), 3.56 |
| | | (s, 3 H), 5.65 (s, 2 H), 7.56-7.65 (m, 1 H), 7.82-7.91 (m, 1 |
| | | H), 8.12 (s, 1 H). |
| | | 505 |
| 34 | C | 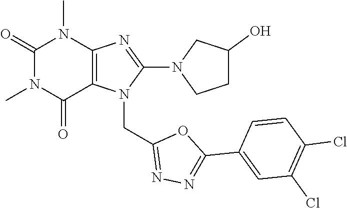 [SMILES: Cn1c(=O)c2c(nc(N3CCC(O)C3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-8-(3- |
| | | hydroxypyrrolidin-1-yl)-1,3-dimethyl-2,3,6,7-tetrahydro-1H- |
| | | purine-2,6-dione |
| | | ¹H NMR (400 MHz, CHLOROFORM-d) δ ppm 2.02-2.19 |
| | | (m, 3 H), 3.39 (s, 3 H), 3.57 (s, 3 H), 3.72-3.82 (m, 2 H), |
| | | 3.82-3.90 (m, 1 H), 3.90-4.02 (m, 1 H), 4.53-4.66 (m, 1 |
| | | H), 5.81 (d, J = 17.38 Hz, 1 H), 5.92 (d, J = 17.38 Hz, 1 H), 7.59 |
| | | (d, J = 8.37 Hz, 1 H), 7.83 (dd, J = 8.45, 1.97 Hz, 1 H), 8.09 (d, |
| | | J = 1.90 Hz, 1 H). |
| | | 492 |
| 35 | C | 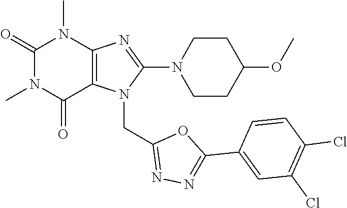 [SMILES: COC1CCN(c2nc3c(c(=O)n(C)c(=O)n3C)n2Cc2nnc(-c3ccc(Cl)c(Cl)c3)o2)CC1] |
| | | 7-{[5-(3,4-dichlorophenyl)-1,3,4-oxadiazol-2-yl]methyl}-8-(3- |
| | | hydroxyazetidin-1-yl)-1,3-dimethyl-2,3,6,7-tetrahydro-1H- |
| | | purine-2,6-dione |
| | | ¹H NMR (400 MHz, DMSO-d⁶) δ ppm 3.16 (s, 3 H), 3.38 (s, |
| | | 3 H), 4.01 (dd, J = 8.92, 4.98 Hz, 2 H), 4.42 (dd, J = 8.21, 7.27 |
| | | Hz, 2 H), 4.50-4.65 (m, 1 H), 5.63 (s, 2 H), 5.81 (d, J = 6.32 |
| | | Hz, 1 H), 7.86-7.98 (m, 2 H), 8.16 (dd, J = 1.90, 0.47 Hz, 1 |
| | | H). |
| | | 478 |
| 36 | C | 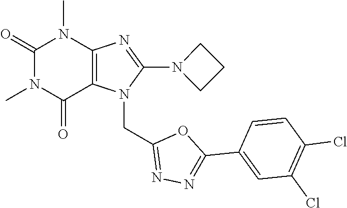 [SMILES: Cn1c(=O)c2c(nc(N3CCC3)n2Cc2nnc(-c3ccc(Cl)c(Cl)c3)o2)n(C)c1=O] |
| | | 8-(azetidin-1-yl)-7-{[5-(3,4-dichlorophenyl)-1,3,4-oxadiazol- |
| | | 2-yl]methyl}-1,3-dimethyl-2,3,6,7-tetrahydro-1H-purine-2,6- |
| | | dione |
| | | ¹H NMR (400 MHz, CHLOROFORM-d) δ ppm 2.46 (t, J = 7.74 |
| | | Hz, 2 H), 3.39 (s, 3 H), 3.51-3.57 (m, 3 H), 4.22-4.35 (m, |
| | | 4 H), 5.63 (s, 2 H), 7.59 (d, J = 8.37 Hz, 1 H), 7.84 (dd, J = 8.37, |
| | | 2.05 Hz, 1 H), 8.09 (d, J = 1.89 Hz, 1 H) |
| | | 462 |
| 37 | C | 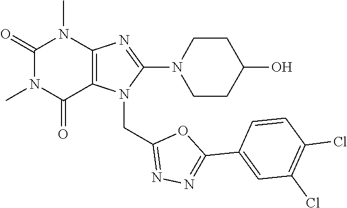 [SMILES: Cn1c(=O)c2c(nc(N3CCC(O)CC3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-8-(4- |
| | | hydroxypiperidin-1-yl)-1,3-dimethyl-2,3,6,7-tetrahydro-1H- |
| | | purine-2,6-dione |
| | | ¹H NMR (400 MHz, CHLOROFORM-d) δ ppm 1.67-1.75 |
| | | (m, 2 H), 1.93-2.06 (m, 2 H), 3.15 (ddd, J = 12.79, 9.32, 3.16 |
| | | Hz, 2 H), 3.37 (s, 3 H), 3.47-3.61 (m, 5 H), 3.93 (dt, J = 8.21, |
| | | 4.26 Hz, 1 H), 5.64 (s, 2 H), 7.55-7.65 (m, 1 H), 7.86 (dd, |
| | | J = 8.37, 2.05 Hz, 1 H), 8.11 (d, J = 2.05 Hz, 1 H) |
| | | 506 |
| 38 | C | 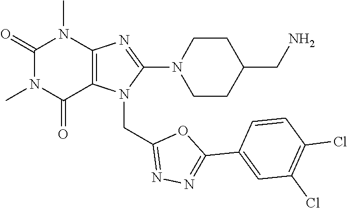 [SMILES: Cn1c(=O)c2c(nc(N3CCC(CN)CC3)n2Cc2nnc(-c3ccc(Cl)c(Cl)c3)o2)n(C)c1=O] |
| | | 8-[4-(aminomethyl)piperidin-1-yl]-7-{[5-(3,4-dichlorophenyl)- |
| | | 1,3,4-oxadiazol-2-yl)methyl}-1,3-dimethyl-2,3,6,7- |
| | | tetrahydro-1H-purine-2,6-dione |
| | | ¹H NMR (400 MHz, DMSO-d⁶) δ ppm 1.16 (qd, J = 12.3, 3.6 |
| | | Hz, 2 H), 1.32-1.43 (m, 1 H), 1.69-1.77 (m, 2 H), 2.40 (d, |
| | | J = 6.3 Hz, 2 H), 2.92 (td, J = 12.2, 2.1 Hz, 2 H), 3.15 (s, 3 |
| | | H), 3.40 (s, 3 H), 3.57 (d, J = 12.3 Hz, 2 H), 5.66 (s, 2 H), |
| | | 7.88-7.91 (m, 1 H), 7.92-7.95 (m, 1 H), 8.15 (d, J = 1.9 Hz, |
| | | 1 H) |
| | | 519 |
| 39 | C | 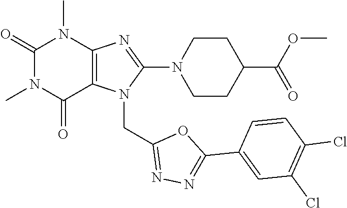 [SMILES: COC(=O)C1CCN(c2nc3c(c(=O)n(C)c(=O)n3C)n2Cc2nnc(-c3ccc(Cl)c(Cl)c3)o2)CC1] |
| | | methyl 1-(7-{[5-(3,4-dichlorophenyl)-1,3,4-oxadiazol-2- |
| | | yl]methyl}-1,3-dimethyl-2,6-dioxo-2,3,6,7-tetrahydro-1H- |
| | | purin-8-yl)piperidine-4-carboxylate |
| | | ¹H NMR (400 MHz, CHLOROFORM-d) δ ppm 1.79-1.94 |
| | | (m, 2 H), 1.96-2.10 (m, 2 H), 2.43-2.60 (m, 1 H), 3.01- |
| | | 3.15 (m, 2 H), 3.36 (s, 3 H), 3.47-3.60 (m, 5 H), 3.70 (s, 3 |
| | | H), 5.62 (s, 2 H), 7.60 (d, J = 8.37 Hz, 1 H), 7.86 (dd, J = 8.37, |
| | | 2.05 Hz, 1 H), 8.11 (d, J = 1.90 Hz, 1 H) |
| | | 548 |
| 40 | C | 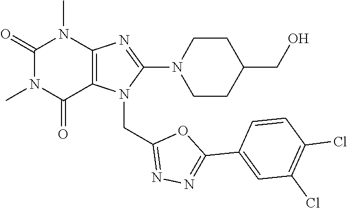 [SMILES: Cn1c(=O)c2c(nc(N3CCC(CO)CC3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-8-[4- |
| | | (hydroxymethyl)piperidin-1-yl]-1,3-dimethyl-2,3,6,7- |
| | | tetrahydro-1H-purine-2,6-dione |
| | | ¹H NMR (400 MHz, CHLOROFORM-d) δ ppm 1.39 (qd, |
| | | J = 12.43, 4.11 Hz, 2 H), 1.66-1.78 (m, 1 H), 1.78-1.89 (m, |
| | | 2 H), 3.04 (td, J = 12.48, 2.37 Hz, 2 H), 3.37 (s, 3 H), 3.52- |
| | | 3.61 (m, 7 H), 5.64 (s, 2 H), 7.60 (d, J = 8.37 Hz, 1 H), 7.86 |
| | | (dd, J = 8.37, 2.05 Hz, 1 H), 8.11 (d, J = 1.90 Hz, 1 H) |
| | | 520 |
| 41 | C | 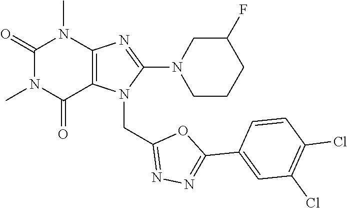 [SMILES: Cn1c(=O)c2c(nc(N3CCCC(F)C3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-8-(3- |
| | | fluoropiperidin-1-yl)-1,3-dimethyl-2,3,6,7-tetrahydro-1H- |
| | | purine-2,6-dione |
| | | ¹H NMR (400 MHz, CHLOROFORM-d) d ppm 1.60-1.74 |
| | | (m, 2 H), 1.92-2.09 (m, 2 H), 3.09-3.23 (m, 1 H), 3.28- |
| | | 3.47 (m, 5 H), 3.49-3.63 (m, 4 H), 4.73 (dt, J = 5.17, 2.55 Hz, |
| | | 1 H), 4.85 (dt, J = 5.09, 2.59 Hz, 1 H), 5.55-5.75 (m, 2 H), |
| | | 7.60 (d, J = 8.37 Hz, 1 H), 7.87 (dd, J = 8.37, 2.05 Hz, 1 H), |
| | | 8.13 (d, J = 1.74 Hz, 1 H). |
| | | 508 |
| 42 | C | 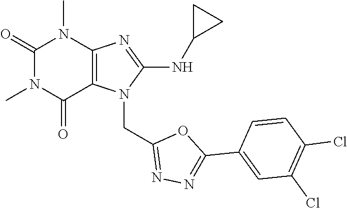 [SMILES: Cn1c(=O)c2c(nc(NC3CC3)n2Cc2nnc(-c3ccc(Cl)c(Cl)c3)o2)n(C)c1=O] |
| | | 8-(cyclopropylamino)-7-{[5-(3,4-dichlorophenyl)-1,3,4- |
| | | oxadiazol-2-yl]methyl}-1,3-dimethyl-2,3,6,7-tetrahydro-1H- |
| | | purine-2,6-dione |
| | | ¹H NMR (400 MHz, CHLOROFORM-d) δ ppm 2.72 (t, J = 5.37 |
| | | Hz, 2 H), 3.43 (s, 3 H), 3.56 (s, 4 H), 3.97-4.08 (m, 2 H), |
| | | 6.01 (s, 2 H), 7.58 (d, J = 8.37 Hz, 1 H), 7.82 (dd, J = 8.45, 1.97 |
| | | Hz, 1 H), 8.07 (d, J = 1.90 Hz, 1 H). |
| | | 462 |
| 43 | C | 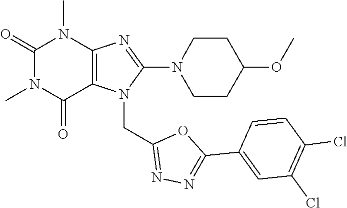 [SMILES: COC1CCN(c2nc3c(c(=O)n(C)c(=O)n3C)n2Cc2nnc(-c3ccc(Cl)c(Cl)c3)o2)CC1] |
| | | 7-{[5-(3,4-dichlorophenyl)-1,3,4-oxadiazol-2-yl]methyl}-8-(4- |
| | | methoxypiperidin-1-yl)-1,3-dimethyl-2,3,6,7-tetrahydro-1H- |
| | | purine-2,6-dione |
| | | ¹H NMR (400 MHz, CHLOROFORM-d) δ ppm 1.67-1.74 |
| | | (m, 2 H), 1.97 (ddt, J = 12.99, 6.87, 3.34, 3.34 Hz, 2 H), 3.13 |
| | | (ddd, J = 12.56, 8.77, 3.32 Hz, 2 H), 3.34-3.39 (m, 6 H), 3.41 |
| | | (td, J = 7.70, 3.87 Hz, 1 H), 3.45-3.53 (m, 2 H), 3.56 (s, 3 H), |
| | | 5.63 (s, 2 H), 7.60 (d, J = 8.37 Hz, 1 H), 7.86 (dd, J = 8.45, 1.97 |
| | | Hz, 1 H), 8.11 (d, J = 1.74 Hz, 1 H) |
| | | 520 |
| 44 | C | 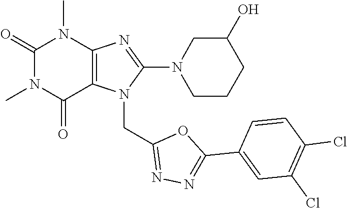 [SMILES: Cn1c(=O)c2c(nc(N3CCCC(O)C3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}-8-(3- |
| | | hydroxypiperidin-1-yl)-1,3-dimethyl-2,3,6,7-tetrahydro-1H- |
| | | purine-2,6-dione |
| | | ¹H NMR (400 MHz, CHLOROFORM-d) δ ppm 1.54-1.66 |
| | | (m, 1 H), 1.73-1.87 (m, 2 H), 1.93-2.08 (m, 1 H), 3.25 (ddd, |
| | | J = 12.79, 9.95, 3.16 Hz, 1 H), 3.33-3.53 (m, 6 H), 3.55-3.59 |
| | | (m, 3 H), 3.93-4.05 (m, 1 H), 5.57-5.81 (m, 2 H), 7.60 (d, |
| | | J = 8.53 Hz, 1 H), 7.86 (dd, J = 8.45, 1.97 Hz, 1 H), 8.11 (d, |
| | | J = 1.74 Hz, 1 H) |
| | | 506 |
| 45 | C | 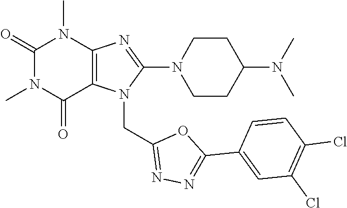 [SMILES: CN(C)C1CCN(c2nc3c(c(=O)n(C)c(=O)n3C)n2Cc2nnc(-c3ccc(Cl)c(Cl)c3)o2)CC1] |
| | | 7-{[5-(3,4-dichlorophenyl)-1,3,4-oxadiazol-2-yl]methyl}-8-[4- |
| | | (dimethylamino)piperidin-1-yl]-1,3-dimethyl-2,3,6,7- |
| | | tetrahydro-1H-purine-2,6-dione |
| | | ¹H NMR (400 MHz, CHLOROFORM-d) δ ppm 1.75 (d, |
| | | J = 10.58 Hz, 2 H), 2.02 (d, J = 11.85 Hz, 2 H), 2.47 (br. s., 7 |
| | | H), 3.00-3.10 (m, 2 H), 3.35-3.39 (m, 3 H), 3.56 (s, 3 H), |
| | | 3.62 (d, J = 13.11 Hz, 2 H), 5.64 (s, 2 H), 7.61 (d, J = 8.37 Hz, |
| | | 1 H), 7.87 (dd, J = 8.37, 2.05 Hz, 1 H), 8.12 (d, J = 1.90 Hz, 1 |
| | | H) |
| | | 533 |
| 46 | C | 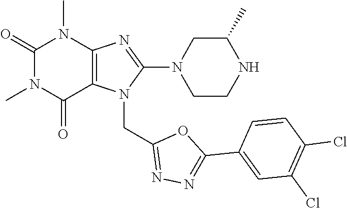 [SMILES: C[C@H]1CN(c2nc3c(c(=O)n(C)c(=O)n3C)n2Cc2nnc(-c3ccc(Cl)c(Cl)c3)o2)CCN1] |
| | | 7-{[5-(3,4-dichlorophenyl)-1,3,4-oxadiazol-2-yl]methyl}-1,3- |
| | | dimethyl-8-[(3S)-3-methylpiperazin-1-yl]-2,3,6,7-tetrahydro- |
| | | 1H-purine-2,6-dione |
| | | ¹H NMR (400 MHz, CHLOROFORM-d) δ ppm 1.14 (d, |
| | | J = 6.32 Hz, 3 H), 2.82 (dd, J = 12.24, 10.35 Hz, 1 H), 2.95- |
| | | 3.24 (m, 4 H), 3.33-3.49 (m, 5 H), 3.54-3.60 (m, 3 H), 5.59- |
| | | 5.71 (m, 2 H), 7.56-7.64 (m, 1 H), 7.80-7.93 (m, 1 H), |
| | | 8.11 (d, J = 1.90 Hz, 1 H) |
| | | 505 |
| 47 | C | 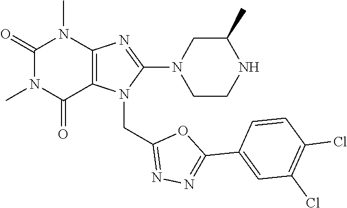 [SMILES: C[C@@H]1CN(c2nc3c(c(=O)n(C)c(=O)n3C)n2Cc2nnc(-c3ccc(Cl)c(Cl)c3)o2)CCN1] |
| | | 7-{[5-(3,4-dichlorophenyl)-1,3,4-oxadiazol-2-yl]methyl}-1,3- |
| | | dimethyl-8-[(3R)-3-methylpiperazin-1-yl]-2,3,6,7-tetrahydro- |
| | | 1H-purine-2,6-dione |
| | | ¹H NMR (400 MHz, CHLOROFORM-d) δ ppm 1.14 (d, |
| | | J = 6.32 Hz, 3 H), 2.81 (dd, J = 12.32, 10.43 Hz, 1 H), 2.95- |
| | | 3.23 (m, 4 H), 3.35-3.48 (m, 5 H), 3.52-3.61 (m, 3 H), 5.58- |
| | | 5.71 (m, 2 H), 7.60 (d, J = 8.37 Hz, 1 H), 7.86 (dd, J = 8.45, |
| | | 1.97 Hz, 1 H), 8.11 (d, J = 1.90 Hz, 1 H) |
| | | 505 |
| 48 | C | 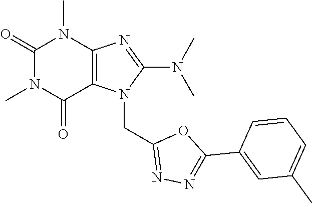 [SMILES: Cc1cccc(-c2nnc(Cn3c(N(C)C)nc4c3c(=O)n(C)c(=O)n4C)o2)c1] |
| | | 8-(dimethylamino)-1,3-dimethyl-7-{[5-(3-methylphenyl)- |
| | | 1,3,4-oxadiazol-2-yl]methyl}-2,3,6,7-tetrahydro-1H-purine- |
| | | 2,6-dione |
| | | ¹H NMR (400 MHz, CHLOROFORM-d) δ ppm 2.42 (s, 3 H), |
| | | 2.98-3.11 (m, 6 H), 3.38 (s, 3 H), 3.55 (s, 3 H), 5.73 (s, 2 |
| | | H), 7.30-7.46 (m, 2 H), 7.73-7.80 (m, 1 H), 7.80-7.86 (m, |
| | | 1 H). |
| | | 396 |
| 49 | C | 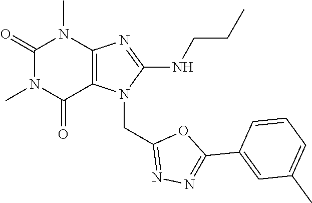 [SMILES: CCCNc1nc2c(c(=O)n(C)c(=O)n2C)n1Cc1nnc(-c2cccc(C)c2)o1] |
| | | 1,3-dimethyl-7-{[5-(3-methylphenyl)-1,3,4-oxadiazol-2- |
| | | yl]methyl}-8-(propylamino)-2,3,6,7-tetrahydro-1H-purine- |
| | | 2,6-dione |
| | | ¹H NMR (400 MHz, CHLOROFORM-d) δ ppm 1.00 (t, J = 7.42 |
| | | Hz, 3 H), 1.62-1.77 (m, 2 H), 2.43 (s, 3 H), 3.42 (s, 3 H), |
| | | 3.47 (t, J = 6.95 Hz, 2 H), 3.55 (s, 3 H), 5.71 (s, 2 H), 7.32- |
| | | 7.44 (m, 2 H), 7.79-7.91 (m, 2 H) |
| | | 410 |
| 50 | C | 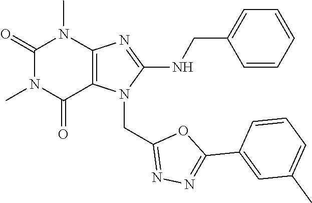 [SMILES: Cc1cccc(-c2nnc(Cn3c(NCc4ccccc4)nc4c3c(=O)n(C)c(=O)n4C)o2)c1] |
| | | 8-(benzylamino)-1,3-dimethyl-7-{[5-(3-methylphenyl)-1,3,4- |
| | | oxadiazo-2-yl]methyl}-2,3,6,7-tetrahydro-1H-purine-2,6- |
| | | dione |
| | | ¹H NMR (400 MHz, CHLOROFORM-d) δ ppm 2.43 (s, 3 H), |
| | | 3.42 (s, 3 H), 3.54 (s, 3 H), 4.62-4.75 (m, 2 H), 5.72 (s, 2 |
| | | H), 5.93 (br. s., 1 H), 7.27-7.45 (m, 7 H), 7.77-7.90 (m, 2 |
| | | H) |
| | | 458 |
| 51 | C | 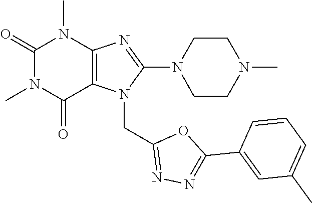 [SMILES: Cc1cccc(-c2nnc(Cn3c(N4CCN(C)CC4)nc4c3c(=O)n(C)c(=O)n4C)o2)c1] |
| | | 1,3-dimethyl-7-{[5-(3-methylphenyl)-1,3,4-oxadiazol-2- |
| | | yl]methyl}-8-(4-methylpiperazin-1-yl)-2,3,6,7-tetrahydro-1H- |
| | | purine-2,6-dione |
| | | ¹H NMR (400 MHz, CHLOROFORM-d) δ ppm 2.36 (s, 3 H) |
| | | 2.42 (s, 3 H) 2.54-2.63 (m, 4 H) 3.34-3.40 (m, 7 H) 3.55 |
| | | (s, 3 H) 5.65 (s, 2 H) 7.31-7.42 (m, 2 H) 7.74-7.81 (m, 1 |
| | | H) 7.82-7.86 (m, 1 H) |
| | | 451 |
| 52 | C | 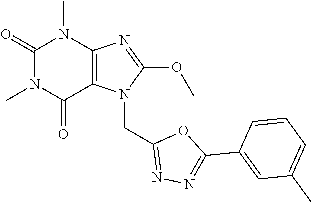 [SMILES: COc1nc2c(c(=O)n(C)c(=O)n2C)n1Cc1nnc(-c2cccc(C)c2)o1] |
| | | 8-methoxy-1,3-dimethyl-7-{[5-(3-methylphenyl)-1,3,4- |
| | | oxadiazol-2-yl]methyl}-2,3,6,7-tetrahydro-1H-purine-2,6- |
| | | dione |
| | | ¹H NMR (400 MHz, CHLOROFORM-d) δ ppm 2.42 (s, 3 H) |
| | | 3.39 (s, 3 H) 3.56 (s, 3 H) 4.17 (s, 3 H) 5.64 (s, 2 H) 7.30- |
| | | 7.45 (m, 2 H) 7.78 (d, J = 7.07 Hz, 1 H) 7.84 (s, 1 H) |
| | | 383 |
| 53 | C | 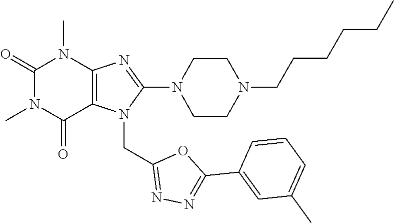 [SMILES: CCCCCCN1CCN(c2nc3c(c(=O)n(C)c(=O)n3C)n2Cc2nnc(-c3cccc(C)c3)o2)CC1] |
| | | 8-(4-hexylpiperazin-1-yl)-1,3-dimethyl-7-{[5-(3- |
| | | methylphenyl)-1,3,4-oxadiazol-2-yl]methyl}-2,3,6,7- |
| | | tetrahydro-1H-purine-2,6-dione |
| | | ¹H NMR (400 MHz, CHLOROFORM-d) δ ppm 0.82-0.94 |
| | | (m, 3 H) 1.26-1.39 (m, 6 H) 1.68-1.83 (m, 2 H) 2.43 (s, 3 |
| | | H) 2.99-3.10 (m, 2 H) 3.13 (br. s., 2 H) 3.37 (s, 3 H) 3.54 (s, |
| | | 3 H) 3.59-3.77 (m, 6 H) 5.69 (s, 2 H) 7.33-7.46 (m, 2 H) |
| | | 7.74-7.80 (m, 1 H) 7.82 (s, 1 H) |
| | | 521 |
| 54 | C | 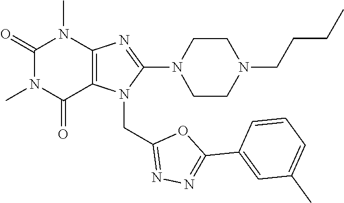 [SMILES: CCCCN1CCN(c2nc3c(c(=O)n(C)c(=O)n3C)n2Cc2nnc(-c3cccc(C)c3)o2)CC1] |
| | | 8-(4-butylpiperazin-1-yl)-1,3-dimethyl-7-{[5-(3- |
| | | methylphenyl)-1,3,4-oxadiazol-2-yl]methyl}-2,3,6,7- |
| | | tetrahydro-1H-purine-2,6-dione |
| | | ¹H NMR (400 MHz, CHLOROFORM-d) δ ppm 0.98 (t, J = 7.35 |
| | | Hz, 3 H), 1.36-1.47 (m, 2 H), 1.83-1.91 (m, 4 H), 2.44 (s, |
| | | 3 H), 2.96-3.03 (m, 2 H), 3.12 (br. s., 2 H), 3.35-3.39 (m, |
| | | 3 H), 3.55 (s, 3 H), 3.66 (d, J = 13.43 Hz, 2 H), 4.03 (d, |
| | | J = 12.95 Hz, 2 H), 5.69 (s, 2 H), 7.35-7.45 (m, 2 H), 7.80 |
| | | (dt, J = 6.87, 1.62 Hz, 1 H), 7.83-7.87 (m, 1 H). |
| | | 493 |
| 55 | C | 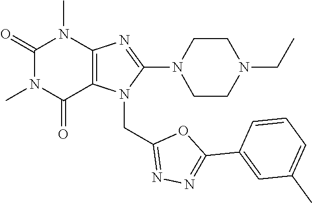 [SMILES: CCN1CCN(c2nc3c(c(=O)n(C)c(=O)n3C)n2Cc2nnc(-c3cccc(C)c3)o2)CC1] |
| | | 8-(4-ethylpiperazin-1-yl)-1,3-dimethyl-7-{[5-(3- |
| | | methylphenyl)-1,3,4-oxadiazol-2-yl]methyl}-2,3,6,7- |
| | | tetrahydro-1H-purine-2,6-dione |
| | | ¹H NMR (400 MHz, CHLOROFORM-d) δ ppm 1.09 (t, J = 7.27 |
| | | Hz, 3 H) 2.37-2.50 (m, 5 H) 2.50-2.60 (m, 4 H) 3.29-3.43 |
| | | (m, 7 H) 3.56 (s, 3 H) 5.66 (s, 2 H) 7.31-7.45 (m, 2 H) 7.78 |
| | | (d, J = 7.11 Hz, 1 H) 7.84 (s, 1 H) |
| | | 465 |
| 56 | C | 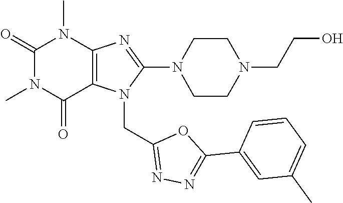 [SMILES: Cc1cccc(-c2nnc(Cn3c(N4CCN(CCO)CC4)nc4c3c(=O)n(C)c(=O)n4C)o2)c1] |
| | | 8-[4-(2-hydroxyethyl)piperazin-1-yl]-1,3-dimethyl-7-{[5-(3- |
| | | methylphenyl)-1,3,4-oxadiazol-2-yl]methyl}-2,3,6,7- |
| | | tetrahydro-1H-purine-2,6-dione |
| | | ¹H NMR (400 MHz, CHLOROFORM-d) □ ppm 2.42 (s, 3 H), |
| | | 3.31 (br. s., 4 H), 3.34-3.38 (m, 3 H), 3.54 (s, 3 H), 3.72 (br. |
| | | s., 6 H), 4.03 (br. s., 2 H), 5.71 (s, 2 H), 7.35-7.43 (m, 2 H), |
| | | 7.76 (d, J = 3.95 Hz, 1 H), 7.80 (s, 1 H). |
| | | 481 |
| 57 | C | 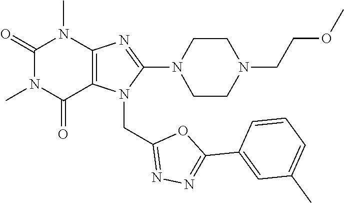 [SMILES: COCCN1CCN(c2nc3c(c(=O)n(C)c(=O)n3C)n2Cc2nnc(-c3cccc(C)c3)o2)CC1] |
| | | 8-[4-(2-methoxyethyl)piperazin-1-yl]-1,3-dimethyl-7-{[5-(3- |
| | | methylphenyl)-1,3,4-oxadiazol-2-yl]methyl}-2,3,6,7- |
| | | tetrahydro-1H-purine-2,6-dione |
| | | ¹H NMR (400 MHz, CHLOROFORM-d) d ppm 2.44 (s, 3 H), |
| | | 3.32 (d, J = 5.84 Hz, 4 H), 3.37 (s, 3 H), 3.38 (s, 3 H), 3.56 (s, |
| | | 3 H), 3.69 (br. s., 2 H), 3.77 (br. s., 2 H), 3.80 (br. s., 4 H), |
| | | 5.69 (s, 2 H), 7.34-7.45 (m, 2 H), 7.80 (d, J = 6.48 Hz, 1 H), |
| | | 7.84 (s, 1 H). |
| | | 495 |
| 58 | C | 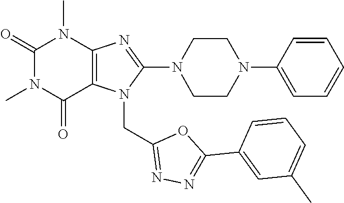 [SMILES: Cc1cccc(-c2nnc(Cn3c(N4CCN(c5ccccc5)CC4)nc4c3c(=O)n(C)c(=O)n4C)o2)c1] |
| | | 1,3-dimethyl-7-{[5-(3-methylphenyl)-1,3,4-oxadiazol-2- |
| | | yl]methyl}-8-(4-phenylpiperazin-1-yl)-2,3,6,7-tetrahydro-1H- |
| | | purine-2,6-dione |
| | | ¹H NMR (400 MHz, CHLOROFORM-d) d ppm 2.43 (s, 3 H), |
| | | 3.32 (br. s., 4 H), 3.39 (s, 3 H), 3.50 (br. s., 4 H), 3.58 (s, 3 |
| | | H), 5.71 (s, 2 H), 6.96 (br. s., 3 H), 7.28-7.44 (m, 4 H), 7.74- |
| | | 7.89 (m, 2 H). |
| | | 514 |
| 59 | C | 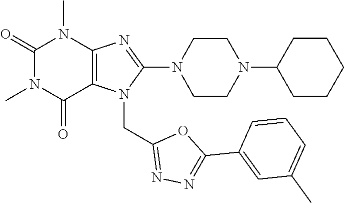 [SMILES: Cc1cccc(-c2nnc(Cn3c(N4CCN(C5CCCCC5)CC4)nc4c3c(=O)n(C)c(=O)n4C)o2)c1] |
| | | 8-(4-cyclohexylpiperazin-1-yl)-1,3-dimethyl-7-{[5-(3- |
| | | methylphenyl)-1,3,4-oxadiazol-2-yl]methyl}-2,3,6,7- |
| | | tetrahydro-1H-purine-2,6-dione |
| | | ¹H NMR (400 MHz, CHLOROFORM-d) d ppm 1.06-1.34 |
| | | (m, 5 H), 1.67-1.99 (m, 6 H), 2.43 (s, 3 H), 2.70-2.99 (m, |
| | | 4 H), 3.37 (s, 3 H), 3.38-3.55 (br. s., 4 H), 3.56 (s, 3 H), 5.66 |
| | | (s, 2 H), 7.33-7.44 (m, 2 H), 7.74-7.83 (m, 1 H), 7.83-7.88 |
| | | (m, 1 H) |
| | | 519 |
| 60 | D, B ⁽⁴⁾ | 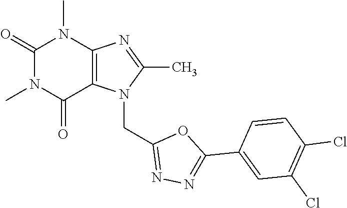 [SMILES: Cc1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazol-2-yl]methyl}- |
| | | 1,3,8-trimethyl-2,3,6,7-tetrahydro-1H-purine-2,6-dione |
| | | ¹H NMR (400 MHz, CHLOROFORM-d) δ ppm 2.61 (s, 3 H), |
| | | 3.43 (s, 3 H), 3.61 (s, 3 H), 5.89 (s, 2 H), 7.61 (d, J = 8.37 Hz, |
| | | 1 H), 7.86 (dd, J = 8.37, 2.05 Hz, 1 H), 8.12 (d, J = 1.90 Hz, 1 |
| | | H). ¹³C NMR (101 MHz, CHLOROFORM-d) δ ppm 13.3, |
| | | 27.9, 29.8, 39.3, 106.6, 122.8, 126.1, 128.8, 131.4, 133.8, |
| | | 135.4, 136.9, 148.2, 151.5, 155.1, 161.2, 164.2. |
| | | 421 |
| 61 | D, B ⁽⁴⁾ | 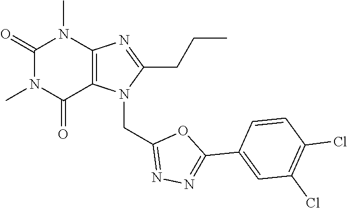 [SMILES: CCCc1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazol-2-yl]methyl}-1,3- |
| | | dimethyl-8-propyl-2,3,6,7-tetrahydro-1H-purine-2,6-dione |
| | | ¹H NMR (400 MHz, CHLOROFORM-d) δ ppm 0.98 (t, J = 7.45 |
| | | Hz, 3 H), 1.72-1.92 (m, 2 H), 2.77 (t, J = 7.58 Hz, 2 H), 3.36 |
| | | (s, 3 H), 3.55 (s, 3 H), 5.85 (s, 2 H), 7.54 (d, J = 8.34 Hz, 1 H), |
| | | 7.79 (dd, J = 8.34, 1.77 Hz, 1 H), 8.03 (d, J = 1.77 Hz, 1 H). 13C |
| | | NMR (101 MHz, CHLOROFORM-d) δ ppm 13.8, 21.1, 27.9, |
| | | 28.7, 29.8, 39.1, 106.4, 122.9, 126.1, 128.8, 131.4, 133.8, |
| | | 136.9, 148.4, 151.6, 155.1, 155.2, 161.5, 164.1. |
| | | 449 |
| 62 | D, B ⁽⁴⁾ | 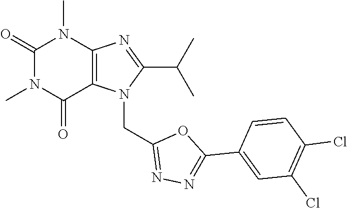 [SMILES: CC(C)c1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazol-2-yl]methyl}-1,3- |
| | | dimethyl-8-(propan-2-yl)-2,3,6,7-tetrahydro-1H-purine-2,6- |
| | | dione |
| | | ¹H NMR (400 MHz, CHLOROFORM-d) δ ppm 1.34 (d, |
| | | J = 6.57 Hz, 6 H), 3.10-3.26 (m, 1 H), 3.38 (s, 3 H), 3.54- |
| | | 3.63 (m, 3 H), 5.88 (s, 2 H), 7.57 (d, J = 8.34 Hz, 1 H), 7.76- |
| | | 7.90 (m, 1 H), 8.07 (s, 1 H). 13C NMR (101 MHz, |
| | | CHLOROFORM-d) δ ppm 21.4, 26.3, 27.9, 29.9, 38.9, |
| | | 106.1, 122.9, 126.0, 128.8, 131.3, 133.8, 136.8, 148.4, |
| | | 151.6, 155.3, 159.8, 161.7, 164.1. |
| | | 449 |
| 63 | D, B ⁽⁴⁾ | 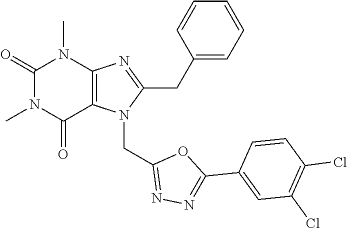 [SMILES: Cn1c(=O)c2c(nc(Cc3ccccc3)n2Cc2nnc(-c3ccc(Cl)c(Cl)c3)o2)n(C)c1=O] |
| | | 8-benzyl-7-{[5-(3,4-dichlorophenyl)-1,3,4-oxadiazol-2- |
| | | yl]methyl}-1,3-dimethyl-2,3,6,7-tetrahydro-1H-purine-2,6- |
| | | dione |
| | | ¹H NMR (400 MHz, CHLOROFORM-d) δ ppm 3.39 (s, 3 H), |
| | | 3.63 (s, 3 H), 4.28 (s, 2 H), 5.73 (s, 2 H), 7.10-7.22 (m, 3 |
| | | H), 7.22-7.29 (m, 2 H), 7.56 (d, J = 8.34 Hz, 1 H), 7.74 (dd, |
| | | J = 8.34, 2.02 Hz, 1 H), 7.97 (d, J = 2.02 Hz, 1 H). ¹³C NMR |
| | | (101 MHz, CHLOROFORM-d) δ ppm 28.0, 29.9, 33.6, 39.3, |
| | | 107.1, 122.8, 126.0, 127.5, 128.3, 128.7, 129.1, 131.3, |
| | | 133.7, 134.3, 136.7, 148.1, 151.5, 152.9, 155.2, 161.2, |
| | | 163.9. |
| | | 497 |

⁽¹⁾Example 1 was prepared by analogy with General Procedure C, with the exception that
methanol was used as the solvent and sodium methoxide was used to displace the
bromine.
⁽²⁾Example 23 was prepared from Example 6, which itself was prepared using General
Procedure C. Thiothers (0.08 mmol) were suspended in methanol (1 mL) and diluted with
water (1 mL). Oxone (0.16 mmol) was then added to the suspension and the reaction
mixture was stirred at rt overnight. 3 additional equivalents of oxone were then added and
the reaction was left for a further 24 hours. The mixture was then concentratred in vacuo
and products were isolated by preparative liquid chromatography.
⁽³⁾Example 24 was prepared from Example 7, which itself was prepared using General
Procedure G. The procedure used was identical to that used for Example 23.
⁽⁴⁾ General Procedure D was used to prepare an intermediate which was further reacted
under General Procedure B.

Further compounds of the examples and prodrugs thereof are described in Table 2 below. These compounds were prepared in accordance with the general procedures indicated or the specific procedures indicated below.

TABLE 2

| | | Structure |
|---|---|---|
| | General | Name |
| Example | Procedure | MS [M + H]⁺ |
| 64 | C′ | 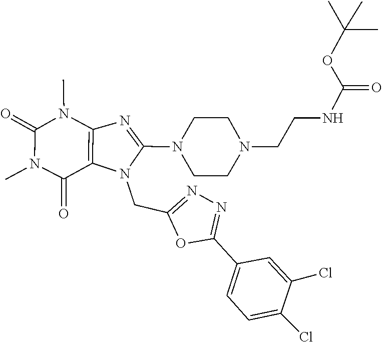 [SMILES: Cn1c(=O)c2c(nc(N3CCN(CCNC(=O)OC(C)(C)C)CC3)n2Cc2nnc(-c3ccc(Cl)c(Cl)c3)o2)n(C)c1=O] |
| | | tert-butyl N-{2-[4-(7-{[5-(3,4-dichlorophenyl)- |
| | | 1,3,4-oxadiazol-2-yl]methyl}-1,3-dimethyl-2,6- |
| | | dioxo-2,3,6,7-tetrahydro-1H-purin-8-yl)piperazin-1- |
| | | yl]ethyl}carbamate |
| | | m/z 634 |
| 65 | C′ | 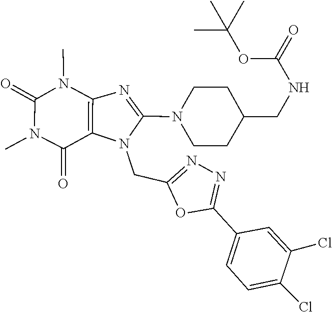 [SMILES: Cn1c(=O)c2c(nc(N3CCC(CNC(=O)OC(C)(C)C)CC3)n2Cc2nnc(-c3ccc(Cl)c(Cl)c3)o2)n(C)c1=O] |
| | | tert-butyl N-{[1-(7-{[5-(3,4-dichlorophenyl)-1,3,4- |
| | | oxadiazol-2-yl]methyl}-1,3-dimethyl-2,6-dioxo- |
| | | 2,3,6,7-tetrahydro-1H-purin-8-yl)piperidin-4- |
| | | yl]methyl}carbamate |
| | | m/z 619 |
| 66 | C′ | 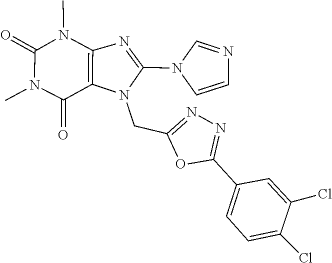 [SMILES: Cn1c(=O)c2c(nc(-n3ccnc3)n2Cc2nnc(-c3ccc(Cl)c(Cl)c3)o2)n(C)c1=O] |
| | | 7-{[5-(3,4-dichlorophenyl)-1,3,4-oxadiazol-2- |
| | | yl]methyl}-8-(1H-imidazol-1-yl)-1,3-dimethyl- |
| | | 2,3,6,7-tetrahydro-1H-purine-2,6-dione |
| | | m/z 473 |
| 67 | C′ | 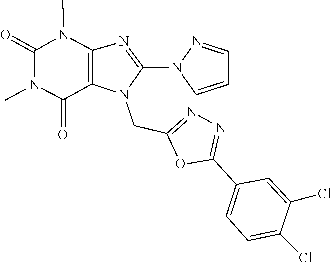 [SMILES: Cn1c(=O)c2c(nc(-n3cccn3)n2Cc2nnc(-c3ccc(Cl)c(Cl)c3)o2)n(C)c1=O] |
| | | 7-{[5-(3,4-dichlorophenyl)-1,3,4-oxadiazol-2- |
| | | yl]methyl}-1,3-dimethyl-8-(1H-pyrazol-1-yl)- |
| | | 2,3,6,7-tetrahydro-1H-purine-2,6-dione |
| | | m/z 473 |
| 68 | C′ | 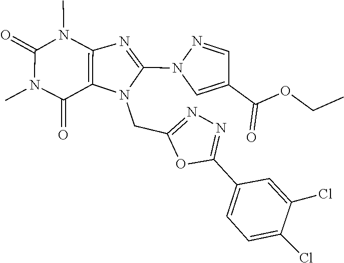 [SMILES: CCOC(=O)c1cnn(-c2nc3c(c(=O)n(C)c(=O)n3C)n2Cc2nnc(-c3ccc(Cl)c(Cl)c3)o2)c1] |
| | | ethyl 1-(7-{[5-(3,4-dichlorophenyl)-1,3,4- |
| | | oxadiazol-2-yl]methyl}-1,3-dimethyl-2,6-dioxo- |
| | | 2,3,6,7-tetrahydro-1H-purin-8-yl)-1H-pyrazole-4- |
| | | carboxylate |
| | | m/z 545 |
| 69 | B′ | 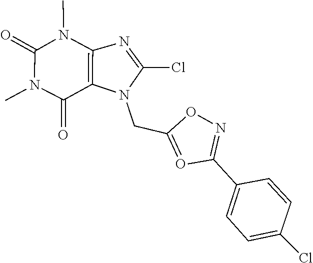 |
| | | 8-chloro-7-{[3-(4-chlorophenyl)-1,2,4-oxadiazol-5- |
| | | yl]methyl}-1,3-dimethyl-2,3,6,7-tetrahydro-1H- |
| | | purine-2,6-dione |
| | | m/z 407 |
| 70 | C′ | 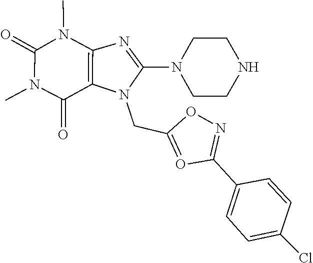 |
| | | 7-{[3-(4-chlorophenyl)-1,2,4-oxadiazol-5- |
| | | yl]methyl}-1,3-dimethyl-8-(piperazin-1-yl)-2,3,6,7- |
| | | tetrahydro-1H-purine-2,6-dione |
| | | m/z 457 |
| 71 | B′ | 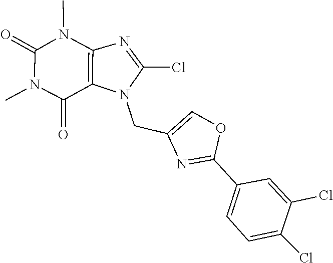 [SMILES: Cn1c(=O)c2c(nc(Cl)n2Cc2coc(-c3ccc(Cl)c(Cl)c3)n2)n(C)c1=O] |
| | | 8-chloro-7-{[2-(3,4-dichlorophenyl)-1,3-oxazol-4- |
| | | yl]methyl}-1,3-dimethyl-2,3,6,7-tetrahydro-1H- |
| | | purine-2,6-dione |
| | | m/z 440 |
| 72 | C′ | 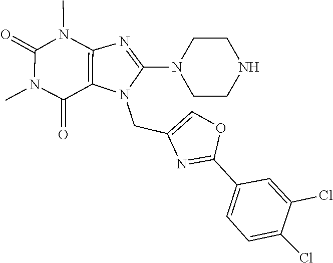 [SMILES: Cn1c(=O)c2c(nc(N3CCNCC3)n2Cc2coc(-c3ccc(Cl)c(Cl)c3)n2)n(C)c1=O] |
| | | 7-{[2-(3,4-dichlorophenyl)-1,3-oxazol-4-yl]methyl}- |
| | | 1,3-dimethyl-8-(piperazin-1-yl)-2,3,6,7-tetrahydro- |
| | | 1H-purine-2,6-dione |
| | | m/z 490 |
| 73 | B′ | 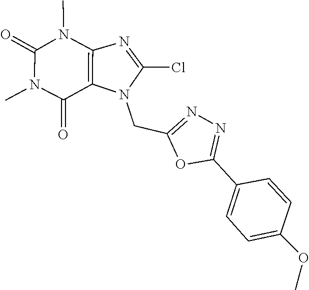 [SMILES: COc1ccc(-c2nnc(Cn3c(Cl)nc4c3c(=O)n(C)c(=O)n4C)o2)cc1] |
| | | 8-chloro-7-{[5-(4-methoxyphenyl)-1,3,4-oxadiazol-2- |
| | | yl]methyl}-1,3-dimethyl-2,3,6,7-tetrahydro-1H- |
| | | purine-2,6-dione |
| | | m/z 403 |
| 74 | C′ | 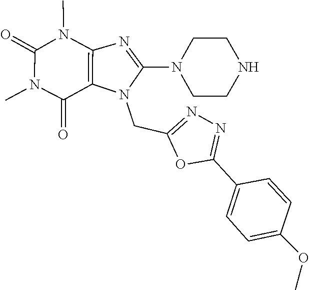 [SMILES: COc1ccc(-c2nnc(Cn3c(N4CCNCC4)nc4c3c(=O)n(C)c(=O)n4C)o2)cc1] |
| | | 7-{[5-(4-methoxyphenyl)-1,3,4-oxadiazol-2- |
| | | yl]methyl}-1,3-dimethyl-8-(piperazin-1-yl)-2,3,6,7- |
| | | tetrahydro-1H-purine-2,6-dione |
| | | m/z 453 |
| 75 | B′ | 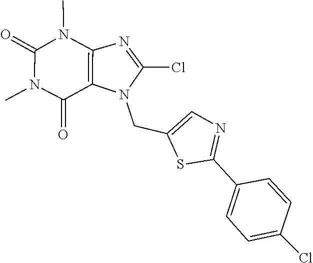 [SMILES: Cn1c(=O)c2c(nc(Cl)n2Cc2cnc(-c3ccc(Cl)cc3)s2)n(C)c1=O] |
| | | 8-chloro-7-{[2-(4-chlorophenyl)-1,3-thiazol-5- |
| | | yl]methyl}-1,3-dimethyl-2,3,6,7-tetrahydro-1H- |
| | | purine-2,6-dione |
| | | m/z 422 |
| 76 | C′ | 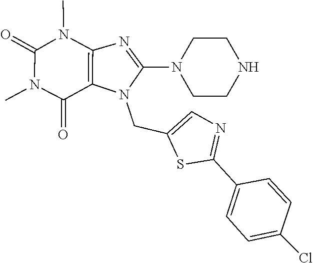 [SMILES: Cn1c(=O)c2c(nc(N3CCNCC3)n2Cc2cnc(-c3ccc(Cl)cc3)s2)n(C)c1=O] |
| | | 7-{[2-(4-chlorophenyl)-1,3-thiazol-5-yl]methyl}- |
| | | 1,3-dimethyl-8-(piperazin-1-yl)-2,3,6,7-tetrahydro- |
| | | 1H-purine-2,6-dione |
| | | m/z 472 |
| 77 | B′ | 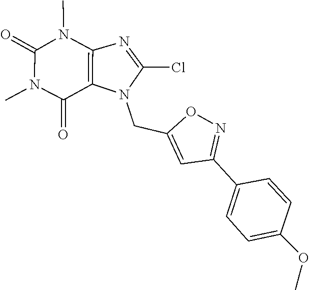 [SMILES: COc1ccc(-c2cc(Cn3c(Cl)nc4c3c(=O)n(C)c(=O)n4C)on2)cc1] |
| | | 8-chloro-7-{[3-(4-methoxyphenyl)-1,2-oxazol-5- |
| | | yl]methyl}-1,3-dimethyl-2,3,6,7-tetrahydro-1H- |
| | | purine-2,6-dione |
| | | m/z 402 |
| 78 | C′ | 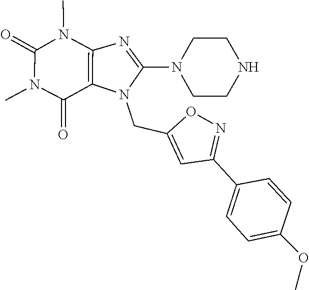 [SMILES: COc1ccc(-c2cc(Cn3c(N4CCNCC4)nc4c3c(=O)n(C)c(=O)n4C)on2)cc1] |
| | | 7-{[3-(4-methoxyphenyl)-1,2-oxazol-5-yl]methyl}- |
| | | 1,3-dimethyl-8-(piperazin-1-yl)-2,3,6,7-tetrahydro- |
| | | 1H-purine-2,6-dione |
| | | m/z 452 |
| 79 | C′ | 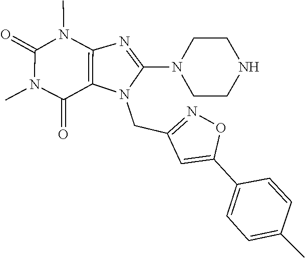 |
| | | 1,3-dimethyl-7-{[5-(4-methylphenyl)-1,2-oxazol-3- |
| | | yl]methyl}-8-(piperazin-1-yl)-2,3,6,7-tetrahydro- |
| | | 1H-purine-2,6-dione |
| | | m/z 436 |
| 80⁽¹⁾ | B′ | 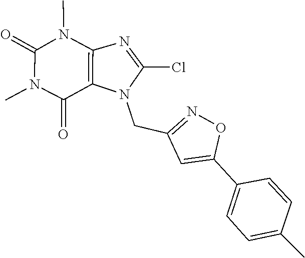 |
| | | 8-chloro-1,3-dimethyl-7-{[5-(4-methylphenyl)-1,2-oxazol- |
| | | 3-yl]methyl}-2,3,6,7-tetrahydro-1H-purine-2,6-dione |
| | | m/z 386 |
| 81 | C′ | 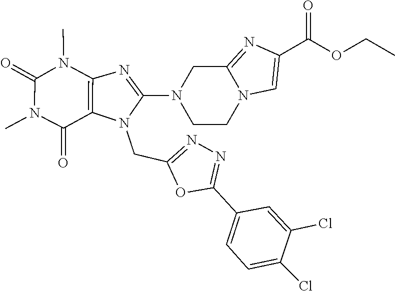 [SMILES: CCOC(=O)c1cn2c(n1)CN(c1nc3c(c(=O)n(C)c(=O)n3C)n1Cc1nnc(-c3ccc(Cl)c(Cl)c3)o1)CC2] |
| | | ethyl 7-(7-{[5-(3,4-dichlorophenyl)-1,3,4-oxadiazol-2- |
| | | yl]methyl}-1,3-dimethyl-2,6-dioxo-2,3,6,7-tetrahydro-1H- |
| | | purin-8-yl)-5H,6H,7H,8H-imidazo[1,2-a]pyrazine-2- |
| | | carboxylate |
| | | m/z 600 |
| 82 | C′ | 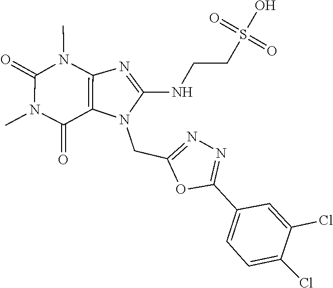 [SMILES: Cn1c(=O)c2c(nc(NCCS(=O)(=O)O)n2Cc2nnc(-c3ccc(Cl)c(Cl)c3)o2)n(C)c1=O] |
| | | 2-[(7-{[5-(3,4-dichlorophenyl)-1,3,4-oxadiazol-2- |
| | | yl]methyl}-1,3-dimethyl-2,6-dioxo-2,3,6,7-tetrahydro-1H- |
| | | purin-8-yl)amino]ethane-1-sulfonic acid |
| | | LCMS [M + H₃O]+ 548 |
| 83 | C′ | 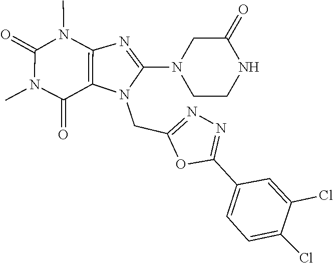 [SMILES: Cn1c(=O)c2c(nc(N3CCNC(=O)C3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}- |
| | | 1,3-dimethyl-8-(3-oxopiperazin-1-yl)-2,3,6,7-tetrahydro- |
| | | 1H-purine-2,6-dione |
| | | m/z 505 |
| 84 | C′ | 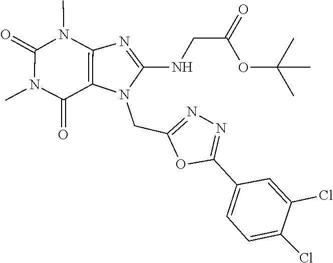 [SMILES: Cn1c(=O)c2c(nc(NCC(=O)OC(C)(C)C)n2Cc2nnc(-c3ccc(Cl)c(Cl)c3)o2)n(C)c1=O] |
| | | tert-butyl 2-[(7-{[5-(3,4-dichlorophenyl)-1,3,4-oxadiazol-2- |
| | | yl]methyl}-1,3-dimethyl-2,6-dioxo-2,3,6,7-tetrahydro-1H- |
| | | purin-8-yl)amino]acetate |
| | | m/z 536 |
| 85 | C′ | 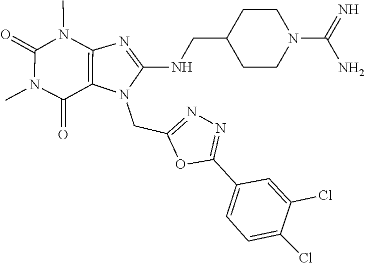 [SMILES: Cn1c(=O)c2c(nc(NCC3CCN(C(=N)N)CC3)n2Cc2nnc(-c3ccc(Cl)c(Cl)c3)o2)n(C)c1=O] |
| | | 4-{[(7-{[5-(3,4-dichlorophenyl)-1,3,4-oxadiazol-2- |
| | | yl]methyl}-1,3-dimethyl-2,6-dioxo-2,3,6,7-tetrahydro-1H- |
| | | purin-8-yl)amino]methyl}piperidine-1-carboximidamide |
| | | m/z 561 |
| 86 | C′ | 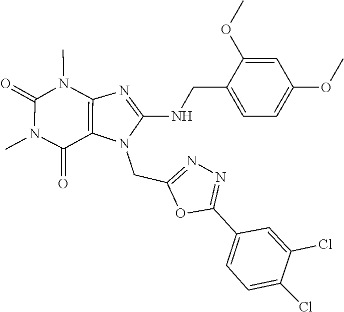 |
| | | 7-{[5-(3,4-dichlorophenyl)-1,3,4-oxadiazol-2-yl]methyl}-8- |
| | | {[(2,4-dimethoxyphenyl)methyl]amino}-1,3-dimethyl- |
| | | 2,3,6,7-tetrahydro-1H-purine-2,6-dione |
| | | m/z 572 |
| 87 | D′ | 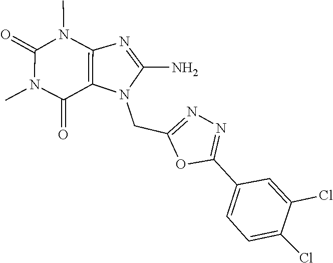 [SMILES: Cn1c(=O)c2c(nc(N)n2Cc2nnc(-c3ccc(Cl)c(Cl)c3)o2)n(C)c1=O] |
| | | 8-amino-7-{[5-(3,4-dichlorophenyl)-1,3,4-oxadiazol-2- |
| | | yl]methyl}-1,3-dimethyl-2,3,6,7-tetrahydro-1H-purine-2,6- |
| | | dione |
| | | m/z 422 |
| 88 | B′ | 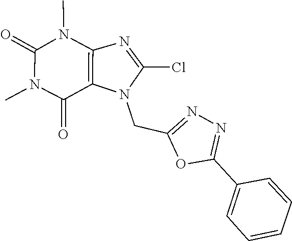 [SMILES: Cn1c(=O)c2c(nc(Cl)n2Cc2nnc(-c3ccccc3)o2)n(C)c1=O] |
| | | 8-chloro-1,3-dimethyl-7-[(5-phenyl-1,3,4-oxadiazol-2- |
| | | yl)methyl]-2,3,6,7-tetrahydro-1H-purine-2,6-dione |
| | | m/z 373 |
| 89 | B′ | 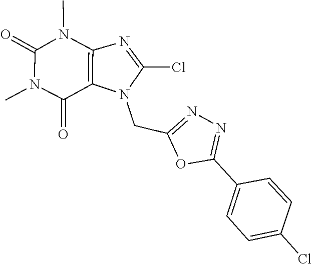 [SMILES: Cn1c(=O)c2c(nc(Cl)n2Cc2nnc(-c3ccc(Cl)cc3)o2)n(C)c1=O] |
| | | 8-chloro-7-{[5-(4-chlorophenyl)-1,3,4-oxadiazol-2- |
| | | yl]methyl}-1,3-dimethyl-2,3,6,7-tetrahydro-1H-purine-2,6- |
| | | dione |
| | | m/z 407 |
| 90 | B′ | 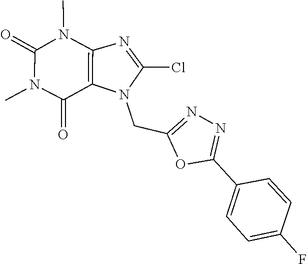 [SMILES: Cn1c(=O)c2c(nc(Cl)n2Cc2nnc(-c3ccc(F)cc3)o2)n(C)c1=O] |
| | | 8-chloro-7-{[5-(4-fluorophenyl)-1,3,4-oxadiazol-2- |
| | | yl]methyl}-1,3-dimethyl-2,3,6,7-tetrahydro-1H-purine-2,6- |
| | | dione |
| | | m/z 391 |
| 91 | B′ | 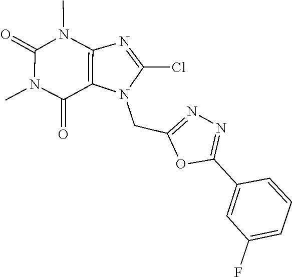 [SMILES: Cn1c(=O)c2c(nc(Cl)n2Cc2nnc(-c3cccc(F)c3)o2)n(C)c1=O] |
| | | 8-chloro-7-{[5-(3-fluorophenyl)-1,3,4-oxadiazol-2- |
| | | yl]methyl}-1,3-dimethyl-2,3,6,7-tetrahydro-1H-purine-2,6- |
| | | dione |
| | | m/z 391 |
| 92 | B′ | 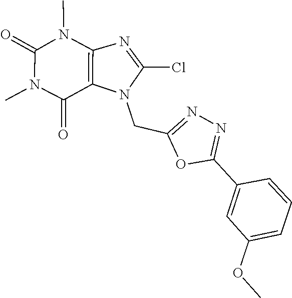 [SMILES: COc1cccc(-c2nnc(Cn3c(Cl)nc4c3c(=O)n(C)c(=O)n4C)o2)c1] |
| | | 8-chloro-7-{[5-(3-methoxyphenyl)-1,3,4-oxadiazol-2- |
| | | yl]methyl}-1,3-dimethyl-2,3,6,7-tetrahydro-1H-purine-2,6- |
| | | dione |
| | | m/z 403 |
| 93 | B′ | 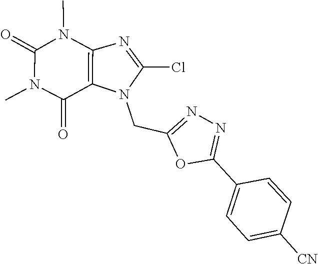 [SMILES: Cn1c(=O)c2c(nc(Cl)n2Cc2nnc(-c3ccc(C#N)cc3)o2)n(C)c1=O] |
| | | 4-{5-[(8-chloro-1,3-dimethyl-2,6-dioxo-2,3,6,7-tetrahydro- |
| | | 1H-purin-7-yl)methyl]-1,3,4-oxadiazol-2-yl}benzonitrile |
| | | m/z 398 |
| 94 | B′ | 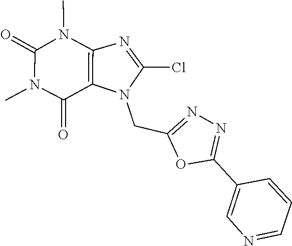 [SMILES: Cn1c(=O)c2c(nc(Cl)n2Cc2nnc(-c3cccnc3)o2)n(C)c1=O] |
| | | 8-chloro-1,3-dimethyl-7-{[5-(pyridin-3-yl)-1,3,4-oxadiazol- |
| | | 2-yl]methyl}-2,3,6,7-tetrahydro-1H-purine-2,6-dione |
| | | m/z 373 |
| 95 | B′ | 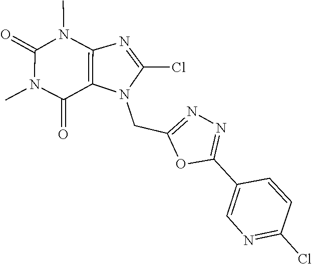 [SMILES: Cn1c(=O)c2c(nc(Cl)n2Cc2nnc(-c3ccc(Cl)nc3)o2)n(C)c1=O] |
| | | 8-chloro-7-{[5-(6-chloropyridin-3-yl)-1,3,4-oxadiazol-2- |
| | | yl]methyl}-1,3-dimethyl-2,3,6,7-tetrahydro-1H-purine-2,6- |
| | | dione |
| | | m/z 408 |
| 96 | B′ | 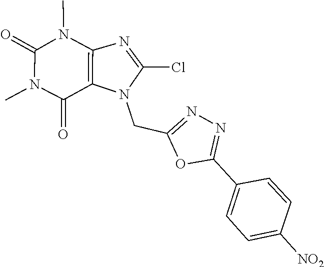 [SMILES: Cn1c(=O)c2c(nc(Cl)n2Cc2nnc(-c3ccc([N+](=O)[O-])cc3)o2)n(C)c1=O] |
| | | 8-chloro-1,3-dimethyl-7-{[5-(4-nitrophenyl)-1,3,4- |
| | | oxadiazol-2-yl]methyl}-2,3,6,7-tetrahydro-1H-purine-2,6- |
| | | dione |
| | | m/z 418 |
| 97 | B′ | 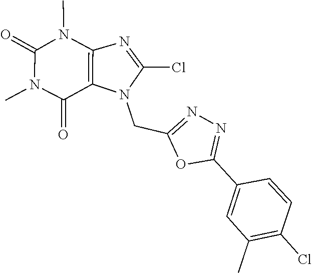 [SMILES: Cc1cc(-c2nnc(Cn3c(Cl)nc4c3c(=O)n(C)c(=O)n4C)o2)ccc1Cl] |
| | | 8-chloro-7-{[5-(4-chloro-3-methylphenyl)-1,3,4- |
| | | oxadiazol-2-yl]methyl}-1,3-dimethyl-2,3,6,7-tetrahydro- |
| | | 1H-purine-2,6-dione |
| | | m/z 421 |
| 98 | B′ | 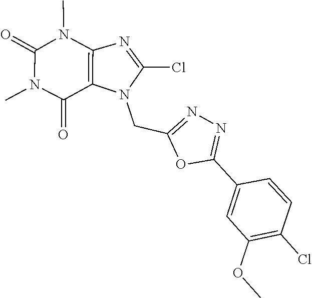 [SMILES: COc1cc(-c2nnc(Cn3c(Cl)nc4c3c(=O)n(C)c(=O)n4C)o2)ccc1Cl] |
| | | 8-chloro-7-{[5-(4-chloro-3-methoxyphenyl)-1,3,4- |
| | | oxadiazol-2-yl]methyl}-1,3-dimethyl-2,3,6,7-tetrahydro- |
| | | 1H-purine-2,6-dione |
| | | m/z 437 |
| 99 | B′ | 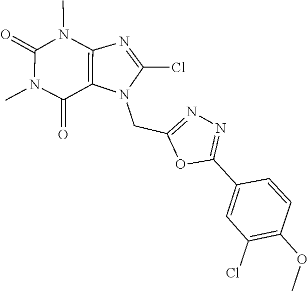 [SMILES: COc1ccc(-c2nnc(Cn3c(Cl)nc4c3c(=O)n(C)c(=O)n4C)o2)cc1Cl] |
| | | 8-chloro-7-{[5-(3-chloro-4-methoxyphenyl)-1,3,4- |
| | | oxadiazol-2-yl]methyl}-1,3-dimethyl-2,3,6,7-tetrahydro- |
| | | 1H-purine-2,6-dione |
| | | m/z 437 |
| 100 | | 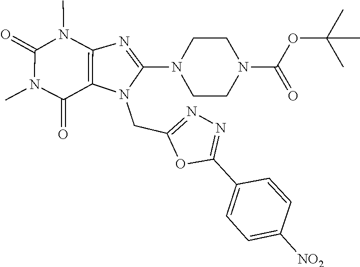 [SMILES: Cn1c(=O)c2c(nc(N3CCN(C(=O)OC(C)(C)C)CC3)n2Cc2nnc(-c3ccc([N+](=O)[O-])cc3)o2)n(C)c1=O] |
| | | tert-butyl 4-(1,3-dimethyl-7-{[5-(4-nitrophenyl)-1,3,4- |
| | | oxadiazol-2-yl]methyl}-2,6-dioxo-2,3,6,7-tetrahydro-1H- |
| | | purin-8-yl)piperazine-1-carboxylate |
| | | m/z 568 |
| 101 | B′ | 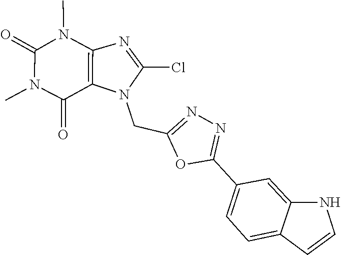 [SMILES: Cn1c(=O)c2c(nc(Cl)n2Cc2nnc(-c3ccc4cc[nH]c4c3)o2)n(C)c1=O] |
| | | 8-chloro-7-{[5-(1H-indol-6-yl)-1,3,4-oxadiazol-2- |
| | | yl]methyl}-1,3-dimethyl-2,3,6,7-tetrahydro-1H-purine-2,6- |
| | | dione |
| | | m/z 412 |
| 102 | B′ | 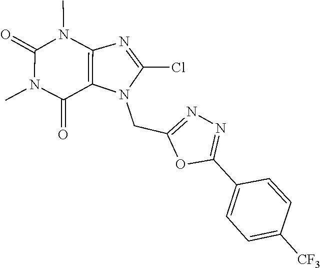 [SMILES: Cn1c(=O)c2c(nc(Cl)n2Cc2nnc(-c3ccc(C(F)(F)F)cc3)o2)n(C)c1=O] |
| | | 8-chloro-1,3-dimethyl-7-({5-[4-(trifluoromethyl)phenyl]- |
| | | 1,3,4-oxadiazol-2-yl}methyl)-2,3,6,7-tetrahydro-1H- |
| | | purine-2,6-dione |
| | | m/z 441 |
| 103 | B′ | 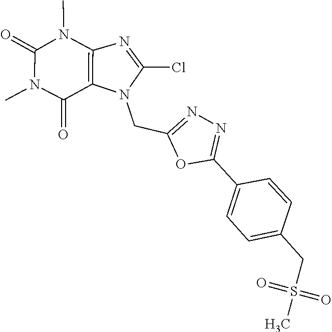 [SMILES: Cn1c(=O)c2c(nc(Cl)n2Cc2nnc(-c3ccc(CS(C)(=O)=O)cc3)o2)n(C)c1=O] |
| | | 8-chloro-7-({5-[4-(methanesulfonylmethyl)phenyl]-1,3,4- |
| | | oxadiazol-2-yl}methyl)-1,3-dimethyl-2,3,6,7-tetrahydro- |
| | | 1H-purine-2,6-dione |
| | | m/z 465 |
| 104 | B′ | 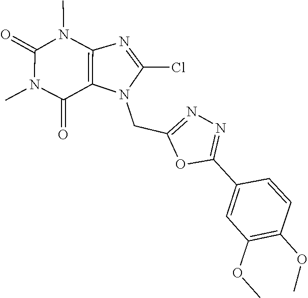 [SMILES: COc1ccc(-c2nnc(Cn3c(Cl)nc4c3c(=O)n(C)c(=O)n4C)o2)cc1OC] |
| | | 8-chloro-7-{[5-(3,4-dimethoxyphenyl)-1,3,4-oxadiazol-2- |
| | | yl]methyl}-1,3-dimethyl-2,3,6,7-tetrahydro-1H-purine-2,6- |
| | | dione |
| | | m/z 433 |
| 105 | | 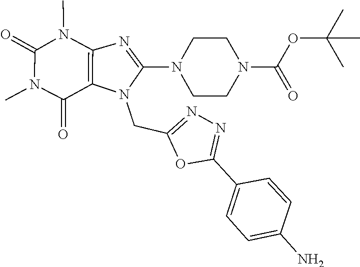 [SMILES: Cn1c(=O)c2c(nc(N3CCN(C(=O)OC(C)(C)C)CC3)n2Cc2nnc(-c3ccc(N)cc3)o2)n(C)c1=O] |
| | | tert-butyl 4-(7-{[5-(4-aminophenyl)-1,3,4-oxadiazol-2- |
| | | yl]methyl}-1,3-dimethyl-2,6-dioxo-2,3,6,7-tetrahydro-1H- |
| | | purin-8-yl)piperazine-1-carboxylate |
| | | m/z 538 |
| 106 | B′ | 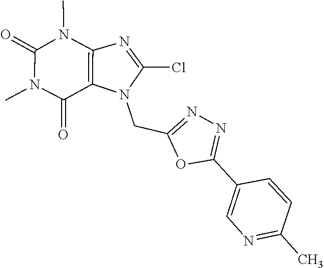 |
| | | 8-chloro-1,3-dimethyl-7-{[5-(6-methylpyridin-3-yl)-1,3,4- |
| | | oxadiazol-2-yl]methyl}-2,3,6,7-tetrahydro-1H-purine-2,6- |
| | | dione |
| | | m/z 388 |
| 107 | C′ | 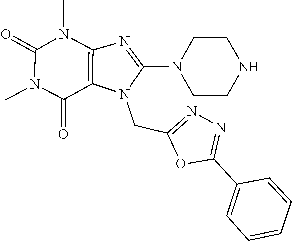 [SMILES: Cn1c(=O)c2c(nc(N3CCNCC3)n2Cc2nnc(-c3ccccc3)o2)n(C)c1=O] |
| | | 1,3-dimethyl-7-[(5-phenyl-1,3,4-oxadiazol-2-yl)methyl]-8- |
| | | (piperazin-1-yl)-2,3,6,7-tetrahydro-1H-purine-2,6-dione |
| | | m/z 423 |
| 108 | C′ | 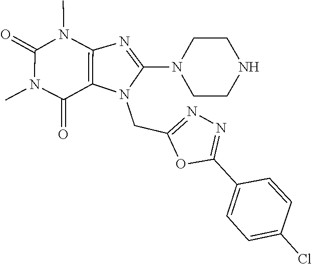 [SMILES: Cn1c(=O)c2c(nc(N3CCNCC3)n2Cc2nnc(-c3ccc(Cl)cc3)o2)n(C)c1=O] |
| | | 7-{[5-(4-chlorophenyl)-1,3,4-oxadiazol-2-yl]methyl}-1,3- |
| | | dimethyl-8-(piperazin-1-yl)-2,3,6,7-tetrahydro-1H-purine- |
| | | 2,6-dione |
| | | m/z 457 |
| 109 | C′ | 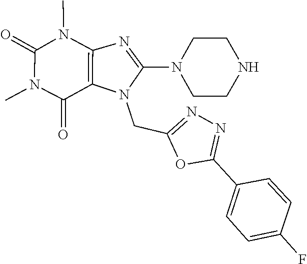 [SMILES: Cn1c(=O)c2c(nc(N3CCNCC3)n2Cc2nnc(-c3ccc(F)cc3)o2)n(C)c1=O] |
| | | 7-{[5-(4-fluorophenyl)-1,3,4-oxadiazol-2-yl]methyl}-1,3- |
| | | dimethyl-8-(piperazin-1-yl)-2,3,6,7-tetrahydro-1H-purine- |
| | | 2,6-dione |
| | | m/z 441 |
| 110 | C′ | 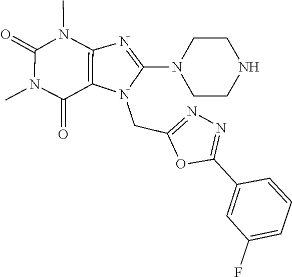 [SMILES: Cn1c(=O)c2c(nc(N3CCNCC3)n2Cc2nnc(-c3cccc(F)c3)o2)n(C)c1=O] |
| | | 7-{[5-(3-fluorophenyl)-1,3,4-oxadiazol-2-yl]methyl}-1,3- |
| | | dimethyl-8-(piperazin-1-yl)-2,3,6,7-tetrahydro-1H-purine- |
| | | 2,6-dione |
| | | m/z 441 |
| 111 | C′ | 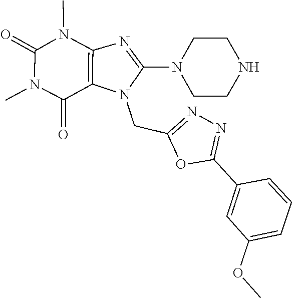 [SMILES: COc1cccc(-c2nnc(Cn3c(N4CCNCC4)nc4c3c(=O)n(C)c(=O)n4C)o2)c1] |
| | | 7-{[5-(3-methoxyphenyl)-1,3,4-oxadiazol-2-yl]methyl}- |
| | | 1,3-dimethyl-8-(piperazin-1-yl)-2,3,6,7-tetrahydro-1H- |
| | | purine-2,6-dione |
| | | m/z 453 |
| 112 | C′ | 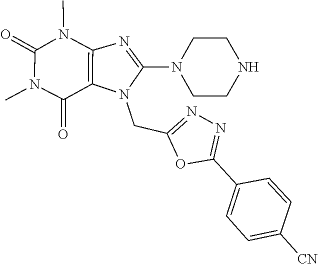 [SMILES: Cn1c(=O)c2c(nc(N3CCNCC3)n2Cc2nnc(-c3ccc(C#N)cc3)o2)n(C)c1=O] |
| | | 4-(5-{[1,3-dimethyl-2,6-dioxo-8-(piperazin-1-yl)-2,3,6,7- |
| | | tetrahydro-1H-purin-7-yl]methyl1-1,3,4-oxadiazol-2- |
| | | yl)benzonitrile |
| | | m/z 448 |
| 113 | C′ | 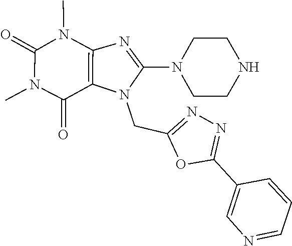 [SMILES: Cn1c(=O)c2c(nc(N3CCNCC3)n2Cc2nnc(-c3cccnc3)o2)n(C)c1=O] |
| | | 1,3-dimethyl-8-(piperazin-1-yl)-7-{[5-(pyridin-3-yl)-1,3,4- |
| | | oxadiazol-2-yl]methyl}-2,3,6,7-tetrahydro-1H-purine-2,6- |
| | | dione |
| | | m/z 424 |
| 114 | C′ | 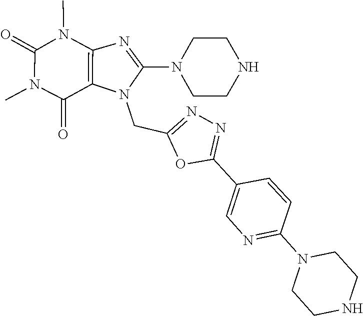 [SMILES: Cn1c(=O)c2c(nc(N3CCNCC3)n2Cc2nnc(-c3ccc(N4CCNCC4)nc3)o2)n(C)c1=O] |
| | | 1,3-dimethyl-8-(piperazin-1-yl)-7-({5-[6-(piperazin-1- |
| | | yl)pyridin-3-yl]-1,3,4-oxadiazol-2-yl}methyl)-2,3,6,7- |
| | | tetrahydro-1H-purine-2,6-dione |
| | | m/z 508 |
| 115 | C′ | 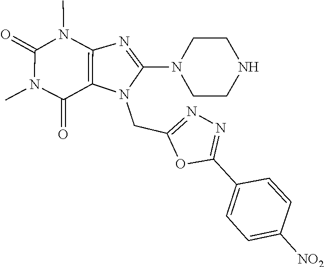 [SMILES: Cn1c(=O)c2c(nc(N3CCNCC3)n2Cc2nnc(-c3ccc([N+](=O)[O-])cc3)o2)n(C)c1=O] |
| | | 1,3-dimethyl-7-{[5-(4-nitrophenyl)-1,3,4-oxadiazol-2- |
| | | yl]methyl}-8-(piperazin-1-yl)-2,3,6,7-tetrahydro-1H- |
| | | purine-2,6-dione |
| | | m/z 468 |
| 116 | C′ | 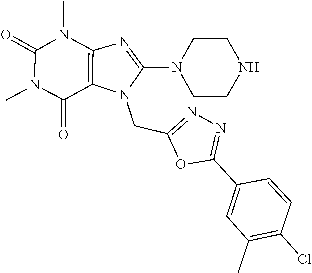 [SMILES: Cc1cc(-c2nnc(Cn3c(N4CCNCC4)nc4c3c(=O)n(C)c(=O)n4C)o2)ccc1Cl] |
| | | 7-{[5-(4-chloro-3-methylphenyl)-1,3,4-oxadiazol-2- |
| | | yl]methyl}-1,3-dimethyl-8-(piperazin-1-yl)-2,3,6,7- |
| | | tetrahydro-1H-purine-2,6-dione |
| | | m/z 471 |
| 117 | C′ | 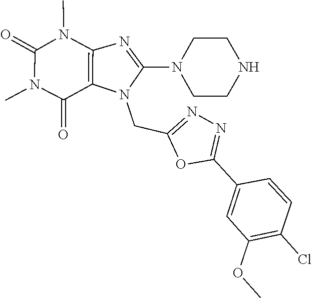 [SMILES: COc1cc(-c2nnc(Cn3c(N4CCNCC4)nc4c3c(=O)n(C)c(=O)n4C)o2)ccc1Cl] |
| | | 7-{[5-(4-chloro-3-methoxyphenyl)-1,3,4-oxadiazol-2- |
| | | yl]methyl}-1,3-dimethyl-8-(piperazin-1-yl)-2,3,6,7- |
| | | tetrahydro-1H-purine-2,6-dione |
| | | m/z 487 |
| 118 | C′ | 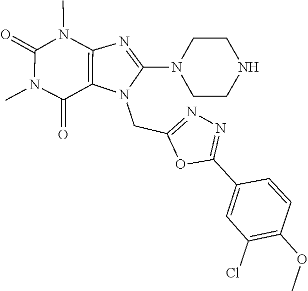 [SMILES: COc1ccc(-c2nnc(Cn3c(N4CCNCC4)nc4c3c(=O)n(C)c(=O)n4C)o2)cc1Cl] |
| | | 7-{[5-(3-chloro-4-methoxyphenyl)-1,3,4-oxadiazol-2- |
| | | yl]methyl}-1,3-dimethyl-8-(piperazin-1-yl)-2,3,6,7- |
| | | tetrahydro-1H-purine-2,6-dione |
| | | m/z 487 |
| 119 | C′ | 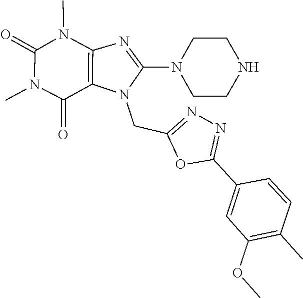 |
| | | 7-{[5-(3-methoxy-4-methylphenyl)-1,3,4-oxadiazol-2- |
| | | yl]methyl}-1,3-dimethyl-8-(piperazin-1-yl)-2,3,6,7- |
| | | tetrahydro-1H-purine-2,6-dione |
| | | m/z 467 |
| 120 | C′ | 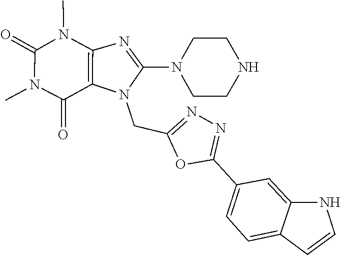 [SMILES: Cn1c(=O)c2c(nc(N3CCNCC3)n2Cc2nnc(-c3ccc4cc[nH]c4c3)o2)n(C)c1=O] |
| | | 7-{[5-(1H-indol-6-yl)-1,3,4-oxadiazol-2-yl]methyl}-1,3- |
| | | dimethyl-8-(piperazin-1-yl)-2,3,6,7-tetrahydro-1H-purine- |
| | | 2,6-dione |
| | | m/z 462 |
| 121 | C′ | 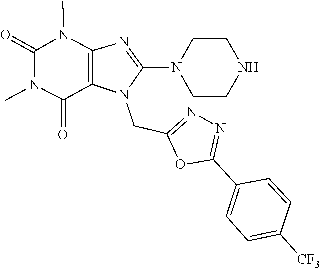 [SMILES: Cn1c(=O)c2c(nc(N3CCNCC3)n2Cc2nnc(-c3ccc(C(F)(F)F)cc3)o2)n(C)c1=O] |
| | | 1,3-dimethyl-8-(piperazin-1-yl)-7-({5-[4- |
| | | (trifluoromethyl)phenyl]-1,3,4-oxadiazol-2-yl}methyl)- |
| | | 2,3,6,7-tetrahydro-1H-purine-2,6-dione |
| | | m/z 491 |
| 122 | C′ | 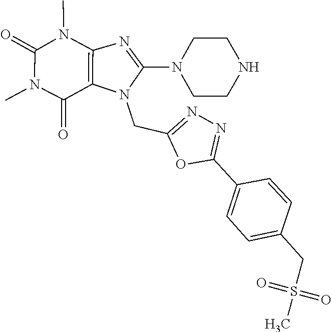 [SMILES: Cn1c(=O)c2c(nc(N3CCNCC3)n2Cc2nnc(-c3ccc(CS(C)(=O)=O)cc3)o2)n(C)c1=O] |
| | | 7-({5-[4-(methanesulfonylmethyl)phenyl]-1,3,4- |
| | | oxadiazol-2-yl}methyl)-1,3-dimethyl-8-(piperazin-1-yl)- |
| | | 2,3,6,7-tetrahydro-1H-purine-2,6-dione |
| | | m/z 515 |
| 123 | C′ | 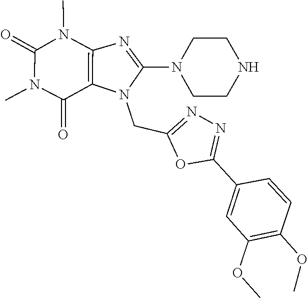 [SMILES: COc1ccc(-c2nnc(Cn3c(N4CCNCC4)nc4c3c(=O)n(C)c(=O)n4C)o2)cc1OC] |
| | | 7-{[5-(3,4-dimethoxyphenyl)-1,3,4-oxadiazol-2- |
| | | yl]methyl}-1,3-dimethyl-8-(piperazin-1-yl)-2,3,6,7- |
| | | tetrahydro-1H-purine-2,6-dione |
| | | m/z 483 |
| 124 | C′ | 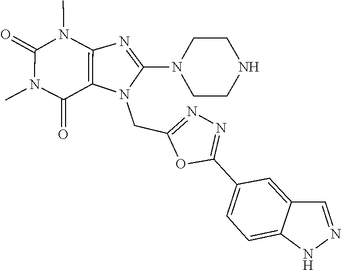 [SMILES: Cn1c(=O)c2c(nc(N3CCNCC3)n2Cc2nnc(-c3ccc4[nH]ncc4c3)o2)n(C)c1=O] |
| | | 7-{[5-(1H-indazol-5-yl)-1,3,4-oxadiazol-2-yl]methyl}-1,3- |
| | | dimethyl-8-(piperazin-1-yl)-2,3,6,7-tetrahydro-1H-purine- |
| | | 2,6-dione |
| | | m/z 463 |
| 125 | C′ | 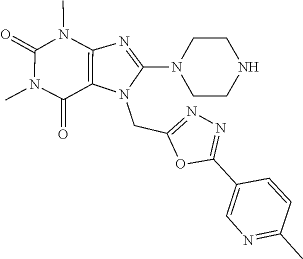 |
| | | 1,3-dimethyl-7-{[5-(6-methylpyridin-3-yl)-1,3,4-oxadiazol- |
| | | 2-yl]methyl}-8-(piperazin-1-yl)-2,3,6,7-tetrahydro-1H- |
| | | purine-2,6-dione |
| | | m/z 438 |
| 126 | C′ | 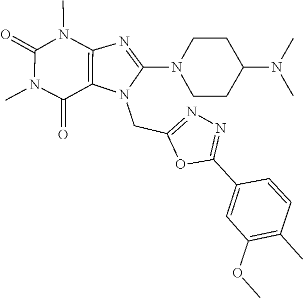 |
| | | 8-[4-(dimethylamino)piperidin-1-yl]-7-{[5-(3-methoxy-4- |
| | | methylphenyl)-1,3,4-oxadiazol-2-yl]methyl}-1,3-dimethyl- |
| | | 2,3,6,7-tetrahydro-1H-purine-2,6-dione |
| | | m/z 509 |
| 127 | C′ | 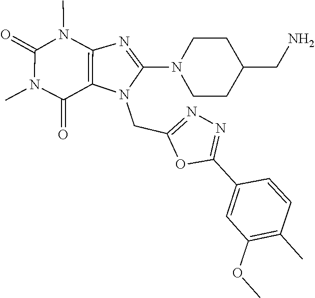 |
| | | 8-[4-(aminomethyl)piperidin-1-yl]-7-{[5-(3-methoxy-4- |
| | | methylphenyl)-1,3,4-oxadiazol-2-yl]methyl}-1,3-dimethyl- |
| | | 2,3,6,7-tetrahydro-1H-purine-2,6-dione |
| | | m/z 495 |
| 128 | C′ | 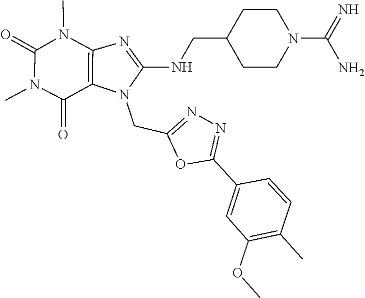 [SMILES: COc1cc(-c2nnc(Cn3c(NCC4CCN(C(=N)N)CC4)nc4c3c(=O)n(C)c(=O)n4C)o2)ccc1C] |
| | | 4-{[(7-{[5-(3-methoxy-4-methylphenyl)-1,3,4-oxadiazol-2- |
| | | yl]methyl}-1,3-dimethyl-2,6-dioxo-2,3,6,7-tetrahydro-1H- |
| | | purin-8-yl)amino]methyl}piperidine-1-carboximidamide |
| | | m/z 537 |
| 129 | C′ | 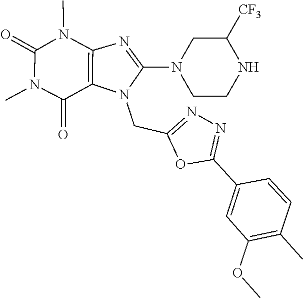 |
| | | 7-{[5-(3-methoxy-4-methylphenyl)-1,3,4-oxadiazol-2- |
| | | yl]methyl}-1,3-dimethyl-8-[3-(trifluoromethyl)piperazin-1- |
| | | yl]-2,3,6,7-tetrahydro-1H-purine-2,6-dione |
| | | m/z 535 |
| 130 | C′ | 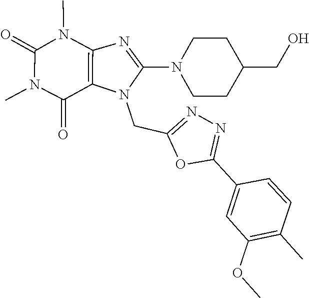 |
| | | 8-[4-(hydroxymethyl)piperidin-1-yl]-7-{[5-(3-methoxy-4- |
| | | methylphenyl)-1,3,4-oxadiazol-2-yl]methyl}-1,3-dimethyl- |
| | | 2,3,6,7-tetrahydro-1H-purine-2,6-dione |
| | | m/z 496 |
| 131 | C′ | 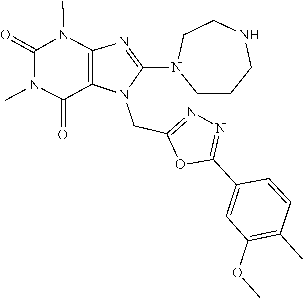 |
| | | 8-(1,4-diazepan-1-yl)-7-{[5-(3-methoxy-4-methylphenyl)- |
| | | 1,3,4-oxadiazol-2-yl]methyl}-1,3-dimethyl-2,3,6,7- |
| | | tetrahydro-1H-purine-2,6-dione |
| | | m/z 481 |
| 132 | C′ | 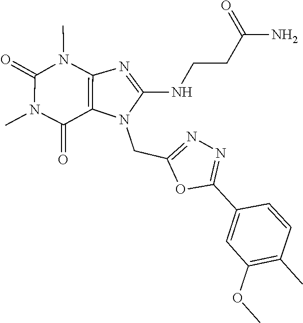 |
| | | 3-[(7-{[5-(3-methoxy-4-methylphenyl)-1,3,4-oxadiazol-2- |
| | | yl]methyl}-1,3-dimethyl-2,6-dioxo-2,3,6,7-tetrahydro-1H- |
| | | purin-8-yl)amino]propanamide |
| | | m/z 469 |
| 133 | C′ | 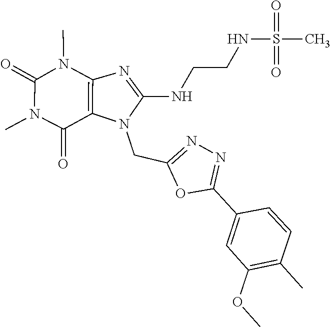 |
| | | N-{2-[(7-{[5-(3-methoxy-4-methylphenyl)-1,3,4- |
| | | oxadiazol-2-yl]methyl}-1,3-dimethyl-2,6-dioxo-2,3,6,7- |
| | | tetrahydro-1H-purin-8- |
| | | yl)amino]ethyl}methanesulfonamide |
| | | m/z 519 |
| 134 | C′ | 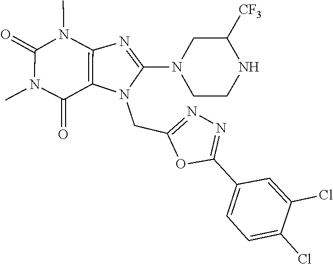 [SMILES: Cn1c(=O)c2c(nc(N3CCNC(C(F)(F)F)C3)n2Cc2nnc(-c3ccc(Cl)c(Cl)c3)o2)n(C)c1=O] |
| | | 7-{[5-(3,4-dichlorophenyl)-1,3,4-oxadiazol-2-yl]methyl}- |
| | | 1,3-dimethyl-8-[3-(trifluoromethyl)piperazin-1-yl]-2,3,6,7- |
| | | tetrahydro-1H-purine-2,6-dione |
| | | m/z 559 |
| 135 | C′ | 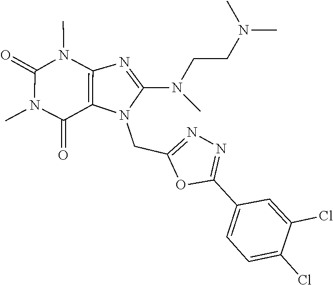 [SMILES: CN(C)CCN(C)c1nc2c(c(=O)n(C)c(=O)n2C)n1Cc1nnc(-c2ccc(Cl)c(Cl)c2)o1] |
| | | 7-{[5-(3,4-dichlorophenyl)-1,3,4-oxadiazol-2-yl]methyl}-8- |
| | | {[2-(dimethylamino)ethyl](methyl)amino}-1,3-dimethyl- |
| | | 2,3,6,7-tetrahydro-1H-purine-2,6-dione |
| | | m/z 507 |
| 136 | C′ | 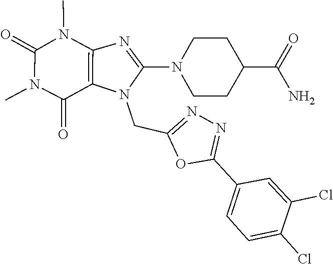 [SMILES: Cn1c(=O)c2c(nc(N3CCC(C(N)=O)CC3)n2Cc2nnc(-c3ccc(Cl)c(Cl)c3)o2)n(C)c1=O] |
| | | 1-(7-{[5-(3,4-dichlorophenyl)-1,3,4-oxadiazol-2- |
| | | yl]methyl}-1,3-dimethyl-2,6-dioxo-2,3,6,7-tetrahydro-1H- |
| | | purin-8-yl)piperidine-4-carboxamide |
| | | m/z 533 |
| 137 | C′ | 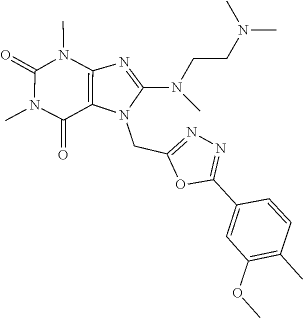 |
| | | 8-{[2-(dimethylamino)ethyl](methyl)amino}-7-{[5-(3- |
| | | methoxy-4-methylphenyl)-1,3,4-oxadiazol-2-yl]methyl}- |
| | | 1,3-dimethyl-2,3,6,7-tetrahydro-1H-purine-2,6-dione |
| | | m/z 483 |
| 138 | C′ | 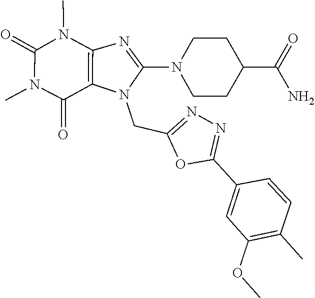 |
| | | 1-(7-{[5-(3-methoxy-4-methylphenyl)-1,3,4-oxadiazol-2- |
| | | yl]methyl}-1,3-dimethyl-2,6-dioxo-2,3,6,7-tetrahydro-1H- |
| | | purin-8-yl)piperidine-4-carboxamide |
| | | m/z 509 |
| 139 | C′ | 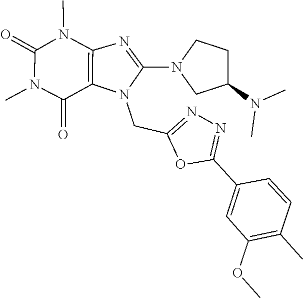 |
| | | 8-[(3R)-3-(dimethylamino)pyrrolidin-1-yl]-7-{[5-(3- |
| | | methoxy-4-methylphenyl)-1,3,4-oxadiazol-2-yl]methyl}- |
| | | 1,3-dimethyl-2,3,6,7-tetrahydro-1H-purine-2,6-dione |
| | | m/z 495 |
| 140 | C′ | 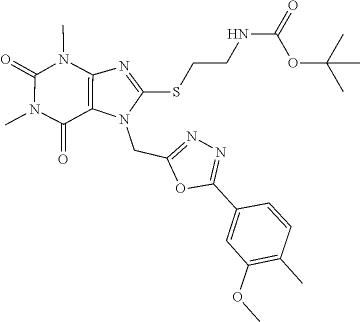 [SMILES: COc1cc(-c2nnc(Cn3c(SCCNC(=O)OC(C)(C)C)nc4c3c(=O)n(C)c(=O)n4C)o2)ccc1C] |
| | | tert-butyl N-{2-[(7-{[5-(3-methoxy-4-methylphenyl)-1,3,4- |
| | | oxadiazol-2-yl]methyl}-1,3-dimethyl-2,6-dioxo-2,3,6,7- |
| | | tetrahydro-1H-purin-8-yl)sulfanyl]ethyl}carbamate |
| | | m/z 558 |
| 141 | C′ | 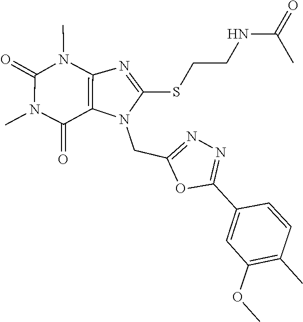 |
| | | N-{2-[(7-{[5-(3-methoxy-4-methylphenyl)-1,3,4- |
| | | oxadiazol-2-yl]methyl}-1,3-dimethyl-2,6-dioxo-2,3,6,7- |
| | | tetrahydro-1H-purin-8-yl)sulfanyl]ethyl}acetamide |
| | | m/z 500 |
| 142 | D′ | 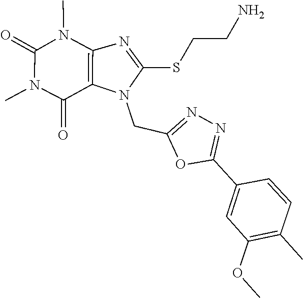 |
| | | 8-[(2-aminoethyl)sulfanyl]-7-{[5-(3-methoxy-4- |
| | | methylphenyl)-1,3,4-oxadiazol-2-yl]methyl}-1,3-dimethyl- |
| | | 2,3,6,7-tetrahydro-1H-purine-2,6-dione |
| | | m/z 458 |
| 143 | B′ | 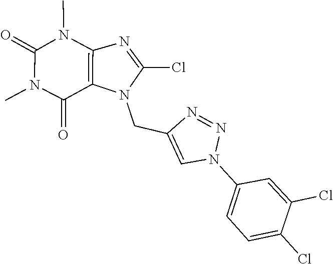 [SMILES: Cn1c(=O)c2c(nc(Cl)n2Cc2cn(-c3ccc(Cl)c(Cl)c3)nn2)n(C)c1=O] |
| | | 8-chloro-7-{[1-(3,4-dichlorophenyl)-1H-1,2,3-triazol-4- |
| | | yl]methyl}-1,3-dimethyl-2,3,6,7-tetrahydro-1H-purine-2,6- |
| | | dione |
| | | m/z 440 |
| 144 | C′ | 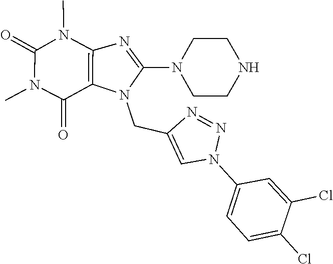 [SMILES: Cn1c(=O)c2c(nc(N3CCNCC3)n2Cc2cn(-c3ccc(Cl)c(Cl)c3)nn2)n(C)c1=O] |
| | | 7-{[1-(3,4-dichlorophenyl)-1H-1,2,3-triazol-4-yl]methyl}- |
| | | 1,3-dimethyl-8-(piperazin-1-yl)-2,3,6,7-tetrahydro-1H- |
| | | purine-2,6-dione |
| | | m/z 490 |

⁽¹⁾For Example 80, a mesylate leaving group was employed in place of the chlorine atom in the R⁴ substituted intermediates. These reactions were done under analogous conditions.

### Synthesis of Example 100

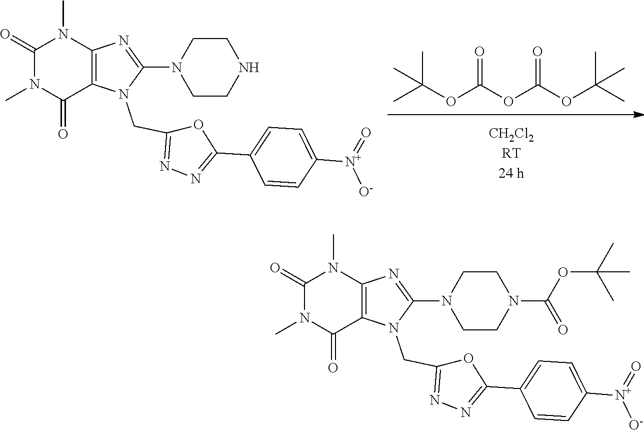

Di-tert-butyl dicarbonate (1.1 equiv.) was added to the compound of Example 115 (1.0 equiv.) dissolved in CH₂Cl₂ (0.1 mol/L) and the resulting mixture was stirred at room temperature. Upon completion the reaction mixture was poured into saturated sodium bicarbonate solution and extracted with CH₂Cl₂×3. The combined extracts were dried, concentrated, and purified by silica gel chromatography in a mixture of iso-hexane and ethyl acetate.

### Synthesis of Example 105

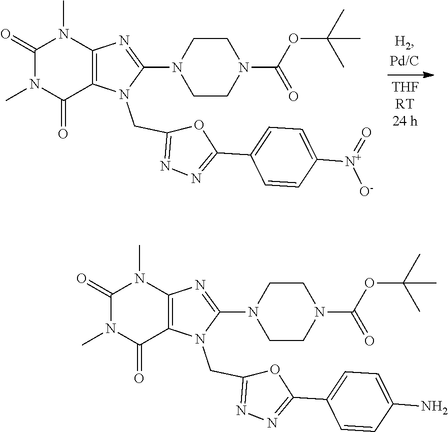 [SMILES: Cn1c(=O)c2c(nc(N3CCN(C(=O)OC(C)(C)C)CC3)n2Cc2nnc(-c3ccc([N+](=O)[O-])cc3)o2)n(C)c1=O ; Cn1c(=O)c2c(nc(N3CCN(C(=O)OC(C)(C)C)CC3)n2Cc2nnc(-c3ccc(N)cc3)o2)n(C)c1=O]

A mixture of the compound of Example 100 (1.0 equiv.) and Pd/C (0.05 equiv.) was stirred in tetrahydrofuran at room temperature for 24 h under hydrogen atmosphere provided by a balloon. Upon completion, Pd/C was removed by suction filtration and the reaction mixture was concentrated and purified by silica gel chromatography in a mixture of iso-hexane and ethyl acetate.

### Biological Assays

The biological activity of example compounds as described herein above was assessed using the following biological assays.

### Biological Assay 1: NUDT5 Inhibitor Activity by Malachite Green Reporter Assay

NUDT5 inhibitors were evaluated using a coupled enzymatic assay with detection of inorganic phosphate (Pi) using the malachite green assay. Following enzymatic hydrolysis of ADP-Ribose (Sigma-Aldrich A0572) by NUDT5, to yield AMP and ribose-5-phosphate, the latter product is continuously processed by a significant excess of alkaline phosphatase (Sigma-Aldrich, P0114). Formation of Pi is quantified based on the green complex (that absorbs at 630 nm) formed between malachite green and molybdate according to published procedure (Baykov, A. A., Evtushenko, O. A. and Avaeva, S. M., Anal Biochem, 171, 266-270 (1988)). Assay buffer consisted of 10 mM Tris-acetate at pH 8.0, 40 mM sodium chloride, 10 mM magnesium acetate, 0.005% Tween-20 and 1 mM dithiothreitol (DTT). The final conditions consisted of 6 nM recombinant human NUDT5, 50 μM ADP-Ribose and 10 U/ml of calf intestine alkaline phosphatase. The plates with compounds, enzymes and substrate were incubated at room temperature for 15 minutes, after which the reaction was terminated and the signal developed by the addition of the malachite green reagent using a MultiDrop (Thermo Scientific). Following vigorous shaking on plate shakers for a minimum of eight minutes the absorbance was read in a microplate reader (HidexSense) using a filter at 630 nm and a read time of 0.1 s per well. Inhibitors were used in dilutions from 100 μM to 1.7 nM in 3-fold dilution steps. Experiments were run in duplicate and confirmed in two independent experiments. IC₅₀ values were determined by curve fitting using ExcelFit.

Results obtained for the example compounds as described in Table 1 are provided in Table 3 below, and results obtained for the example compounds as described in Table 2 are provided in Table 4 below.

TABLE 3

| | Malachite |
|---|---|
| | Green |
| Ex. | Assay [nM] |
| 1 | 29.1 |
| 2 | 23.7 |
| 3 | 111 |
| 4 | >1000 |
| 5 | 12.5 |
| 6 | 36.9 |
| 7 | 65.9 |
| 8 | 162 |
| 9 | 15.0 |
| 10 | 22.9 |
| 11 | 84.6 |
| 12 | 42.5 |
| 13 | 759 |
| 14 | 164 |
| 15 | 432 |
| 16 | 53.8 |
| 17 | 39.0 |
| 18 | 151 |
| 19 | 29.1 |
| 20 | 30 |
| 21 | 50.3 |
| 22 | 35.1 |
| 23 | 24.4 |
| 24 | 22.7 |
| 25 | 30 |
| 26 | 324 |
| 27 | 19.3 |
| 28 | 37 |
| 29 | 43.8 |
| 30 | 33.2 |
| 31 | 105.8 |
| 32 | 76.7 |
| 33 | 17.8 |
| 34 | 24.7 |
| 35 | 34.8 |
| 36 | 34.0 |
| 37 | 19.8 |
| 38 | 18.5 |
| 39 | 32.7 |
| 40 | 20.3 |
| 41 | 24.2 |
| 42 | 26.4 |
| 43 | 26.8 |
| 44 | 21.5 |
| 45 | 14.9 |
| 46 | 12.0 |
| 47 | 11.9 |
| 48 | 69.4 |
| 49 | 356 |
| 50 | 144 |
| 51 | 31.3 |
| 52 | 378 |
| 53 | 806 |
| 54 | >1000 |
| 55 | 221 |
| 56 | 922 |
| 57 | >1000 |
| 58 | >1000 |
| 59 | >1000 |
| 60 | 28.4 |
| 61 | 246 |
| 62 | >1000 |
| 63 | >1000 |

TABLE 4

| | Malachite |
|---|---|
| | Green |
| | Assay |
| Ex. | [nM] |
| 64 | 226 |
| 65 | 33.8 |
| 66 | 58.2 |
| 67 | 200 |
| 68 | 214 |
| 69 | 296 |
| 70 | 57.4 |
| 71 | 81.6 |
| 72 | 21.0 |
| 73 | 1260 |
| 74 | 198 |
| 75 | 239 |
| 76 | 376 |
| 77 | 19700 |
| 78 | 491 |
| 79 | 530 |
| 80 | 3791 |
| 81 | 149 |
| 82 | 454 |
| 83 | 20.2 |
| 84 | 93.6 |
| 85 | 19.5 |
| 86 | 184 |
| 87 | 16.5 |
| 88 | 3588 |
| 89 | 620 |
| 90 | 4385 |
| 91 | 2146 |
| 92 | 596 |
| 93 | 918 |
| 94 | 6907 |
| 95 | 1380 |
| 96 | 1505 |
| 97 | 64.8 |
| 98 | 378 |
| 99 | 73.0 |
| 100 | 9436 |
| 101 | 358 |
| 102 | 484 |
| 103 | 61612 |
| 104 | >100000 |
| 105 | 24510 |
| 106 | 2920 |
| 107 | 212 |
| 108 | 99 |
| 109 | 345 |
| 110 | 129 |
| 111 | 119 |
| 112 | 255 |
| 113 | 1694 |
| 114 | 28984 |
| 115 | 286 |
| 116 | 21.6 |
| 117 | 64.2 |
| 118 | 17.4 |
| 119 | 35.7 |
| 120 | 80.5 |
| 121 | 51.6 |
| 122 | 24383 |
| 123 | 169 |
| 124 | 309 |
| 125 | 216 |
| 126 | 39.1 |
| 127 | 32.9 |
| 128 | 128 |
| 129 | 911 |
| 130 | 205 |
| 131 | 60.7 |
| 132 | 157 |
| 133 | 352 |
| 134 | 140 |
| 135 | 96.3 |
| 136 | 8.27 |
| 137 | 24.1 |
| 138 | 33.4 |
| 139 | 265 |
| 140 | 262 |
| 141 | 306 |
| 142 | 49.2 |
| 143 | 14000 |
| 144 | 155 |

### Biological Assay 2: Measurement of Target Engagement in Cells

Compounds showing potent activity by malachite green assay were then screened for NUDT5 engagement in cell lysates by thermal shift assay. Target engagement in cells was determined by the cellular thermal shift assay (CETSA), as described previously (Jafari, R., et al., Nature Protocols, 9, 2100-2122 (2014)).

Briefly, cell lysate thermal shift assays were performed with 1×10⁶ HL-60 cells per temperature/condition. Cells were collected and washed once with PBS and resuspended in 1× Tris-buffered saline (TBS) with protease inhibitor cocktail (Mini cOmplete, EDTA-free, Roche) at 60 μL/1×10⁶ cells. The cells were then aliquoted and lysed by freeze-thawing three times with three-minute incubations (3×, 3 min.+3 min.) using an ethanol/dry ice bath and water bath at 37° C. The lysates were then centrifuged at 20 000×g for 20 minutes at 4° C. to remove cellular debris. Supernatants were then transferred to PCR strip tubes and treated with 0.5 μL DMSO (0.8% v/v final) or 20 μM inhibitor for 20 minutes at room temperature. Lysates with compounds were heated at the indicated temperature in a Veriti Thermal Cycler (Applied Biosystems) for 3 minutes, then another 3 minutes at room temperature. They were then centrifuged at 20 000×g for 20 minutes at 4° C. to pellet protein aggregates and 45 μL was removed and prepared for western blotting analysis.

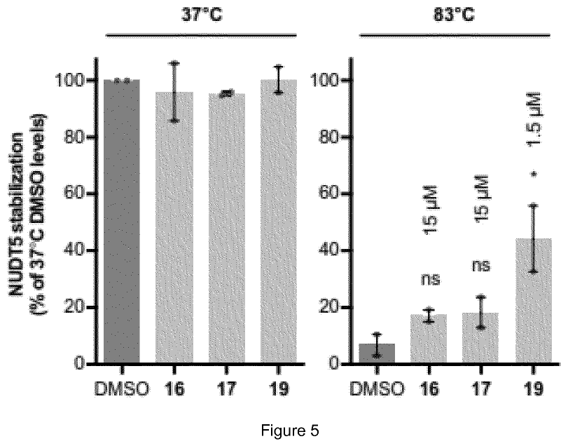

For CETSA experiments with cells treated in culture, 1×10⁶ HL-60 were treated with DMSO (0.2% v/v final) or 20 μM compound for 3 hours at 37° C. and 5% C₀₂ in a humidified incubator. T47D^{WT} cells were treated instead with 15 μM Compounds 16/17, and 1.5 μM Compound 19. The results are shown in FIG. 5. The cells were harvested, washed twice with PBS and resuspended in TBS with protease inhibitors, as above. Heating was identical as before, except that immediately following heating, the cell suspensions were snap frozen in a dry ice/ethanol bath and freeze-thawed as previously to lyse cells. Centrifugation at 20 000×g for 20 minutes at 4° C. removed cellular debris and protein aggregates, and lysates were prepared for western blotting.

Treatments, heating and lysis procedures for ITDRF_{CETSA} were performed identically to those for CETSA experiments, except NUDT5 inhibitors were added to cells at serial dilutions from 20 to 0.08 μM or 10 to 0.04 μM as indicated.

Gel electrophoresis and western blotting were performed as before. Primary antibodies were incubated overnight at 4° C. in 1:1 Li-Cor Blocking Buffer/TBS+0.05% Tween-20 (TBS-T) at the following concentrations: anti-NUDT5 (EZBiolabs+lab-purified, 1:1 000), or SOD1 (Santa Cruz, 1:1 000). Secondary antibodies were diluted at 1:15 000 in 1:1 Li-Cor blocking buffer/TBS-T and incubated for 1 hour at room temperature. Bands were visualized with an Odyssey Fc Imager and analyzed with Image Studio Software (Li-Cor Biosciences).

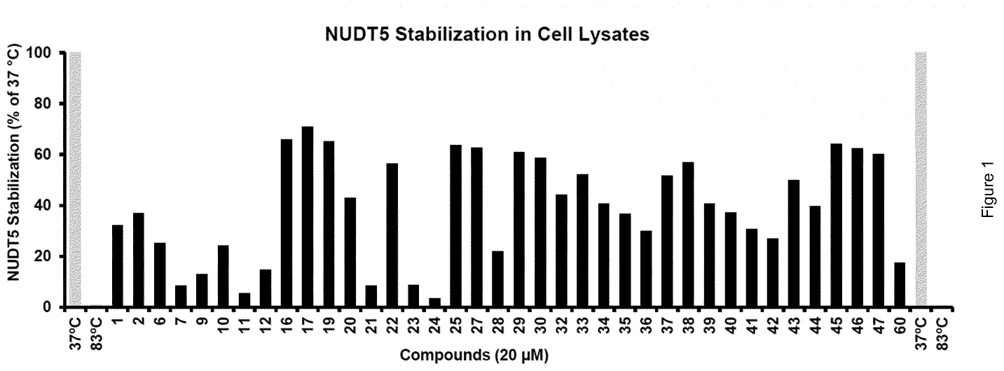

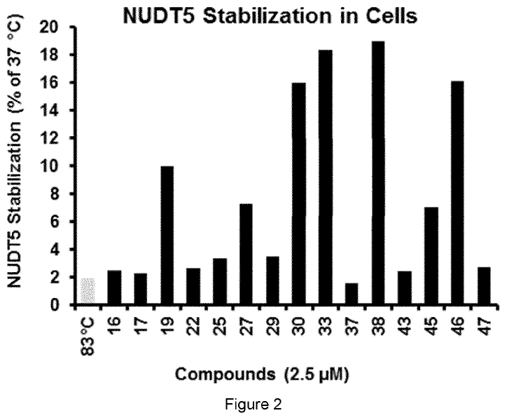

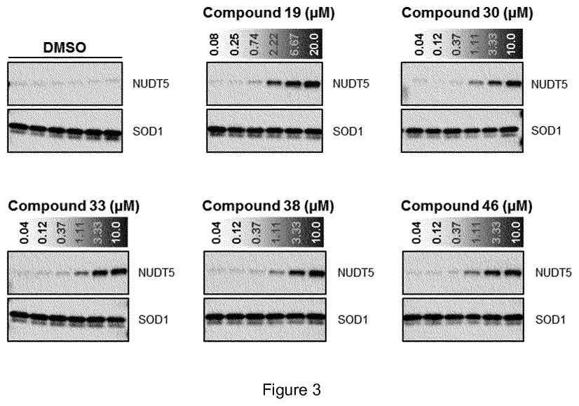

The results of these experiments are shown in the figures. FIG. 1 shows NUDT5 stabilization by inhibitors when added to HL-60 cell lysates. FIG. 2 shows NUDT5 stabilization by inhibitors added to HL-60 cells in culture. FIG. 3 shows the isothermal dose response fingerprint for certain inhibitors in HL-60 cells. FIG. 5 shows NUDT5 stabilization by inhibitors added to T47D cells in culture.

### Biological Assay 3: Assessment of the Utility of NUDT5 Inhibitors in Breast Cancer

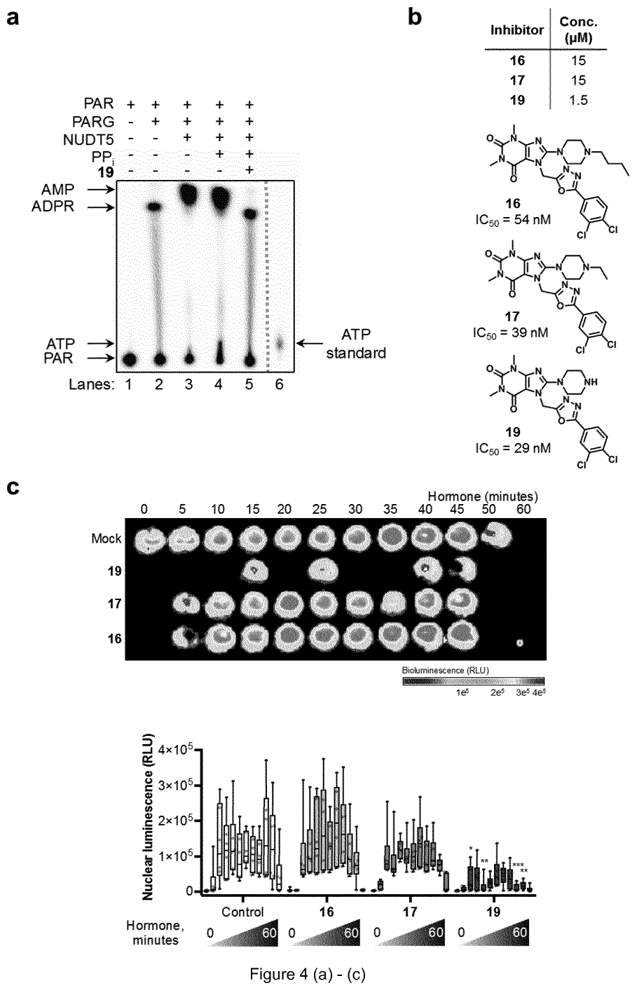

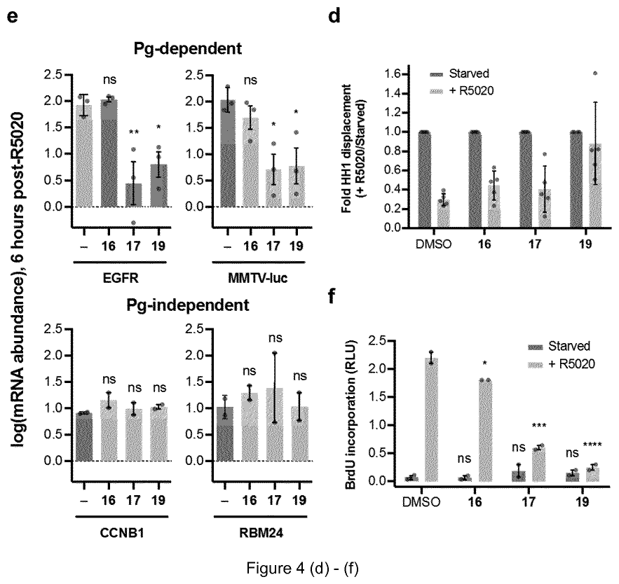

Thin layer chromatography (TLC) of products formed following processing of ³²P-PAR by purified PARG and NUDT5 in the absence or presence of Compound 19 and PPᵢ in vitro, was performed (Wright, R. H. G. et al., Science, 352, 1221-1225 (2016)). The results obtained are shown in FIG. 4a.

Certain example compounds were used at varying concentrations to determine their effect on hormone signaling and breast cancer with T47D breast adenocarcinoma cells (Compound 19, was used at 1.5 μM whereas the less potent derivatives Compound 16 and Compound 17 were used at 15 μM). The results obtained are shown in FIG. 4b and the selected concentrations were used to generate data obtained in FIGS. 4c, 4d, 4e and 4f.

In HR+ breast cancer cells, NUDT5 activity is required to produce ATP in the nucleus following hormone stimulation (Wright, R. H. G. et al., Science, 352, 1221-1225 (2016)). A nuclear-targeted luciferase reporter system was employed to monitor ATP production after hormone stimulation. The results obtained are shown in FIG. 4c.

Hormone-starved T47D^{WT} cells were treated with example compounds as described herein for two hours and then stimulated with the progesterone receptor agonist, R5020, for the time points indicated. Strong luminescence was seen from cells treated with Compound 16, Compound 17 or DMSO, the Compound 19-treated cells had reduced nuclear luminescence, indicating impaired nuclear ATP production. Quantification of the results obtained are shown in FIG. 4c.

Nuclear ATP is used by ATP-dependent chromatin remodeling enzymes, which disrupt the interactions of histones and DNA (Wright, R. H. G. et al., Science, 352, 1221-1225 (2016)). In conjunction with histone acetyltransferase and deacetylase complexes, this process regulates the transcriptional activation of genes (Vicent, G. P. et al., Genes Dev, 25, 845-862 (2011), Narlikar, G. J., Sundaramoorthy, R. and Owen-Hughes, T., Cell, 154, 490-503 (2013), Mayes, K. Qiu, Z., Alhazmi, A. and Landry, J. W., Adv Cancer Res, 121, 183-233 (2014)). Blocking nuclear ATP production prevents the activation of ATP-dependent chromatin remodeling enzymes and displacement of histones H1 and H2A/H2B (Wright, R. H. G. et al., Science, 352, 1221-1225 (2016)). Histone displacement following a thirty-minute R5020 exposure in T47D^{WT} cells treated with example compounds for two hours was analyzed. The results obtained are shown in FIG. 4d.

Without chromatin remodeling, the regulation of hormone-dependent genes and resultant cell proliferation should also be impaired (Wright, R. H. G. et al., Science, 352, 1221-1225 (2016)). Using T47D cells harboring an integrated mouse mammary tumor virus luciferase reporter (MMTV-luc; T47D^{M} cells), mRNA expression for progesterone-dependent genes (EGFR and MMTV-luc) was compared to progesterone-independent genes (CCNB1 and RBM24) following pre-treatment with example compounds for two hours and six hours of R5020 treatment. The results obtained are shown in FIG. 4e.

It was also shown that NUDT5 inhibitor treatment using example compounds abrogated the progesterone-dependent proliferation response in T47D^{WT} cells as measured by BrdU incorporation. Cells were pre-treated with example compounds for two hours and then stimulated with R5020 for twenty-four hours. These results are shown in FIG. 4f.

### Abbreviations

The following abbreviations may be used herein.

aq aqueous
BINAP 2,2′-bis(diphenylphosphino)-1,1′-binaphthyl
Boc tert-butoxycarbonyl
brine saturated aqueous solution of NaCl
DBU 1,8-diazabicyclo[5.4.0]undec-7-ene
DCM dichloromethane

### DIEA N,N-diisopropylethylamine

DMAP 4-dimethylaminopyridine
DMF dimethylformamide
DMSO dimethylsulfoxide
EDC-HCl N-(3-dimethylaminopropyl)-N′-ethylcarbodiimide hydrochloride
EtOAc ethyl acetate
EtOH ethanol
Ex example
HATU O-(7-azabenzotriazolyl)-N,N,N′,N′-tetramethyluronium hexafluorophosphate
HOBt 1-hydroxybenzotriazole
Int intermediate
LAH lithium aluminium hydride
MeCN acetonitrile
MeOH methanol

### NBS N-bromosuccinimide

NMR nuclear magnetic resonance
Pd₂(dba)₃ tris(dibenzylideneacetone)dipalladium(0)
PBS phosphate buffered saline
rac racemic
rt room temperature
tBuOK potassium tert-butoxide
tBuONa sodium tert-butoxide
TBS tris-buffered saline
TFA trifluoroacetic acid
THF tetrahydrofuran
Xantphos 4,5-bis(diphenylphosphino)-9,9-dimethylxanthene

1. A compound of formula I 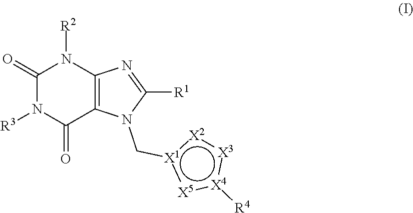
or a pharmaceutically-acceptable salt and/or prodrug thereof, wherein:
any one to three of X¹ to X⁵ represents a heteroatom selected from N, O and S, with the provisos that
only one of X¹ to X⁵ may represent O or S,
if representing a heteroatom, X¹ and X⁴ may only represent N, and
X¹ and X⁴ may not both represent N,
with the remainder of X¹ and X⁴ representing C, and the remainder of X², X³ and X⁵ representing CR⁵;
R¹ represents
(i) heteroaryl optionally substituted by one or more groups selected from E¹, or heterocyclyl optionally substituted by one or more groups independently selected from E², such as wherein each such heteroaryl or heterocyclyl group is attached via a constituent heteroatom,
(ii) —NR^{a1}R^{a2}, —OR^{a3}, —S(O)ₚR^{a4} or —S(O)_{q}NR^{a5},
(iii) C₁₋₁₀ alkyl, C₂₋₁₀ alkenyl or C₂₋₁₀ alkynyl, wherein each such alkyl, alkenyl or alkynyl group is optionally substituted by one or more groups independently selected from E³,
(i) aryl optionally substituted by one or more groups independently selected from E⁴, or
(ii) halo;
R² and R³ each independently represent H, C₁₋₄ alkyl, C₂₋₄ alkenyl or C₂₋₄ alkynyl, wherein each such alkyl, alkenyl or alkynyl group is optionally substituted by one or more groups independently selected from G^{a1};
R⁴ represents aryl optionally substituted by one or more groups independently selected from E⁵, or heteroaryl optionally substituted by one or more groups independently selected from E⁶;
R⁵ represents H, C₁₋₄ alkyl, C₂₋₄ alkenyl or C₂₋₄ alkynyl, wherein each such alkyl, alkenyl or alkynyl group is optionally substituted by one or more groups independently selected from G^{a2}, aryl optionally substituted by one or more groups independently selected from G^{a3}, heterocycyl optionally substituted by one or more groups independently selected from G^{a4}, or heteroaryl optionally substituted by one or more groups independently selected from G^{a5};
R^{a1} to R^{a5} each independently represent C₁₋₁₀ alkyl, C₂₋₁₀ alkenyl or C₂₋₁₀ alkynyl, wherein each such alkyl, alkenyl or alkynyl group is optionally substituted by one or more groups independently selected from G^{b1}, aryl optionally substituted by one or more groups independently selected from G^{b2}, or heteroaryl optionally substituted by one or more groups independently selected from G^{b3}, or
one of R^{a1} and R^{a2} may alternatively represent H;
each of E¹ to E⁶ independently represents halo, ═O, —NR^{b1}R^{b2}, —OR^{b3}, —S(O)ₚR^{b4}, —S(O)_{q}NR^{b5}, —C(O)R^{b6}, —NR^{b7}C(O)R^{b8}, C₁₋₈ alkyl, C₂₋₈ alkenyl or C₂₋₈ alkynyl, wherein each such alkyl, alkenyl or alkynyl group is optionally substituted by one or more groups independently selected from G^{c1}, heterocyclyl optionally substituted by one or more groups independently selected from G^{c2}, or aryl optionally substituted by one or more groups independently selected from G^{c3};
each of G^{a1} to G^{a5}, G^{b1} to G^{b3}, and G^{c1} to G^{c3} independently represent halo, ═O, —NR^{c1}R^{c2}, —OR^{c3}, —S(O)ₚR^{c4}, —S(O)_{q}NR^{c5}, —C(O)R^{c6}, —NR^{c7}C(O)R^{c8}, C₁₋₈ alkyl, C₂₋₈ alkenyl or C₂₋₈ alkynyl, wherein each such alkyl, alkenyl or alkynyl group is optionally substituted by one or more groups independently selected from W^{a1}, heterocyclyl optionally substituted by one or more groups independently selected from W^{a2}, heteroaryl optionally substituted by one or more groups independently selected from W^{a3}, or aryl optionally substituted by one or more groups independently selected from W^{a4};
each of R^{b1} to R^{b8}, and R^{c1} to R^{c8} independently represents H, C₁₋₈ alkyl, C₂₋₈ alkenyl or C₂₋₈ alkynyl, wherein each such alkyl, alkenyl or alkynyl group is optionally substituted by one or more groups independently selected from W^{b1}, heterocyclyl optionally substituted by one or more groups independently selected from W^{b2}, heteroaryl optionally substituted by one or more groups independently selected from W^{b3}, or aryl optionally substituted by one or more groups independently selected from W^{b4},
or alternatively any of R^{b1} and R^{b2}, R^{b7} and R^{b8}, R^{c1} and R^{c2}, and R^{c7} and R^{c8} may be linked, together with the atoms to which they are attached, to form a 4- to 6-membered ring, which ring optionally contains one further heteroatom and which ring optionally is substituted by one or more groups independently selected from halo, C₁₋₃ alkyl optionally substituted by one or more halo, and ═O;
each of W^{a1} to W^{a4}, and W^{b1} to W^{b4} independently represents halo, ═O, —NR^{d1}R^{d2}, —OR^{d3}, —S(O)ₚR^{d4}, —S(O)_{q}NR^{d5}, —C(O)R^{d6}, —NR^{d7}C(O)R^{d8}, C₁₋₈ alkyl, C₂₋₈ alkenyl or C₂₋₈ alkynyl, wherein each such alkyl, alkenyl or alkynyl group is optionally substituted by one or more groups independently selected from Z¹, heterocyclyl optionally substituted by one or more groups independently selected from Z², or aryl optionally substituted by one or more groups independently selected from Z³;
each R^{d1} to R^{d8} independently represents H, C₁₋₃ alkyl, C₂₋₃ alkenyl or C₂₋₃ alkynyl, wherein each such alkyl, alkenyl or alkynyl group is optionally substituted by one or more fluoro;
each Z¹ to Z³ independently represents halo, ═O, —NR^{e1}R^{e2}, —OR^{e3}, —S(O)ₚR^{e4}, —S(O)_{q}NR^{e5}, —C(O)R^{e6}, —NR^{e7}C(O)R^{e8};
each R^{e1} to R^{e8} represents H or C₁₋₃ alkyl, wherein the alkyl group is optionally substituted by one or more fluoro; and
each p and q independently represents 0, 1 or 2.

2. A compound as claimed in claim 1, wherein:
X¹ and X⁴ represent C; and
any one to three of X², X³ and X⁵ represents a heteroatom selected from N, O and S, with the proviso that only one of X², X³ and X⁵ may represent O or S.

3. A compound as claimed in claim 1, wherein:
X¹ and X⁴ represent C;
X² represents N;
X³ represents N or CR⁵; and
X⁵ represents O.

4. A compound as claimed in claim 1,
wherein:
X¹ and X⁴ represent C;
X² represents N;
X³ represents N; and
X⁵ represents O.

5. A compound as claimed in claim 1, wherein R¹ represents:
(i) heteroaryl optionally substituted by one or more groups selected from E¹, or heterocyclyl optionally substituted by one or more groups independently selected from E², such as wherein each such heteroaryl or heterocyclyl group is attached via a constituent heteroatom;
(ii) —NR^{a1}R^{a2}, —OR^{a3}, —S(O)ₚR^{a4} or —S(O)_{q}NR^{a5}; or
(iii) C₁₋₁₀ alkyl, C₂₋₁₀ alkenyl or C₂₋₁₀ alkynyl, wherein each such alkyl, alkenyl or alkynyl group is optionally substituted by one or more groups independently selected from E³.

6. A compound as claimed in claim 1, wherein R¹ represents:
(i) heterocyclyl optionally substituted by one or more groups independently selected from E², such as wherein each such heteroaryl or heterocyclyl group is attached via a constituent heteroatom;
(ii) —NR^{a1}R^{a2}, —OR^{a3}, —S(O)ₚR^{a4} or —S(O)_{q}NR^{a5}, wherein p represents 0 or 2; or
(iii) C₁₋₁₀ alkyl, optionally substituted by one or more groups independently selected from E³.

7. A compound as claimed in claim 1, wherein:
R^{a1} represents H, C₁₋₁₀ alkyl or C₂₋₁₀ alkenyl, each optionally substituted with one or more groups independently selected from G^{b1};
R^{a2} represents C₁₋₁₀ alkyl or C₂₋₁₀ alkenyl, each optionally substituted with one or more groups independently selected from G^{b1};
R^{a3} represents C₁₋₁₀ alkyl, optionally substituted with one or more groups independently selected from G^{b1}; and/or
R^{a4} represents C₁₋₁₀ alkyl, optionally substituted with one or more groups independently selected from G^{b1}.

8. A compound as claimed in claim 1, wherein where R¹ represents heterocyclyl, the heterocyclyl group may:
(a) be saturated; and/or
(b) comprise 4 to 8 atoms.

9. A compound as claimed in claim 1, wherein where R¹ represents heterocyclyl, the heterocyclyl may be selected from:
piperidinyl, octahydro-1H-isoindolyl, azetidinyl, morpholinyl, piperazinyl, azepanyl, pyrrolidinyl, and diazepanyl,
optionally substituted by one or more groups selected from E².

10. A compound as claimed in claim 1, wherein:
(I) each E² group, where present, may represent
halo,
—NR^{b1}R^{b2},
—OR^{b3},
C₁₋₈ alkyl optionally substituted by one or more groups independently selected from G^{c1},
heterocyclyl optionally substituted by one or more groups independently selected from G^{c2}, or
aryl optionally substituted by one or more groups independently selected from G^{c3}; and/or
(II) each E³ group, where present, may represent aryl optionally substituted by one or more groups independently selected from G^{c3}.

11. A compound as claimed in claim 1, wherein:
R^{b1} and R^{b2} may independently represent H or C₁₋₃ alkyl optionally substituted by one or more group selected from ═O and —O^{t}Bu;
R^{b3} may represent H or C₁₋₃ alkyl (e.g. methyl);
G^{c1} may represent ═O, —NR^{c1}R^{c2} or —OR^{c3}, particularly where R^{c1} and R^{c2} represent H and/or (e.g. and) R^{c3} represents H or C₁₋₄ alkyl (e.g. methyl or ^{t}Bu); and/or (e.g. and)
G^{c2} may represent ═O.

12. A compound as claimed in claim 1, wherein R¹ is selected from the following groups: 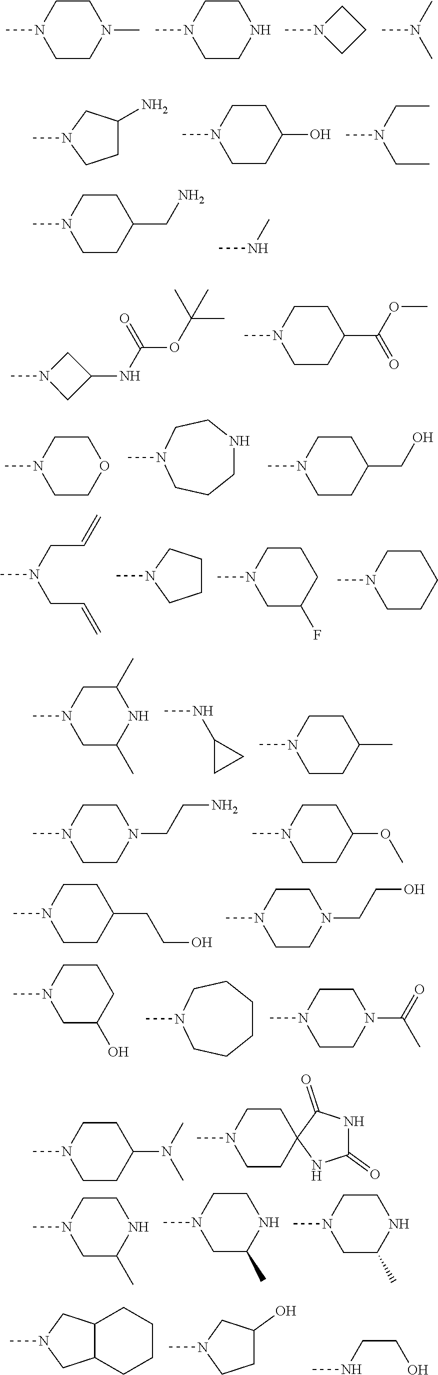 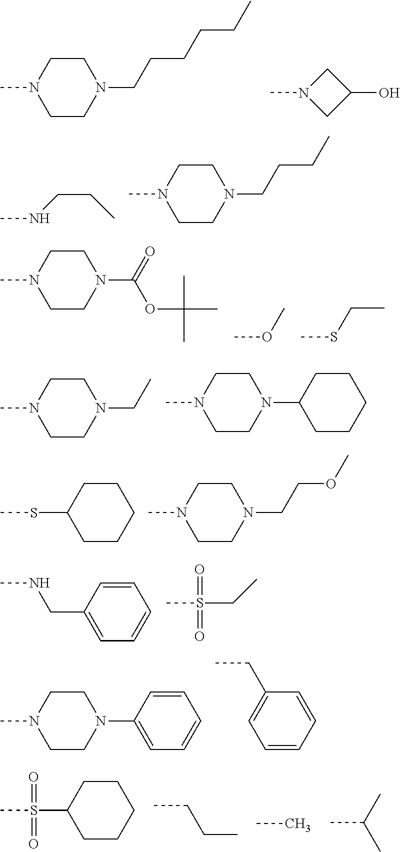
wherein the dashed bond indicates the position of attachment.

13. A compound as claimed in claim 1, wherein:
R² and R³ each independently represent C₁₋₄ alkyl optionally substituted by one or more groups independently selected from G^{a1}.

14. A compound as claimed in claim 1, wherein:
R² and R³ each represent methyl.

15. A compound as claimed in claim 1, wherein:
R⁴ represents aryl optionally substituted by one or more groups independently selected from E⁵.

16. A compound as claimed in claim 1, wherein:
R⁴ represents phenyl optionally substituted by one or more groups independently selected from E⁵.

17. A compound as claimed in claim 1, wherein the compound of formula I is a compound of formula Ia 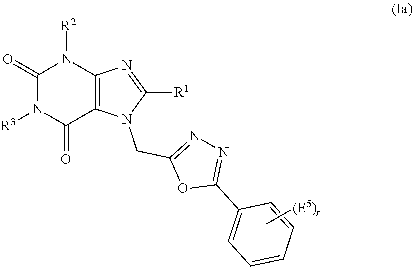
wherein R¹, R², R³ and E⁵ are as defined in claim 1, and wherein r represents 0 to 5.

18. A compound as claimed in claim 1, wherein:
each E⁵ independently represents halo, or C₁₋₈ alkyl optionally substituted by one or more groups independently selected from G^{c1}.

19. A compound as claimed in claim 1, wherein:
each E⁵ independently represents halo, or C₁₋₃ alkyl optionally substituted by one or more groups independently selected from G^{c1}.

20. A compound as claimed in claim 1, wherein:
each E⁵ independently represents chloro or methyl.

21. A compound as claimed in claim 1, wherein the compound of formula I is a compound of formula Ib 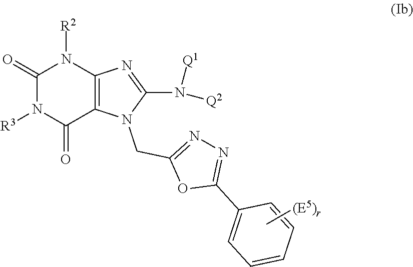
wherein R², R³, E⁵ and r are as defined in claim 1, and wherein:
Q¹ and Q² represent a group as defined herein for R^{a1} and R^{a2}, respectively, or
Q¹ and Q² are linked to form, together with the N to which they are attached, heterocyclyl optionally substituted by one or more groups independently selected from E², as defined herein for R¹.

22-23. (canceled)

24. A method of treating cancer comprising administering to a patient in need thereof a therapeutically effective amount of a compound as defined in claim 1, or a pharmaceutically acceptable salt thereof.

25. (canceled)

26. The method of claim 24, wherein the cancer is a solid tumour cancer, such as a cancer selected from sarcomas, carcinomas, and lymphomas.

27. The method of claim 24, wherein the cancer is a hormone-responsive cancer.

28. The method of claim 24, wherein the cancer is hormone-responsive breast cancer.

29. The method of claim 24, wherein the cancer is oestrogen-responsive (ER) or progesterone-responsive (PR) breast cancer.

30. A pharmaceutical composition comprising a compound as defined in claim 1, or a pharmaceutically acceptable salt thereof, and optionally one or more pharmaceutically-acceptable excipient.

31. (canceled)

32. A combination product comprising:
(I) a compound as defined in claim 1, or a pharmaceutically acceptable salt thereof; and
(II) one or more other therapeutic agent that is useful in the treatment of cancer, wherein each of components (I) and (II) is formulated in admixture, optionally with one or more a pharmaceutically-acceptable excipient.

33. A kit-of-parts comprising:
(a) a pharmaceutical formulation as defined in claim 30; and
(b) one or more other therapeutic agent that is useful in the treatment of cancer, optionally in admixture with one or more pharmaceutically-acceptable excipient, which components (a) and (b) are each provided in a form that is suitable for administration in conjunction with the other.

34. A process for the preparation of a compound as defined in claim 1, or a pharmaceutically acceptable salt thereof, comprising the step of:
(i) reacting a compound of formula II 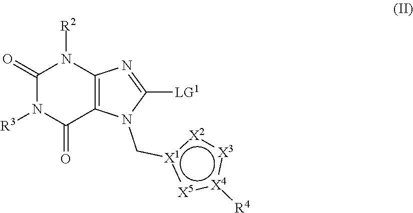
wherein R² to R⁴ and X¹ to X⁵ are as defined in claim 1 and LG¹ represents a suitable leaving group, with a compound of formula III
H—R¹ (III)
wherein R¹ is as defined in claim 1, in the presence of a suitable solvent;
(ii) reacting a compound of formula IV 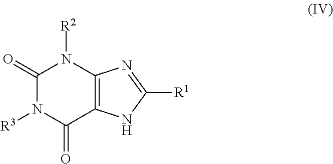
wherein R¹ to R³ are as defined in claim 1, with a compound of formula V 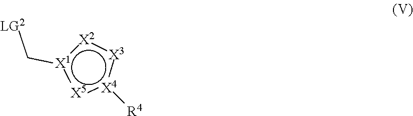
wherein X¹ to X⁵ and R⁴ are as defined in claim 1, and LG² represents a suitable leaving group, in the presence of a suitable solvent and a suitable base;
(iii) reaction of a protected derivative of a compound of formula I as defined in claim 1, or a pharmaceutically acceptable salt thereof, under conditions suitable for the removal of the protecting group(s).

35. A compound as defined in claim 1, selected from
7-{[5-(3,4-dichlorophenyl)-1,3,4-oxadiazol-2-yl]methyl}-8-methoxy-1,3-dimethyl-2,3,6,7-tetrahydro-1H-purine-2,6-dione,
7-{[5-(3,4-dichlorophenyl)-1,3,4-oxadiazol-2-yl]methyl}-8-[(2-hydroxyethyl)amino]-1,3-dimethyl-2,3,6,7-tetrahydro-1H-purine-2,6-dione,
7-{[5-(3,4-dichlorophenyl)-1,3,4-oxadiazol-2-yl]methyl}-8-(dimethylamino)-1,3-dimethyl-2,3,6,7-tetrahydro-1H-purine-2,6-dione,
7-{[5-(3,4-dichlorophenyl)-1,3,4-oxadiazol-2-yl]methyl}-8-(diethylamino)-1,3-dimethyl-2,3,6,7-tetrahydro-1H-purine-2,6-dione,
7-{[5-(3,4-dichlorophenyl)-1,3,4-oxadiazol-2-yl]methyl}-1,3-dimethyl-8-(methylamino)-2,3,6,7-tetrahydro-1H-purine-2,6-dione,
7-{[5-(3,4-dichlorophenyl)-1,3,4-oxadiazol-2-yl]methyl}-8-(ethylsulfanyl)-1,3-dimethyl-2,3,6,7-tetrahydro-1H-purine-2,6-dione,
8-(cyclohexylsulfanyl)-7-{[5-(3,4-dichlorophenyl)-1,3,4-oxadiazol-2-yl]methyl}-1,3-dimethyl-2,3,6,7-tetrahydro-1H-purine-2,6-dione,
8-[bis(prop-2-en-1-yl)amino]-7-{[5-(3,4-dichlorophenyl)-1,3,4-oxadiazol-2-yl]methyl}-1,3-dimethyl-2,3,6,7-tetrahydro-1H-purine-2,6-dione,
7-{[5-(3,4-dichlorophenyl)-1,3,4-oxadiazol-2-yl]methyl}-1,3-dimethyl-8-(piperidin-1-yl)-2,3,6,7-tetrahydro-1H-purine-2,6-dione,
7-{[5-(3,4-dichlorophenyl)-1,3,4-oxadiazol-2-yl]methyl}-1,3-dimethyl-8-(4-methylpiperidin-1-yl)-2,3,6,7-tetrahydro-1H-purine-2,6-dione,
7-{[5-(3,4-dichlorophenyl)-1,3,4-oxadiazol-2-yl]methyl}-1,3-dimethyl-8-(octahydro-1H-isoindol-2-yl)-2,3,6,7-tetrahydro-1H-purine-2,6-dione,
7-{[5-(3,4-dichlorophenyl)-1,3,4-oxadiazol-2-yl]methyl}-1,3-dimethyl-8-{2-oxa-6-azaspiro[3.3]heptan-6-yl}-2,3,6,7-tetrahydro-1H-purine-2,6-dione,
7-{[5-(3,4-dichlorophenyl)-1,3,4-oxadiazol-2-yl]methyl}-1,3-dimethyl-8-(morpholin-4-yl)-2,3,6,7-tetrahydro-1H-purine-2,6-dione,
tert-butyl 4-(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)piperazine-1-carboxylate,
7-{[5-(3,4-dichlorophenyl)-1,3,4-oxadiazol-2-yl]methyl}-8-(4-hexylpiperazin-1-yl)-1,3-dimethyl-2,3,6,7-tetrahydro-1H-purine-2,6-dione,
8-(4-butylpiperazin-1-yl)-7-{[5-(3,4-dichlorophenyl)-1,3,4-oxadiazol-2-yl]methyl}-1,3-dimethyl-2,3,6,7-tetrahydro-1H-purine-2,6-dione,
7-{[5-(3,4-dichlorophenyl)-1,3,4-oxadiazol-2-yl]methyl}-8-(4-ethylpiperazin-1-yl)-1,3-dimethyl-2,3,6,7-tetrahydro-1H-purine-2,6-dione,
7-{[5-(3,4-dichlorophenyl)-1,3,4-oxadiazol-2-yl]methyl}-1,3-dimethyl-8-(4-methylpiperazin-1-yl)-2,3,6,7-tetrahydro-1H-purine-2,6-dione,
7-{[5-(3,4-dichlorophenyl)-1,3,4-oxadiazol-2-yl]methyl}-1,3-dimethyl-8-(piperazin-1-yl)-2,3,6,7-tetrahydro-1H-purine-2,6-dione,
7-{[5-(3,4-dichlorophenyl)-1,3,4-oxadiazol-2-yl]methyl}-8-[4-(2-hydroxyethyl)piperidin-1-yl]-1,3-dimethyl-2,3,6,7-tetrahydro-1H-purine-2,6-dione,
8-(azepan-1-yl)-7-{[5-(3,4-dichlorophenyl)-1,3,4-oxadiazol-2-yl]methyl}-1,3-dimethyl-2,3,6,7-tetrahydro-1H-purine-2,6-dione,
8-(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)-1,3,8-triazaspiro[4.5]decane-2,4-dione,
7-{[5-(3,4-dichlorophenyl)-1,3,4-oxadiazol-2-yl]methyl}-8-(ethanesulfonyl)-1,3-dimethyl-2,3,6,7-tetrahydro-1H-purine-2,6-dione,
8-(cyclohexanesulfonyl)-7-{[5-(3,4-dichlorophenyl)-1,3,4-oxadiazol-2-yl]methyl}-1,3-dimethyl-2,3,6,7-tetrahydro-1H-purine-2,6-dione,
8-(3-aminopyrrolidin-1-yl)-7-{[5-(3,4-dichlorophenyl)-1,3,4-oxadiazol-2-yl]methyl}-1,3-dimethyl-2,3,6,7-tetrahydro-1H-purine-2,6-dione,
tert-butyl N-[1-(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)azetidin-3-yl]carbamate,
8-(1,4-diazepan-1-yl)-7-{[5-(3,4-dichlorophenyl)-1,3,4-oxadiazol-2-yl]methyl}-1,3-dimethyl-2,3,6,7-tetrahydro-1H-purine-2,6-dione,
7-{[5-(3,4-dichlorophenyl)-1,3,4-oxadiazol-2-yl]methyl}-1,3-dimethyl-8-(pyrrolidin-1-yl)-2,3,6,7-tetrahydro-1H-purine-2,6-dione,
7-{[5-(3,4-dichlorophenyl)-1,3,4-oxadiazol-2-yl]methyl}-8-(3,5-dimethylpiperazin-1-yl)-1,3-dimethyl-2,3,6,7-tetrahydro-1H-purine-2,6-dione,
8-[4-(2-aminoethyl)piperazin-1-yl]-7-{[5-(3,4-dichlorophenyl)-1,3,4-oxadiazol-2-yl]methyl}-1,3-dimethyl-2,3,6,7-tetrahydro-1H-purine-2,6-dione,
7-{[5-(3,4-dichlorophenyl)-1,3,4-oxadiazol-2-yl]methyl}-8-[4-(2-hydroxyethyl)piperazin-1-yl]-1,3-dimethyl-2,3,6,7-tetrahydro-1H-purine-2,6-dione,
8-(4-acetylpiperazin-1-yl)-7-{[5-(3,4-dichlorophenyl)-1,3,4-oxadiazol-2-yl]methyl}-1,3-dimethyl-2,3,6,7-tetrahydro-1H-purine-2,6-dione,
7-{[5-(3,4-dichlorophenyl)-1,3,4-oxadiazol-2-yl]methyl}-1,3-dimethyl-8-(3-methylpiperazin-1-yl)-2,3,6,7-tetrahydro-1H-purine-2,6-dione,
7-{[5-(3,4-dichlorophenyl)-1,3,4-oxadiazol-2-yl]methyl}-8-(3-hydroxypyrrolidin-1-yl)-1,3-dimethyl-2,3,6,7-tetrahydro-1H-purine-2,6-dione,
7-{[5-(3,4-dichlorophenyl)-1,3,4-oxadiazol-2-yl]methyl}-8-(3-hydroxyazetidin-1-yl)-1,3-dimethyl-2,3,6,7-tetrahydro-1H-purine-2,6-dione,
8-(azetidin-1-yl)-7-{[5-(3,4-dichlorophenyl)-1,3,4-oxadiazol-2-yl]methyl}-1,3-dimethyl-2,3,6,7-tetrahydro-1H-purine-2,6-dione,
7-{[5-(3,4-dichlorophenyl)-1,3,4-oxadiazol-2-yl]methyl}-8-(4-hydroxypiperidin-1-yl)-1,3-dimethyl-2,3,6,7-tetrahydro-1H-purine-2,6-dione,
8-[4-(aminomethyl)piperidin-1-yl]-7-{[5-(3,4-dichlorophenyl)-1,3,4-oxadiazol-2-yl]methyl}-1,3-dimethyl-2,3,6,7-tetrahydro-1H-purine-2,6-dione,
methyl 1-(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)piperidine-4-carboxylate,
7-{[5-(3,4-dichlorophenyl)-1,3,4-oxadiazol-2-yl]methyl}-8-[4-(hydroxymethyl)piperidin-1-yl]-1,3-dimethyl-2,3,6,7-tetrahydro-1H-purine-2,6-dione,
7-{[5-(3,4-dichlorophenyl)-1,3,4-oxadiazol-2-yl]methyl}-8-(3-fluoropiperidin-1-yl)-1,3-dimethyl-2,3,6,7-tetrahydro-1H-purine-2,6-dione,
8-(cyclopropylamino)-7-{[5-(3,4-dichlorophenyl)-1,3,4-oxadiazol-2-yl]methyl}-1,3-dimethyl-2,3,6,7-tetrahydro-1H-purine-2,6-dione,
7-{[5-(3,4-dichlorophenyl)-1,3,4-oxadiazol-2-yl]methyl}-8-(4-methoxypiperidin-1-yl)-1,3-dimethyl-2,3,6,7-tetrahydro-1H-purine-2,6-dione,
7-{[5-(3,4-dichlorophenyl)-1,3,4-oxadiazol-2-yl]methyl}-8-(3-hydroxypiperidin-1-yl)-1,3-dimethyl-2,3,6,7-tetrahydro-1H-purine-2,6-dione,
7-{[5-(3,4-dichlorophenyl)-1,3,4-oxadiazol-2-yl]methyl}-8-[4-(dimethylamino)piperidin-1-yl]-1,3-dimethyl-2,3,6,7-tetrahydro-1H-purine-2,6-dione,
7-{[5-(3,4-dichlorophenyl)-1,3,4-oxadiazol-2-yl]methyl}-1,3-dimethyl-8-[(3S)-3-methylpiperazin-1-yl]-2,3,6,7-tetrahydro-1H-purine-2,6-dione,
7-{[5-(3,4-dichlorophenyl)-1,3,4-oxadiazol-2-yl]methyl}-1,3-dimethyl-8-[(3R)-3-methylpiperazin-1-yl]-2,3,6,7-tetrahydro-1H-purine-2,6-dione,
8-(dimethylamino)-1,3-dimethyl-7-{[5-(3-methylphenyl)-1,3,4-oxadiazol-2-yl]methyl}-2,3,6,7-tetrahydro-1H-purine-2,6-dione,
1,3-dimethyl-7-{[5-(3-methylphenyl)-1,3,4-oxadiazol-2-yl]methyl}-8-(propylamino)-2,3,6,7-tetrahydro-1H-purine-2,6-dione,
8-(benzylamino)-1,3-dimethyl-7-{[5-(3-methylphenyl)-1,3,4-oxadiazol-2-yl]methyl}-2,3,6,7-tetrahydro-1H-purine-2,6-dione,
1,3-dimethyl-7-{[5-(3-methylphenyl)-1,3,4-oxadiazol-2-yl]methyl}-8-(4-methylpiperazin-1-yl)-2,3,6,7-tetrahydro-1H-purine-2,6-dione,
8-methoxy-1,3-dimethyl-7-{[5-(3-methylphenyl)-1,3,4-oxadiazol-2-yl]methyl}-2,3,6,7-tetrahydro-1H-purine-2,6-dione,
8-(4-hexylpiperazin-1-yl)-1,3-dimethyl-7-{[5-(3-methylphenyl)-1,3,4-oxadiazol-2-yl]methyl}-2,3,6,7-tetrahydro-1H-purine-2,6-dione,
8-(4-butylpiperazin-1-yl)-1,3-dimethyl-7-{[5-(3-methylphenyl)-1,3,4-oxadiazol-2-yl]methyl}-2,3,6,7-tetrahydro-1H-purine-2,6-dione,
8-(4-ethylpiperazin-1-yl)-1,3-dimethyl-7-{[5-(3-methylphenyl)-1,3,4-oxadiazol-2-yl]methyl}-2,3,6,7-tetrahydro-1H-purine-2,6-dione,
8-[4-(2-hydroxyethyl)piperazin-1-yl]-1,3-dimethyl-7-{[5-(3-methylphenyl)-1,3,4-oxadiazol-2-yl]methyl}-2,3,6,7-tetrahydro-1H-purine-2,6-dione,
8-[4-(2-methoxyethyl)piperazin-1-yl]-1,3-dimethyl-7-{[5-(3-methylphenyl)-1,3,4-oxadiazol-2-yl]methyl}-2,3,6,7-tetrahydro-1H-purine-2,6-dione,
1,3-dimethyl-7-{[5-(3-methylphenyl)-1,3,4-oxadiazol-2-yl]methyl}-8-(4-phenylpiperazin-1-yl)-2,3,6,7-tetrahydro-1H-purine-2,6-dione,
8-(4-cyclohexylpiperazin-1-yl)-1,3-dimethyl-7-{[5-(3-methylphenyl)-1,3,4-oxadiazol-2-yl]methyl}-2,3,6,7-tetrahydro-1H-purine-2,6-dione,
7-{[5-(3,4-dichlorophenyl)-1,3,4-oxadiazol-2-yl]methyl}-1,3,8-trimethyl-2,3,6,7-tetrahydro-1H-purine-2,6-dione,
7-{[5-(3,4-dichlorophenyl)-1,3,4-oxadiazol-2-yl]methyl}-1,3-dimethyl-8-propyl-2,3,6,7-tetrahydro-1H-purine-2,6-dione,
7-{[5-(3,4-dichlorophenyl)-1,3,4-oxadiazol-2-yl]methyl}-1,3-dimethyl-8-(propan-2-yl)-2,3,6,7-tetrahydro-1H-purine-2,6-dione,
8-benzyl-7-{[5-(3,4-dichlorophenyl)-1,3,4-oxadiazol-2-yl]methyl}-1,3-dimethyl-2,3,6,7-tetrahydro-1H-purine-2,6-dione,
tert-butyl N-{2-[4-(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)piperazin-1-yl]ethyl}carbamate,
tert-butyl N-{[1-(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)piperidin-4-yl]methyl}carbamate,
7-{[5-(3,4-dichlorophenyl)-1,3,4-oxadiazol-2-yl]methyl}-8-(1H-imidazol-1-yl)-1,3-dimethyl-2,3,6,7-tetrahydro-1H-purine-2,6-dione,
7-{[5-(3,4-dichlorophenyl)-1,3,4-oxadiazol-2-yl]methyl}-1,3-dimethyl-8-(1H-pyrazol-1-yl)-2,3,6,7-tetrahydro-1H-purine-2,6-dione,
ethyl 1-(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)-1H-pyrazole-4-carboxylate,
8-chloro-7-{[3-(4-chlorophenyl)-1,2,4-oxadiazol-5-yl]methyl}-1,3-dimethyl-2,3,6,7-tetrahydro-1H-purine-2,6-dione,
7-{[3-(4-chlorophenyl)-1,2,4-oxadiazol-5-yl]methyl}-1,3-dimethyl-8-(piperazin-1-yl)-2,3,6,7-tetrahydro-1H-purine-2,6-dione,
8-chloro-7-{[2-(3,4-dichlorophenyl)-1,3-oxazol-4-yl]methyl}-1,3-dimethyl-2,3,6,7-tetrahydro-1H-purine-2,6-dione,
7-{[2-(3,4-dichlorophenyl)-1,3-oxazol-4-yl]methyl}-1,3-dimethyl-8-(piperazin-1-yl)-2,3,6,7-tetrahydro-1H-purine-2,6-dione,
8-chloro-7-{[5-(4-methoxyphenyl)-1,3,4-oxadiazol-2-yl]methyl}-1,3-dimethyl-2,3,6,7-tetra hydro-1H-purine-2,6-dione,
7-{[5-(4-methoxyphenyl)-1,3,4-oxadiazol-2-yl]methyl}-1,3-dimethyl-8-(piperazin-1-yl)-2,3,6,7-tetrahydro-1H-purine-2,6-dione,
8-chloro-7-{[2-(4-chlorophenyl)-1,3-thiazol-5-yl]methyl}-1,3-dimethyl-2,3,6,7-tetrahydro-1H-purine-2,6-dione,
7-{[2-(4-chlorophenyl)-1,3-thiazol-5-yl]methyl}-1,3-dimethyl-8-(piperazin-1-yl)-2,3,6,7-tetrahydro-1H-purine-2,6-dione,
8-chloro-7-{[3-(4-methoxyphenyl)-1,2-oxazol-5-yl]methyl}-1,3-dimethyl-2,3,6,7-tetrahydro-1H-purine-2,6-dione,
7-{[3-(4-methoxyphenyl)-1,2-oxazol-5-yl]methyl}-1,3-dimethyl-8-(piperazin-1-yl)-2,3,6,7-tetrahydro-1H-purine-2,6-dione,
1,3-dimethyl-7-{[5-(4-methylphenyl)-1,2-oxazol-3-yl]methyl}-8-(piperazin-1-yl)-2,3,6,7-tetrahydro-1H-purine-2,6-dione,
8-chloro-1,3-dimethyl-7-{[5-(4-methylphenyl)-1,2-oxazol-3-yl]methyl}-2,3,6,7-tetrahydro-1H-purine-2,6-dione,
ethyl 7-(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)-5H,6H,7H,8H-imidazo[1,2-a]pyrazine-2-carboxylate,
2-[(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)amino]ethane-1-sulfonic acid,
7-{[5-(3,4-dichlorophenyl)-1,3,4-oxadiazol-2-yl]methyl}-1,3-dimethyl-8-(3-oxopiperazin-1-yl)-2,3,6,7-tetrahydro-1H-purine-2,6-dione,
tert-butyl 2-[(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)amino]acetate,
4-{[(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)amino]methyl}piperidine-1-carboximidamide,
7-{[5-(3,4-dichlorophenyl)-1,3,4-oxadiazol-2-yl]methyl}-8-{[(2,4-dimethoxyphenyl)methyl]amino}-1,3-dimethyl-2,3,6,7-tetrahydro-1H-purine-2,6-dione,
8-amino-7-{[5-(3,4-dichlorophenyl)-1,3,4-oxadiazol-2-yl]methyl}-1,3-dimethyl-2,3,6,7-tetrahydro-1H-purine-2,6-dione,
8-chloro-7-{[5-(4-chlorophenyl)-1,3,4-oxadiazol-2-yl]methyl}-1,3-dimethyl-2,3,6,7-tetrahydro-1H-purine-2,6-dione,
8-chloro-7-{[5-(4-fluorophenyl)-1,3,4-oxadiazol-2-yl]methyl}-1,3-dimethyl-2,3,6,7-tetrahydro-1H-purine-2,6-dione,
8-chloro-7-{[5-(3-fluorophenyl)-1,3,4-oxadiazol-2-yl]methyl}-1,3-dimethyl-2,3,6,7-tetrahydro-1H-purine-2,6-dione,
8-chloro-7-{[5-(3-methoxyphenyl)-1,3,4-oxadiazol-2-yl]methyl}-1,3-dimethyl-2,3,6,7-tetrahydro-1H-purine-2,6-dione,
4-{5-[(8-chloro-1,3-dimethyl-2,6-dioxo-2,3,6,7-tetrahydro-1H-purin-7-yl)methyl]-1,3,4-oxadiazol-2-yl}benzonitrile
8-chloro-1,3-dimethyl-7-{[5-(pyridin-3-yl)-1,3,4-oxadiazol-2-yl]methyl}-2,3,6,7-tetrahydro-1H-purine-2,6-dione,
8-chloro-7-{[5-(6-chloropyridin-3-yl)-1,3,4-oxadiazol-2-yl]methyl}-1,3-dimethyl-2,3,6,7-tetrahydro-1H-purine-2,6-dione,
8-chloro-1,3-dimethyl-7-{[5-(4-nitrophenyl)-1,3,4-oxadiazol-2-yl]methyl}-2,3,6,7-tetrahydro-1H-purine-2,6-dione,
8-chloro-7-{[5-(4-chloro-3-methylphenyl)-1,3,4-oxadiazol-2-yl]methyl}-1,3-dimethyl-2,3,6,7-tetrahydro-1H-purine-2,6-dione,
8-chloro-7-{[5-(4-chloro-3-methoxyphenyl)-1,3,4-oxadiazol-2-yl]methyl}-1,3-dimethyl-2,3,6,7-tetrahydro-1H-purine-2,6-dione,
8-chloro-7-{[5-(3-chloro-4-methoxyphenyl)-1,3,4-oxadiazol-2-yl]methyl}-1,3-dimethyl-2,3,6,7-tetrahydro-1H-purine-2,6-dione,
tert-butyl 4-(1,3-dimethyl-7-{[5-(4-nitrophenyl)-1,3,4-oxadiazol-2-yl]methyl}-2,6-dioxo-2,3,6,7-tetrahydro-1H-purin-8-yl)piperazine-1-carboxylate,
8-chloro-7-{[5-(1H-indol-6-yl)-1,3,4-oxadiazol-2-yl]methyl}-1,3-dimethyl-2,3,6,7-tetrahydro-1H-purine-2,6-dione,
8-chloro-1,3-dimethyl-7-({5-[4-(trifluoromethyl)phenyl]-1,3,4-oxadiazol-2-yl}methyl)-2,3,6,7-tetrahydro-1H-purine-2,6-dione,
8-chloro-7-({5-[4-(methanesulfonylmethyl)phenyl]-1,3,4-oxadiazol-2-yl}methyl)-1,3-dimethyl-2,3,6,7-tetrahydro-1H-purine-2,6-dione,
8-chloro-7-{[5-(3,4-dimethoxyphenyl)-1,3,4-oxadiazol-2-yl]methyl}-1,3-dimethyl-2,3,6,7-tetrahydro-1H-purine-2,6-dione,
tert-butyl 4-(7-{[5-(4-aminophenyl)-1,3,4-oxadiazol-2-yl]methyl}-1,3-dimethyl-2,6-dioxo-2,3,6,7-tetrahydro-1H-purin-8-yl)piperazine-1-carboxylate,
8-chloro-1,3-dimethyl-7-{[5-(6-methylpyridin-3-yl)-1,3,4-oxadiazol-2-yl]methyl}-2,3,6,7-tetrahydro-1H-purine-2,6-dione,
1,3-dimethyl-7-[(5-phenyl-1,3,4-oxadiazol-2-yl)methyl]-8-(piperazin-1-yl)-2,3,6,7-tetrahydro-1H-purine-2,6-dione,
7-{[5-(4-chlorophenyl)-1,3,4-oxadiazol-2-yl]methyl}-1,3-dimethyl-8-(piperazin-1-yl)-2,3,6,7-tetrahydro-1H-purine-2,6-dione,
7-{[5-(4-fluorophenyl)-1,3,4-oxadiazol-2-yl]methyl}-1,3-dimethyl-8-(piperazin-1-yl)-2,3,6,7-tetrahydro-1H-purine-2,6-dione,
7-{[5-(3-fluorophenyl)-1,3,4-oxadiazol-2-yl]methyl}-1,3-dimethyl-8-(piperazin-1-yl)-2,3,6,7-tetrahydro-1H-purine-2,6-dione,
7-{[5-(3-methoxyphenyl)-1,3,4-oxadiazol-2-yl]methyl}-1,3-dimethyl-8-(piperazin-1-yl)-2,3,6,7-tetrahydro-1H-purine-2,6-dione,
4-(5-{[1,3-dimethyl-2,6-dioxo-8-(piperazin-1-yl)-2,3,6,7-tetrahydro-1H-purin-7-yl]methyl}-1,3,4-oxadiazol-2-yl)benzonitrile
1,3-dimethyl-8-(piperazin-1-yl)-7-{[5-(pyridin-3-yl)-1,3,4-oxadiazol-2-yl]methyl}-2,3,6,7-tetrahydro-1H-purine-2,6-dione,
1,3-dimethyl-8-(piperazin-1-yl)-7-({5-[6-(piperazin-1-yl)pyridin-3-yl]-1,3,4-oxadiazol-2-yl}methyl)-2,3,6,7-tetrahydro-1H-purine-2,6-dione,
1,3-dimethyl-7-{[5-(4-nitrophenyl)-1,3,4-oxadiazol-2-yl]methyl}-8-(piperazin-1-yl)-2,3,6,7-tetrahydro-1H-purine-2,6-dione,
7-{[5-(4-chloro-3-methylphenyl)-1,3,4-oxadiazol-2-yl]methyl}-1,3-dimethyl-8-(piperazin-1-yl)-2,3,6,7-tetrahydro-1H-purine-2,6-dione,
7-{[5-(4-chloro-3-methoxyphenyl)-1,3,4-oxadiazol-2-yl]methyl}-1,3-dimethyl-8-(piperazin-1-yl)-2,3,6,7-tetrahydro-1H-purine-2,6-dione,
7-{[5-(3-chloro-4-methoxyphenyl)-1,3,4-oxadiazol-2-yl]methyl}-1,3-dimethyl-8-(piperazin-1-yl)-2,3,6,7-tetrahydro-1H-purine-2,6-dione,
7-{[5-(3-methoxy-4-methylphenyl)-1,3,4-oxadiazol-2-yl]methyl}-1,3-dimethyl-8-(piperazin-1-yl)-2,3,6,7-tetrahydro-1H-purine-2,6-dione,
7-{[5-(1H-indol-6-yl)-1,3,4-oxadiazol-2-yl]methyl}-1,3-dimethyl-8-(piperazin-1-yl)-2,3,6,7-tetrahydro-1H-purine-2,6-dione,
1,3-dimethyl-8-(piperazin-1-yl)-7-({5-[4-(trifluoromethyl)phenyl]-1,3,4-oxadiazol-2-yl}methyl)-2,3,6,7-tetrahydro-1H-purine-2,6-dione,
7-({5-[4-(methanesulfonylmethyl)phenyl]-1,3,4-oxadiazol-2-yl}methyl)-1,3-dimethyl-8-(piperazin-1-yl)-2,3,6,7-tetrahydro-1H-purine-2,6-dione,
7-{[5-(3,4-dimethoxyphenyl)-1,3,4-oxadiazol-2-yl]methyl}-1,3-dimethyl-8-(piperazin-1-yl)-2,3,6,7-tetrahydro-1H-purine-2,6-dione,
7-{[5-(1H-indazol-5-yl)-1,3,4-oxadiazol-2-yl]methyl}-1,3-dimethyl-8-(piperazin-1-yl)-2,3,6,7-tetrahydro-1H-purine-2,6-dione,
1,3-dimethyl-7-{[5-(6-methylpyridin-3-yl)-1,3,4-oxadiazol-2-yl]methyl}-8-(piperazin-1-yl)-2,3,6,7-tetrahydro-1H-purine-2,6-dione,
8-[4-(dimethylamino)piperidin-1-yl]-7-{[5-(3-methoxy-4-methylphenyl)-1,3,4-oxadiazol-2-yl]methyl}-1,3-dimethyl-2,3,6,7-tetrahydro-1H-purine-2,6-dione,
8-[4-(aminomethyl)piperidin-1-yl]-7-{[5-(3-methoxy-4-methylphenyl)-1,3,4-oxadiazol-2-yl]methyl}-1,3-dimethyl-2,3,6,7-tetrahydro-1H-purine-2,6-dione,
4-{[(7-{[5-(3-methoxy-4-methylphenyl)-1,3,4-oxadiazol-2-yl]methyl}-1,3-dimethyl-2,6-dioxo-2,3,6,7-tetrahydro-1H-purin-8-yl)amino]methyl}piperidine-1-carboximidamide,
7-{[5-(3-methoxy-4-methylphenyl)-1,3,4-oxadiazol-2-yl]methyl}-1,3-dimethyl-8-[3-(trifluoromethyl)piperazin-1-yl]-2,3,6,7-tetrahydro-1H-purine-2,6-dione,
8-[4-(hydroxymethyl)piperidin-1-yl]-7-{[5-(3-methoxy-4-methylphenyl)-1,3,4-oxadiazol-2-yl]methyl}-1,3-dimethyl-2,3,6,7-tetrahydro-1H-purine-2,6-dione,
8-(1,4-diazepan-1-yl)-7-{[5-(3-methoxy-4-methylphenyl)-1,3,4-oxadiazol-2-yl]methyl}-1,3-dimethyl-2,3,6,7-tetrahydro-1H-purine-2,6-dione,
3-[(7-{[5-(3-methoxy-4-methylphenyl)-1,3,4-oxadiazol-2-yl]methyl}-1,3-dimethyl-2,6-dioxo-2,3,6,7-tetrahydro-1H-purin-8-yl)amino]propanamide,
N-{2-[(7-{[5-(3-methoxy-4-methylphenyl)-1,3,4-oxadiazol-2-yl]methyl}-1,3-dimethyl-2,6-dioxo-2,3,6,7-tetrahydro-1H-purin-8-yl)amino]ethyl}methanesulfonamide,
7-{[5-(3,4-dichlorophenyl)-1,3,4-oxadiazol-2-yl]methyl}-1,3-dimethyl-8-[3-(trifluoromethyl)piperazin-1-yl]-2,3,6,7-tetrahydro-1H-purine-2,6-dione,
7-{[5-(3,4-dichlorophenyl)-1,3,4-oxadiazol-2-yl]methyl}-8-{[2-(dimethylamino)ethyl](methyl)amino}-1,3-dimethyl-2,3,6,7-tetrahydro-1H-purine-2,6-dione,
1-(7-{[5-(3,4-dichlorophenyl)-1,3,4-oxadiazol-2-yl]methyl}-1,3-dimethyl-2,6-dioxo-2,3,6,7-tetrahydro-1H-purin-8-yl)piperidine-4-carboxamide,
8-{[2-(dimethylamino)ethyl](methyl)amino}-7-{[5-(3-methoxy-4-methylphenyl)-1,3,4-oxadiazol-2-yl]methyl}-1,3-dimethyl-2,3,6,7-tetrahydro-1H-purine-2,6-dione,
1-(7-{[5-(3-methoxy-4-methylphenyl)-1,3,4-oxadiazol-2-yl]methyl}-1,3-dimethyl-2,6-dioxo-2,3,6,7-tetrahydro-1H-purin-8-yl)piperidine-4-carboxamide,
8-[(3R)-3-(dimethylamino)pyrrolidin-1-yl]-7-{[5-(3-methoxy-4-methylphenyl)-1,3,4-oxadiazol-2-yl]methyl}-1,3-dimethyl-2,3,6,7-tetrahydro-1H-purine-2,6-dione,
tert-butyl N-{2-[(7-{[5-(3-methoxy-4-methylphenyl)-1,3,4-oxadiazol-2-yl]methyl}-1,3-dimethyl-2,6-dioxo-2,3,6,7-tetrahydro-1H-purin-8-yl)sulfanyl]ethyl}carbamate,
N-{2-[(7-{[5-(3-methoxy-4-methylphenyl)-1,3,4-oxadiazol-2-yl]methyl}-1,3-dimethyl-2,6-dioxo-2,3,6,7-tetrahydro-1H-purin-8-yl)sulfanyl]ethyl}acetamide,
8-[(2-aminoethyl)sulfanyl]-7-{[5-(3-methoxy-4-methylphenyl)-1,3,4-oxadiazol-2-yl]methyl}-1,3-dimethyl-2,3,6,7-tetrahydro-1H-purine-2,6-dione,
8-chloro-7-{[1-(3,4-dichlorophenyl)-1H-1,2,3-triazol-4-yl]methyl}-1,3-dimethyl-2,3,6,7-tetrahydro-1H-purine-2,6-dione, and
7-{[1-(3,4-dichlorophenyl)-1H-1,2,3-triazol-4-yl]methyl}-1,3-dimethyl-8-(piperazin-1-yl)-2,3,6,7-tetrahydro-1H-purine-2,6-dione;
or a pharmaceutically acceptable salt thereof.
